SEQUENCE LISTING (49 sequences)

SEQ ID NO: 1 - protein, 116 aa, Artificial Sequence
  Synthetic construct
QVQLVQSGAEVKKPGASVKVSCKASGYYTEAYYIHWVRQAPGQGLEWMGRIDPATGNTKYAPRLQDRVTMTRDTSTSTVYMELSSLRSEDTAVYYCASLYSLPVYWGQGTTVTVSS

SEQ ID NO: 2 - protein, 114 aa, Artificial Sequence
  Synthetic construct
DVVMTQSPLSLPVTLGQPASISCKSSQSLLYSDAKTYLNWFQQRPGQSPRRLIYQISRLDPGVPDRFSGSGSGTDFTLKISRVEAEDVGVYYCLQGTHYPVLFGQGTRLEIKRT

SEQ ID NO: 3 - protein, 10 aa, Artificial Sequence
  Synthetic construct
GYYTEAYYIH

SEQ ID NO: 4 - protein, 17 aa, Artificial Sequence
  Synthetic construct
RIDPATGNTKYAPRLQD

SEQ ID NO: 5 - protein, 7 aa, Artificial Sequence
  Synthetic construct
LYSLPVY

SEQ ID NO: 6 - protein, 16 aa, Artificial Sequence
  Synthetic construct
KSSQSLLYSDAKTYLN

SEQ ID NO: 7 - protein, 7 aa, Artificial Sequence
  Synthetic construct
QISRLDP

SEQ ID NO: 8 - protein, 9 aa, Artificial Sequence
  Synthetic construct
LQGTHYPVL

SEQ ID NO: 9 - DNA, 348 bp, Artificial Sequence
  Synthetic construct
caggtgcagctggtgcagtctggtgctgaggtgaagaagcctggcgcttccgtgaaggtttcctgcaaagcatctggttactatacggaggcttactatatccactgggtgcgccaagcccctggtcaaggcctggagtggatgggcaggattgatcctgcgactggtaatactaaatatgccccgaggttacaggaccgggtgaccatgactcgcgatacctccaccagcactgtctacatggaactgagctctctgcgctctgaggacactgctgtgtattactgtgcctccctttatagtctccctgtctactggggccagggtaccactgttaccgtgtcctct

SEQ ID NO: 10 - DNA, 342 bp, Artificial Sequence
  Synthetic construct
gatgttgtgatgacccagtccccactgtctttgccagttaccctgggacaaccagcctccatatcttgcaagtcaagtcagagcctcttatatagtgatgccaagacatatttgaattggttccaacagaggcctggccagtctccacgccgcctaatctatcagatttcccggctggaccctggcgtgcctgacaggttcagtggcagtggatcaggcacagattttacacttaaaatcagcagagtggaggctgaagatgtgggagtttattactgcttacaaggtacacattatccggtgctcttcggtcaagggacccgcctggagatcaaacgcact

SEQ ID NO: 11 - protein, 442 aa, Artificial Sequence
  Synthetic construct
QVQLVQSGAEVKKPGASVKVSCKASGYYTEAYYIHWVRQAPGQGLEWMGRIDPATGNTKYAPRLQDRVTMTRDTSTSTVYMELSSLRSEDTAVYYCASLYSLPVYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 219 aa, Artificial Sequence
  Synthetic construct
DVVMTQSPLSLPVTLGQPASISCKSSQSLLYSDAKTYLNWFQQRPGQSPRRLIYQISRLDPGVPDRFSGSGSGTDFTLKISRVEAEDVGVYYCLQGTHYPVLFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1336 bp, Artificial Sequence
  Synthetic construct
caggtgcagctggtgcagtctggtgctgaggtgaagaagcctggcgcttccgtgaaggtttcctgcaaagcatctggttactatacggaggcttactatatccactgggtgcgccaagcccctggtcaaggcctggagtggatgggcaggattgatcctgcgactggtaatactaaatatgccccgaggttacaggaccgggtgaccatgactcgcgatacctccaccagcactgtctacatggaactgagctctctgcgctctgaggacactgctgtgtattactgtgcctccctttatagtctccctgtctactggggccagggtaccactgttaccgtgtcctctgcctccaccaagggcccatctgtcttcccactggccccatgctcccgcagcacctccgagagcacagccgccctgggctgcctggtcaaggactacttcccagaacctgtgaccgtgtcctggaactctggcgctctgaccagcggcgtgcacaccttcccagctgtcctgcagtcctcaggtctctactccctcagcagcgtggtgaccgtgccatccagcaacttcggcacccagacctacacctgcaacgtagatcacaagccaagcaacaccaaggtcgacaagaccgtggagagaaagtgttgtgtggagtgtccaccttgtccagcccctccagtggccggaccatccgtgttcctgttccctccaaagccaaaggacaccctgatgatctccagaaccccagaggtgacctgtgtggtggtggacgtgtcccacgaggacccagaggtgcagttcaactggtatgtggacggagtggaggtgcacaacgccaagaccaagccaagagaggagcagttcaactccaccttcagagtggtgagcgtgctgaccgtggtgcaccaggactggctgaacggaaaggagtataagtgtaaggtgtccaacaagggactgccatccagcatcgagaagaccatctccaagaccaagggacagccaagagagccacaggtgtataccctgcccccatccagagaggagatgaccaagaaccaggtgtccctgacctgtctggtgaagggattctatccatccgacatcgccgtggagtgggagtccaacggacagccagagaacaactataagaccacccctccaatgctggactccgacggatccttcttcctgtattccaagctgaccgtggacaagtccagatggcagcagggaaacgtgttctcttgttccgtgatgcacgaggccctgcacaaccactatacccagaagagcctgtccctgtctccaggaaagtaattctaga

SEQ ID NO: 14 - DNA, 666 bp, Artificial Sequence
  Synthetic construct
gatgttgtgatgacccagtccccactgtctttgccagttaccctgggacaaccagcctccatatcttgcaagtcaagtcagagcctcttatatagtgatgccaagacatatttgaattggttccaacagaggcctggccagtctccacgccgcctaatctatcagatttcccggctggaccctggcgtgcctgacaggttcagtggcagtggatcaggcacagattttacacttaaaatcagcagagtggaggctgaagatgtgggagtttattactgcttacaaggtacacattatccggtgctcttcggtcaagggacccgcctggagatcaaacgcactgtggctgcaccatctgtcttcatcttccctccatctgatgagcagttgaaatccggaactgcctctgttgtgtgcctgctgaataacttctatccacgcgaggccaaagtacagtggaaggtggataacgccctccaatccggtaactcccaggagagtgtcacagagcaggacagcaaggacagcacctacagcctcagcagcaccctgaccctgagcaaagcagactacgagaaacacaaagtctacgcctgcgaagtcacccatcagggcctgagttctccagtcacaaagagcttcaaccgcggtgagtgctaattctag

SEQ ID NO: 15 - protein, 40 aa, Homo sapiens
DAEFRHDSGYEVHHQKLVFFAEDVGSNKGAIIGLMVGGVV

SEQ ID NO: 16 - protein, 42 aa, Homo sapiens [sequence omitted]

SEQ ID NO: 17 - protein, 43 aa, Homo sapiens
DAEFRHDSGYEVHHQKLVFFAEDVGSNKGAIIGLMVGGVVIAT

SEQ ID NO: 18 - protein, 41 aa, Homo sapiens
DAEFRHDSGYEVHHQKLVFFAEDVGSNKGAIIGLMVGGVVI

SEQ ID NO: 19 - protein, 39 aa, Homo sapiens
DAEFRHDSGYEVHHQKLVFFAEDVGSNKGAIIGLMVGGV

SEQ ID NO: 20 - protein, 40 aa, Artificial Sequence
  Synthetic construct
DAEFRHDSGYEVHHQKLVFFAEDVGSNKGAIIGLAVGGVV

SEQ ID NO: 21 - protein, 40 aa, Artificial Sequence
  Synthetic construct
DAEFRHDSGYEVHHQKLVFFAEDVGSNKGAIIGLMAGGVV

SEQ ID NO: 22 - protein, 40 aa, Artificial Sequence
  Synthetic construct
DAEFRHDSGYEVHHQKLVFFAEDVGSNKGAIIGLMVAGVV

SEQ ID NO: 23 - protein, 40 aa, Artificial Sequence
  Synthetic construct
DAEFRHDSGYEVHHQKLVFFAEDVGSNKGAIIGLMVGAVV

SEQ ID NO: 24 - protein, 40 aa, Artificial Sequence
  Synthetic construct
DAEFRHDSGYEVHHQKLVFFAEDVGSNKGAIIGLMVGGAV

SEQ ID NO: 25 - protein, 40 aa, Artificial Sequence
  Synthetic construct
DAEFRHDSGYEVHHQKLVFFAEDVGSNKGAIIGLMVGGVA

SEQ ID NO: 26 - protein, 120 aa, Artificial Sequence
  Synthetic construct
QVQLVQSGAEVKKPGASVKVSCKASGYTFTTYAIHWVRQAPGQGLEWMGFTSPYSGVSNYNQKFKGRVTMTRDTSTSTVYMELSSLRSEDTAVYYCARFDNYDRGYVRDYWGQGTLVTVS

SEQ ID NO: 27 - protein, 114 aa, Artificial Sequence
  Synthetic construct
DIVMTQSPDSLAVSLGERATINCRASESVDNDRISFLNWYQQKPGQPPKLLIYAATKQGTGVPDRFSGSGSGTDFTLTISSLQAEDVAVYYCQQSKEFPWSFGGGTKVEIKRTV

SEQ ID NO: 28 - protein, 10 aa, Homo sapiens
GYTFTTYAIH

SEQ ID NO: 29 - protein, 17 aa, Artificial Sequence
  Synthetic construct
FTSPYSGVSNYNQKFKG

SEQ ID NO: 30 - protein, 12 aa, Artificial Sequence
  Synthetic construct
FDNYDRGYVRDY

SEQ ID NO: 31 - protein, 15 aa, Artificial Sequence
  Synthetic construct
RASESVDNDRISFLN

SEQ ID NO: 32 - protein, 7 aa, Artificial Sequence
  Synthetic construct
AATKQGT

SEQ ID NO: 33 - protein, 9 aa, Artificial Sequence
  Synthetic construct
QQSKEFPWS

SEQ ID NO: 34 - DNA, 360 bp, Artificial Sequence
  Synthetic construct
caggtgcaactggtgcaatccggtgccgaggtgaaaaagccaggcgcctccgtgaaagtgtcctgcaaagcctccggttacacctttaccacctatgccatccattgggtgcgccaggccccaggccagggtctggagtggatgggctttacttccccctactccggggtgtcgaattacaatcagaagttcaaaggccgcgtcaccatgacccgcgacacctccacctccacagtgtatatggagctgtcctctctgcgctccgaagacaccgccgtgtattactgtgcccgcttcgacaattacgatcgcggctatgtgcgtgactattggggccagggcaccctggtcaccgtctcc

SEQ ID NO: 35 - DNA, 342 bp, Artificial Sequence
  Synthetic construct
gacatcgtgatgacccagtccccagactccctggccgtgtccctgggcgagcgcgccaccatcaactgccgcgccagcgaatccgtggataacgatcgtatttcctttctgaactggtaccagcagaaaccaggccagcctcctaagctgctcatttacgccgccaccaaacagggtaccggcgtgcctgaccgcttctccggcagcggttccggcaccgatttcactctgaccatctcctccctgcaggccgaagatgtggcagtgtattactgtcagcagtccaaagagtttccctggtcctttggcggtggcaccaaggtggagatcaaacgcactgtg

SEQ ID NO: 36 - protein, 447 aa, Artificial Sequence
  Synthetic construct
QVQLVQSGAEVKKPGASVKVSCKASGYTFTTYAIHWVRQAPGQGLEWMGFTSPYSGVSNYNQKFKGRVTMTRDTSTSTVYMELSSLRSEDTAVYYCARFDNYDRGYVRDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 37 - protein, 218 aa, Artificial Sequence
  Synthetic construct
DIVMTQSPDSLAVSLGERATINCRASESVDNDRISFLNWYQQKPGQPPKLLIYAATKQGTGVPDRFSGSGSGTDFTLTISSLQAEDVAVYYCQQSKEFPWSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 1341 bp, Artificial Sequence
  Synthetic construct
caggtgcaactggtgcaatccggtgccgaggtgaaaaagccaggcgcctccgtgaaagtgtcctgcaaagcctccggttacacctttaccacctatgccatccattgggtgcgccaggccccaggccagggtctggagtggatgggctttacttccccctactccggggtgtcgaattacaatcagaagttcaaaggccgcgtcaccatgacccgcgacacctccacctccacagtgtatatggagctgtcctctctgcgctccgaagacaccgccgtgtattactgtgcccgcttcgacaattacgatcgcggctatgtgcgtgactattggggccagggcaccctggtcaccgtctcctcagcctccaccaagggcccatctgtcttcccactggccccatgctcccgcagcacctccgagagcacagccgccctgggctgcctggtcaaggactacttcccagaacctgtgaccgtgtcctggaactctggcgctctgaccagcggcgtgcacaccttcccagctgtcctgcagtcctcaggtctctactccctcagcagcgtggtgaccgtgccatccagcaacttcggcacccagacctacacctgcaacgtagatcacaagccaagcaacaccaaggtcgacaagaccgtggagagaaagtgttgtgtggagtgtccaccttgtccagcccctccagtggccggaccatccgtgttcctgttccctccaaagccaaaggacaccctgatgatctccagaaccccagaggtgacctgtgtggtggtggacgtgtcccacgaggacccagaggtgcagttcaactggtatgtggacggagtggaggtgcacaacgccaagaccaagccaagagaggagcagttcaactccaccttcagagtggtgagcgtgctgaccgtggtgcaccaggactggctgaacggaaaggagtataagtgtaaggtgtccaacaagggactgccatccagcatcgagaagaccatctccaagaccaagggacagccaagagagccacaggtgtataccctgcccccatccagagaggagatgaccaagaaccaggtgtccctgacctgtctggtgaagggattctatccatccgacatcgccgtggagtgggagtccaacggacagccagagaacaactataagaccacccctccaatgctggactccgacggatccttcttcctgtattccaagctgaccgtggacaagtccagatggcagcagggaaacgtgttctcttgttccgtgatgcacgaggccctgcacaaccactatacccagaagagcctgtccctgtctccaggaaag

SEQ ID NO: 39 - DNA, 654 bp, Artificial Sequence
  Synthetic construct
gacatcgtgatgacccagtccccagactccctggccgtgtccctgggcgagcgcgccaccatcaactgccgcgccagcgaatccgtggataacgatcgtatttcctttctgaactggtaccagcagaaaccaggccagcctcctaagctgctcatttacgccgccaccaaacagggtaccggcgtgcctgaccgcttctccggcagcggttccggcaccgatttcactctgaccatctcctccctgcaggccgaagatgtggcagtgtattactgtcagcagtccaaagagtttccctggtcctttggcggtggcaccaaggtggagatcaaacgcactgtggctgcaccatctgtcttcatcttccctccatctgatgagcagttgaaatccggaactgcctctgttgtgtgcctgctgaataacttctatccacgcgaggccaaagtacagtggaaggtggataacgccctccaatccggtaactcccaggagagtgtcacagagcaggacagcaaggacagcacctacagcctcagcagcaccctgaccctgagcaaagcagactacgagaaacacaaagtctacgcctgcgaagtcacccatcagggcctgagttctccagtcacaaagagcttcaaccgcggtgagtgc

SEQ ID NO: 40 - protein, 118 aa, Artificial Sequence
  Synthetic construct
EVKLVESGGDLVKPGGSLKLSCAASGFTFSTYAMSWIRQTPEKRLEWVASIGNSSRTYYPDSVKGRFTISRDNAGSILYLQMSSLRSEDTAIYYCARGEDGNYAWFTYWGQGTQVTVS

SEQ ID NO: 41 - protein, 106 aa, Artificial Sequence
  Synthetic construct
DIVLTQSPATLSVTPGDSVSLSCRASQSVKNNLHWYQQKSHESPRLLIKYTFQSMSGIPSRFSGSGSGTDFTLIINSVETEDFGMYFCQQSNRWPLTFGAGTKLEL

SEQ ID NO: 42 - protein, 5 aa, Artificial Sequence
  Synthetic construct
TYAMS

SEQ ID NO: 43 - protein, 16 aa, Artificial Sequence
  Synthetic Construct
SIGNSSRTYYPDSVKG

SEQ ID NO: 44 - protein, 11 aa, Artificial Sequence
  Synthetic construct
GEDGNYAWFTY

SEQ ID NO: 45 - protein, 11 aa, Artificial Sequence
  Synthetic construct
RASQSVKNNLH

SEQ ID NO: 46 - protein, 7 aa, Artificial Sequence
  Synthetic construct
YTFQSMS

SEQ ID NO: 47 - protein, 9 aa, Artificial Sequence
  Synthetic construct
QQSNRWPLT

SEQ ID NO: 48 - DNA, 354 bp, Artificial Sequence
  Synthetic construct
gaagtgaagctggtggagtctgggggagacttagtgaagcctggagggtccctgaaactctcctgtgcagcctctggattcactttcagtacctatgccatgtcttggattcgccagactccagagaagaggctggagtgggtcgcctccattggtaatagtagtaggacttactatccagacagtgtgaagggccgattcaccatctccagagataatgccgggagcatcctgtacctccaaatgagcagtctgaggtctgaggacacggccatttattattgtgcaagaggggaagatggtaactacgcctggtttacttactggggccaagggactcaggtcaccgtctcc

SEQ ID NO: 49 - DNA, 318 bp, Artificial Sequence
  Synthetic construct
gatattgtgctaactcagtctccagccaccctgtctgtgactccaggagatagcgtcagtctttcctgcagggccagccaaagtgttaagaacaacctacactggtatcaacaaaagtcacatgagtctccaaggcttctcatcaagtatactttccagtccatgtctgggatcccctccaggttcagtggcagtggctcagggacagatttcactctcattatcaacagtgtggagactgaagattttggaatgtatttctgtcaacagagtaaccgttggccgctcacgttcggtgctgggaccaagctggagctg

(19) United States
(12) Patent Application Publication
(10) Pub. No.: US 2009/0022728 A1
(43) Pub. Date: Jan. 22, 2009

(54) METHODS OF TREATING OPHTHALMIC DISEASES

(75) Inventor: Chia-Yang Lin, Palo Alto, CA (US)

Correspondence Address: PFIZER INC, 10555 SCIENCE CENTER DRIVE, SAN DIEGO, CA, 92121, US

(73) Assignee: RINAT NEUROSCIENCE CORPORATION

(21) Appl. No.: 12/041,581

(22) Filed: Mar. 3, 2008

Related U.S. Application Data
(60) Provisional application No. 60/894,181, filed on Mar. 9, 2007.

(51) Int. Cl. A61K 39/395; A61P 27/02

(52) U.S. Cl. 424/141.1; 424/130.1

(57) ABSTRACT

Methods of using inhibitors (including monoclonal antibodies) directed against amyloid-beta peptide for the treatment of ophthalmic diseases such as age-related macular degeneration are described.

This application claims the benefit of U.S. Provisional Application No. 60/894,181 filed on Mar. 9, 2007, the contents of which is hereby incorporated by reference in its entirety.

### FIELD OF THE INVENTION

The invention concerns methods of using antibodies to amyloid-beta peptide in the treatment and/or prevention of ophthalmic diseases, such as age-related macular degeneration, but also in other ocular pathologies such as glaucoma, diabetic retinopathy (including diabetic macular edema), choroidal neovascular membrane (CNV), uveitis, myopic degeneration, ocular tumors, central retinal vein occlusion, rubeosis, ocular neovascularization, central serous retinopathy, ocular surface discus such as dry eye, central retinal artery occlusion, cystoid macular edema and any other retinal degenerative disease.

### BACKGROUND OF THE INVENTION

The most common cause of decreased best-corrected vision in individuals over 65 years of age in the US is the retinal disorder known as age-related macular degeneration (AMD). As AMD progresses, the disease is characterized by loss of sharp, central vision. The area of the eye affected by AMD is the Macula—a small area in the center of the retina, composed primarily of photoreceptor cells. So-called “dry” AMD (also called “geographic atrophy”), accounting for about 85%-90% of AMD patients, involves alterations in eye pigment distribution, loss of photoreceptors and diminished retinal function due to overall atrophy of cells. So-called “wet” AMD involves proliferation of abnormal choroidal vessels leading to clots or scars in the sub-retinal space. Thus, the onset of wet AMD occurs because of the formation of an abnormal choroidal neovascular network (choroidal neovascularization, CNV) beneath the neural retina. The newly formed blood vessels are excessively leaky. This leads to accumulation of subretinal fluid and blood leading to loss of visual acuity. Eventually, there is total loss of functional retina in the involved region, as a large disciform scar involving choroids and retina forms. While dry AMD patients may retain vision of decreased quality, wet AMD often results in blindness. (Hamdi & Kenney, Age-related Macular degeneration—a new viewpoint, Frontiers in Bioscience, e305-314, May 2003). CNV occurs not only in wet AMD but also in other ocular pathologies such as glaucoma, diabetic retinopathy (including diabetic macular edema), ruptures in Bruch's membrane, myopic degeneration, ocular tumors and other related retinal degenerative diseases.

AMD is a common disorder for which the pathogenesis is clearly multifactorial with genetic and environmental factors playing roles in its onset and progression. Various studies conducted have determined several risk factors for AMD, such as smoking, aging, family history (Milton, Am J Opthalmol 88, 269 (1979); Mitchell et al., Opthalmology 102, 1450-1460 (1995); Smith et al., Opthalmology 108, 697-704 (2001)) sex (7-fold higher likelihood in females: Klein et al., Opthalmology 99, 933-943 (1992) and race (whites are most susceptible). Additional risk factors may include eye characteristics such as farsightedness (hyperopia) and light-colored eyes, as well as cardiovascular disease and hypertension. Evidence of genetic involvement in the onset progression of the disease has also been documented (see Hamdi & Kenney above). Currently, there are no generally accepted animal models for studying AMD. Initial studies by Malek et al. (PNAS 102, 11900-5 (2005)) have produced an animal model having three risk factors that as combined approximated the morphological features of human AMD. Significantly, the development of this mouse model has provided the opportunity to test novel molecular mechanisms and therapeutic targets for AMD. There remains a need to identify novel targets and therapeutic agents capable of treating and/or preventing of ophthalmic diseases such as age-related macular degeneration (both wet and dry), glaucoma, diabetic retinopathy (including diabetic macular edema), choroidal neovascular membrane (CNV), uveitis, myopic degeneration, ocular tumors, entral retinal vein occlusion, rubeosis, ocular neovascularization, central serous retinopathy, ocular surface discus such as dry eye, central retinal artery occlusion, cystoid macular edema and other retinal degenerative disease.

### BRIEF SUMMARY OF THE INVENTION

The present invention discloses novel therapeutic targets implicated in the pathogenesis of ophthalmic diseases. In particular, the present invention discloses methods of treating ophthalmic disease comprising administering to the subject an effective amount of an inhibitor β-amyloid (Aβ) peptide. The Aβ inhibitor may be administered in subjects suffering from ophthalmic diseases such as age-related macular degeneration (both wet and dry ‘AMD’), glaucoma, diabetic retinopathy (including diabetic macular edema), choroidal neovascular membrane (CNV), uveitis, myopic degeneration, ocular tumors, entral retinal vein occlusion, rubeosis, ocular neovascularization, central serous retinopathy, ocular surface discus such as dry eye, central retinal artery occlusion, cystoid macular edema and other retinal degenerative disease. In one embodiment, the inhibitor is an antibody, an antisense molecule, an siRNA molecule, a ribozyme, or a small molecule compound.

In one embodiment, the present invention provides a method of treating a subject suffering from age-related macular degeneration, comprising administering to the subject a pharmaceutical composition comprising a therapeutically effective amount of an inhibitor of β-amyloid (Aβ) peptide. Another embodiment of the present invention concerns a method of treating a subject suffering from age-related macular degeneration (AMD), comprising administering to the subject a pharmaceutical composition comprising a therapeutically effective amount of an Aβ inhibitor.

An additional embodiment of the present invention provides for the use of a therapeutically effective amount of an Aβ inhibitor for the preparation of a medicament for promoting recovery in a patient suffering from AMD. In one aspect of this embodiment, the antibody comprises an Fc region having impaired effector function. In a further aspect of this embodiment, the disease is AMD, including both wet and dry AMD.

The invention also provides methods of treating or preventing diseases associated with amyloid deposit of Aβ, comprising administering to the subject an effective dosage of a pharmaceutical composition comprising an antibody that specifically binds to an Aβ peptide or an aggregated form of an Aβ peptide. In a further aspect of this embodiment, the antibody comprises an Fc region with a variation from a naturally occurring Fc region, wherein the variation results in impaired effector function. In some embodiments, the administration of the antibody causes less cerebral microhemorrhage than administration of an antibody without the variation.

The antibody and polypeptide used for the methods of the invention specifically bind to an Aβ peptide or an aggregated form of an Aβ peptide. In one embodiment, the antibody or polypeptide has impaired effector function. In some embodiments, the antibody or polypeptide is not a F(ab′)₂ fragment. In some embodiments, the antibody or polypeptide is not a Fab fragment. In some embodiments, the antibody or polypeptide is not a single chain antibody scFv.

Polypeptides that specifically bind to an Aβ peptide or an aggregated form of an Aβ peptide and comprises a heavy chain constant region having impaired effector function may also be used for any of the methods described herein. In some embodiments, the polypeptide comprises a sequence (e.g., one or more CDRs) derived from antibody 9TL, 6G or their variants shown in Table 3 or Table 8.

In some embodiments, the antibody or the polypeptide comprises a heavy chain constant region having impaired effector function, wherein the heavy chain constant region comprises an Fc region. In some embodiments, the N-glycosylation in the Fc region is removed. In some embodiments, the Fc region comprises a mutation within the N-glycosylation recognition sequence, whereby the Fc region of the antibody or polypeptide is not N-glycosylated. In some embodiments, the Fc region is PEGylated. In some embodiments, the heavy chain constant region of the antibody or the polypeptide is a human heavy chain IgG2a constant region containing the following mutations: A330P331 to S330S331 (amino acid numbering with reference to the wildtype IgG2a sequence). In some embodiments, the antibody or the polypeptide comprises a constant region of IgG4 comprising the following mutations: E233F234L235 to P233V234A235.

In some embodiments, the antibody or polypeptide specifically binds to an epitope within residues 1-16 of Aβ peptide. In some embodiments, the antibody or polypeptide specifically binds to the N-terminus of the Aβ peptide. In some embodiments, the antibody or the polypeptide specifically binds to an epitope within residues 16-28 of Aβ peptide. In some embodiments, the antibody specifically binds to an epitope on the C-terminal side of an Aβ peptide, such as an epitope starting from amino acid 25 or later. The antibody may specifically bind to any of the Aβ peptides 1-37, 1-38, 1-39, 1-40, 1-41, 1-42, 1-43. In some embodiments, the antibody may specifically bind to the free C-terminus amino acid of C-terminus truncated Aβ peptide, for example, Aβ 1-37, 1-38, 1-39, 1-40, 1-41, 1-42, 1-43. In one embodiment, the antibody or the polypeptide specifically binds to an epitope on the Aβ₁₋₄₀ peptide. In a further aspect of this embodiment, the antibody or the polypeptide specifically binds to an epitope on the Aβ₁₋₄₂ peptide. In a still further aspect of this embodiment, the antibody or the polypeptide specifically binds to an epitope on the Aβ₁₋₄₃ peptide. In some embodiments, the antibody or the polypeptide specifically binds to an epitope within residues 28-40 of Aβ₁₋₄₀ peptide. In some embodiments, the antibody or the polypeptide specifically binds to an epitope within residues 28-42 of Aβ₁₋₄₂ peptide. In some embodiments, the antibody or the polypeptide specifically binds to an epitope within residues 28-43 of Aβ₁₋₄₃ peptide. In some embodiments, the antibody or the polypeptide specifically binds to Aβ peptide without binding to full-length amyloid precursor protein (APP). In some embodiments, the antibody or the polypeptide specifically binds to the aggregated form of Aβ without binding to the soluble form. In some embodiments, the antibody or the polypeptide specifically binds to the soluble form of Aβ without binding to the aggregated form. In some embodiments, the antibody or the polypeptide specifically binds to both aggregated form and soluble forms of Aβ.

In some embodiments, the antibody or the polypeptide specifically binds to a C-terminal peptide 33-40 of Aβ₁₋₄₀. In some embodiments, the antibody or the polypeptide specifically binds to an epitope on Aβ₁₋₄₀ that includes amino acid 35-40. In some embodiments, the antibody or the polypeptide specifically binds to an epitope on Aβ₁₋₄₀ that includes amino acid 36-40. In some embodiments, the antibody or the polypeptide specifically binds to an epitope on Aβ₁₋₄₀ that includes amino acid 39 and/or 40. In some embodiments, the antibody or the polypeptide specifically binds to Aβ₁₋₄₀ but do not specifically bind to Aβ₁₋₄₂ and/or Aβ₁₋₄₃. In some embodiments, the antibody comprises the variable region of antibody 9TL or an antibody derived from 9TL described herein. In some embodiments, the antibody or polypeptide competitively inhibits binding of antibody 9TL, 6G and/or antibody or polypeptide derived from 9TL or 6G to the respective Aβ peptide.

In some embodiments, the antibody or the polypeptide binds to Aβ₁₋₄₀ with higher affinity than its binding to Aβ₁₋₄₂ and Aβ₁₋₄₃. In a further aspect of this embodiment, the antibody is not antibody 2294. In some embodiments, the antibody binds to an epitope on Aβ₁₋₄₀ that includes amino acids 25-34 and 40. In some embodiments, the antibody comprises the variable region of antibody 6G or an antibody derived from 6G described herein. In some embodiments, the antibody or polypeptide competitively inhibits binding of antibody 6G and/or antibody or polypeptide derived from 6G to Aβ.

In some embodiments, the antibody or the polypeptide binds to the Aβ peptide with a binding affinity (K_{D}) of about 100 nM or less, or 20 nM or less, or 2 nM or less. In one aspect of this embodiment, the antibody or polypeptide binds to the Aβ₁₋₄₀ peptide with a K_{D} of about 100 nM or less, 50 nM or less, or 2 nM or less. In a further aspect of this embodiment, the antibody or polypeptide also binds to the Aβ₁₋₄₂ peptide with a K_{D} of about 100 nM or less, 50 nM or less, or 2 nM or less.

Administration of antibody or polypeptide that specifically binds to an Aβ peptide may be by any means known in the art, including: intravenously, subcutaneously, via inhalation, intraarterially, intramuscularly, intracardially, intraventricularly, parenteral, intrathecally, and intraperitoneally. Administration may be by injection and/or systemic, e.g. intravenously, or localized. This also generally applies to polypeptides and polynucleotides of the invention.

The invention also provides methods of treating ophthalmic disease by administering pharmaceutical composition comprising an effective amount of any of the antibodies or polypeptides that specifically bind to an Aβ peptide or an aggregated form of an Aβpeptide and have impaired effector function, or polynucleotides encoding the antibodies or polypeptides, and a pharmaceutical acceptable excipient.

The invention also provides kits and compositions comprising any one or more of the compositions comprising an effective amount of any of the antibodies or polypeptides that specifically bind to an Aβ peptide or an aggregated form of an Aβ peptide, or polynucleotides encoding the antibodies or polypeptides. These kits, generally in suitable packaging and provided with appropriate instructions, are useful for any of the methods described herein.

The invention also provides a method of producing a therapeutic humanized antibody for treatment of a disease associated with amyloid deposits of Aβ peptide in the brain of a human subject, comprising selecting a first humanized antibody that specifically binds to Aβ peptide; and altering the Fc region of the antibody to provide a therapeutic humanized antibody having impaired effector function relative to the first humanized antibody.

Another embodiment of the present invention is directed to a method for protecting or recovering retinal function in a subject, comprising administering to the subject a pharmaceutical composition comprising a therapeutically effective amount of an Aβ inhibitor. In one embodiment, the inhibitor is an antibody, an antisense molecule, an siRNA molecule, a ribozyme, or a small molecule compound.

Another embodiment of the present invention is directed to a method for preserving or restoring visual acuity in a subject, comprising a therapeutically effective amount of an Aβ inhibitor.

In on aspect of the above embodiments, the above methods are used in subjects which are not also being treated for Alzheimer's disease, Down's syndrome, or cerebral amyloid angiopathy.

The above-mentioned methods of the invention include an Aβ inhibitor which is an antibody. In one aspect, the invention disclosed herein concerns antibodies that bind to C-terminus of Aβ 1-40 peptide (SEQ ID NO:15 shown in Table 4). Accordingly, in one aspect, the methods comprise treatment with an antibody 9TL (interchangeably termed “9TL”) that is produced by expression vectors having ATCC Accession Nos. PTA-6124 and PTA-6125. The amino acid sequences of the heavy chain and light chain variable regions of 9TL are shown in FIG. 1. The complementarity determining region (CDR) portions of antibody 9TL (including Chothia and Kabat CDRs) are also shown in FIG. 1. It is understood that reference to any part of or entire region of 9TL encompasses sequences produced by the expression vectors having ATCC Accession Nos. PTA-6124 and PTA-6125, and/or the sequences depicted in FIG. 1.

In another aspect, the invention comprises administration of antibody variants of 9TL with amino acid sequences depicted in Table 3.

In another aspect, the invention comprises administration of an antibody comprising a fragment or a region of the antibody 9TL or its variants shown in Table 3. In one embodiment, the fragment is a light chain of the antibody 9TL. In another embodiment, the fragment is a heavy chain of the antibody 9TL. In yet another embodiment, the fragment contains one or more variable regions from a light chain and/or a heavy chain of the antibody 9TL. In yet another embodiment, the fragment contains one or more variable regions from a light chain and/or a heavy chain shown in FIG. 1. In yet another embodiment, the fragment contains one or more CDRs from a light chain and/or a heavy chain of the antibody 9TL.

In another aspect, the invention comprises administration of polypeptides (which may or may not be an antibody) comprising any one or more of the following: a) one or more CDR(s) of antibody 9TL or its variants shown in Table 3; b) CDR H3 from the heavy chain of antibody 9TL or its variants shown in Table 3; c) CDR L3 from the light chain of antibody 9TL or its variants shown in Table 3; d) three CDRs from the light chain of antibody 9TL or its variants shown in Table 3; e) three CDRs from the heavy chain of antibody 9TL or its variants shown in Table 3; f) three CDRs from the light chain and three CDRs from the heavy chain of antibody 9TL or its variants shown in Table 3. The invention further provides administration of polypeptides (which may or may not be an antibody) comprising any one or more of the following: a) one or more (one, two, three, four, five, or six) CDR(s) derived from antibody 9TL or its variants shown in Table 3; b) a CDR derived from CDR H3 from the heavy chain of antibody 9TL; and/or c) a CDR derived from CDR L3 from the light chain of antibody 9TL. In some embodiments, the CDR is a CDR shown in FIG. 1. In some embodiments, the one or more CDRs derived from antibody 9TL or its variants shown in Table 3 are at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least one, at least two, at least three, at least four, at least five, or at least six CDRs of 9TL or its variants.

In another aspect, the invention comprises administration of an antibody 6G (interchangeably termed “6G”). The amino acid sequences of the heavy chain and light chain variable regions of 6G are shown in FIG. 8. The complementarity determining region (CDR) portions of antibody 6G (including Chothia and Kabat CDRs) are also shown in FIG. 8.

In another aspect, the invention comprises administration of antibody variants of 6G with amino acid sequences depicted in Table 8.

In another aspect, the invention comprises administration of an antibody comprising a fragment or a region of the antibody 6G or its variants shown in Table 8. In one embodiment, the fragment is a light chain of the antibody 6G. In another embodiment, the fragment is a heavy chain of the antibody 6G. In yet another embodiment, the fragment contains one or more variable regions from a light chain and/or a heavy chain of the antibody 6G. In yet another embodiment, the fragment contains one or more variable regions from a light chain and/or a heavy chain shown in FIG. 8. In yet another embodiment, the fragment contains one or more CDRs from a light chain and/or a heavy chain of the antibody 6G.

In another aspect, the invention comprises administration of polypeptides (which may or may not be an antibody) comprising any one or more of the following: a) one or more CDR(s) of antibody 6G or its variants shown in Table 8; b) CDR H3 from the heavy chain of antibody 6G or its variants shown in Table 8; c) CDR L3 from the light chain of antibody 6G or its variants shown in Table 8; d) three CDRs from the light chain of antibody 6G or its variants shown in Table 8; e) three CDRs from the heavy chain of antibody 6G or its variants shown in Table 8; f) three CDRs from the light chain and three CDRs from the heavy chain of antibody 6G or its variants shown in Table 8. The invention further comprises administration of polypeptides (which may or may not be an antibody) comprising any one or more of the following: a) one or more (one, two, three, four, five, or six) CDR(s) derived from antibody 6G or its variants shown in Table 8; b) a CDR derived from CDR H3 from the heavy chain of antibody 6G; and/or c) a CDR derived from CDR L3 from the light chain of antibody 6G. In some embodiments, the CDR is a CDR shown in FIG. 8. In some embodiments, the one or more CDRs derived from antibody 6G or its variants shown in Table 8 are at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least one, at least two, at least three, at least four, at least five, or at least six CDRs of 6G or its variants.

In a further aspect, the invention comprises administration of an antibody comprising a heavy chain variable region comprising three CDRs from antibody 6G heavy chain variable region shown in SEQ ID NO:26, and a light chain variable region comprising three CDRs from antibody 6G light variable region shown in SEQ ID NO:27. In another aspect, the invention comprises administration of an antibody comprising a heavy chain variable region comprising the three CDRs shown in SEQ ID NO:28, SEQ ID NO:29, and SEQ ID NO:30, and a light chain variable region comprising the three CDRs shown in SEQ ID NO:31, SEQ ID NO:32, and SEQ ID NO:33. In still another aspect, the invention comprises a heavy chain variable region comprising the amino acid sequence shown in SEQ ID NO:26, and a light chain variable region comprising the amino acid sequence shown in SEQ ID NO:27. In still another aspect, the invention comprises a heavy chain amino acid sequence shown in SEQ ID NO:36, and the light chain amino acid sequence shown in SEQ ID NO:37.

In some embodiments, the CDR is a Kabat CDR. In other embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined.

In some embodiments, the polypeptide (such as an antibody) comprises an amino acid sequence shown in SEQ ID NO:5, wherein L1 is L, V, or I; wherein Y2 is Y or W; wherein S3 is S, T, or G; wherein L4 is L, R, A, V, S, T, Q, or E; wherein V6 is V, I, T, P, C, Q, S, N, or F; and wherein Y7 is Y, H, F, W, S, I, V, or A. In some embodiments, the amino acid sequence is a CDR3 in a heavy chain variable region. For convenience herein, “is” in this context or reference to an amino acid refers to choices of amino acid(s) for a given position with reference to the position in the SEQ ID. For example, “L1 is L, V, or I” refers to amino acid L at position 1 in SEQ ID NO:5 may be substituted with V or 1.

In some embodiments, the polypeptide (such as an antibody) comprises an amino acid sequence shown in SEQ ID NO:6, wherein Y8 is Y, A, or H; and wherein A11 is A or S; and wherein K12 is K or A. In some embodiments, the amino acid sequence is a CDR1 in a light chain variable region.

In some embodiments, the polypeptide (such as an antibody) comprises an amino acid sequence shown in SEQ ID NO:8, wherein L1 is L, M, N, C, F, V, K, S, Q, G, S; wherein G3 is G, S, or T; wherein T4 is T or S; wherein H5 is H or L; wherein Y6 is Y, P, A, W, Q, M, S, or E; wherein V8 is V, L, K, H, T, A, E, or M; and wherein L9 is L, I, T, S, or V. In some embodiments, the amino acid sequence is a CDR3 in a light chain variable region.

In some embodiments, the polypeptide (such as an antibody) comprises a heavy chain variable region comprising (a) a CDR1 region shown in SEQ ID NO:3; (b) a CDR2 region shown in SEQ ID NO:4; and (c) a CDR3 region shown in SEQ ID NO:5, wherein L1 is L, V, or I; wherein Y2 is Y or W; wherein S3 is S, T, or G; wherein L4 is L, R, A, V, S, T, Q, or E; wherein V6 is V, I, T, P, C, Q, S, N, or F; and wherein Y7 is Y, H, F, W, S, I, V, or A.

In some embodiments, the polypeptide (such as an antibody) comprises a light chain variable region comprising (a) a CDR1 region shown in SEQ ID NO:6, wherein Y8 is Y, A, or H; and wherein A11 is A or S; and wherein K12 is K or A; (b) a CDR2 region shown in SEQ ID NO:7; and (c) a CDR3 region shown in SEQ ID NO:8, wherein L1 is L, M, N, C, F, V, K, S, Q, G, S; wherein G3 is G, S, or T; wherein T4 is T or S; wherein H5 is H or L; wherein Y6 is Y, P, A, W, Q, M, S, or E; wherein V8 is V, L, K, H, T, A, E, or M; and wherein L9 is L, I, T, S, or V.

In some embodiments, the antibody of the invention is a human antibody. In other embodiments, the antibody of the invention is a humanized antibody. In some embodiments, the antibody is monoclonal. In some embodiments, the antibody (or polypeptide) is isolated. In some embodiments, the antibody (or polypeptide) is substantially pure.

The heavy chain constant region of the antibodies may be from any types of constant region, such as IgG, IgM, IgD, IgA, and IgE; and any isotypes, such as IgG1, IgG2, IgG3, and IgG4. In some embodiments, the antibody comprises a modified constant region, such as a constant region that is immunologically inert (which includes partially immunologically inert, and is used interchangeably with the term “having impaired effector function”), e.g., does not trigger complement mediated lysis, does not stimulate antibody-dependent cell mediated cytotoxicity (ADCC), or does not activate microglia. In some embodiments, the constant region is modified as described in Eur. J. Immunol. (1999) 29:2613-2624; PCT Application No. PCT/GB99/01441; and/or UK Patent Application No. 9809951.8. In other embodiments, the antibody comprises a human heavy chain IgG2a constant region comprising the following mutations: A330P331 to S330S331 (amino acid numbering with reference to the wildtype IgG2a sequence). Eur. J. Immunol. (1999) 29:2613-2624. In some embodiments, the antibody comprises a constant region of IgG4 comprising the following mutations: E233F234L235 to P233V234A235. In still other embodiments, the constant region is aglycosylated for N-linked glycosylation. In some embodiments, the constant region is aglycosylated for N-linked glycosylation by mutating the oligosaccharide attachment residue (such as Asn297) and/or flanking residues that are part of the N-glycosylation recognition sequence in the constant region. In some embodiments, the constant region is aglycosylated for N-linked glycosylation. The constant region may be aglycosylated for N-linked glycosylation enzymatically or by expression in a glycosylation deficient host cell.

In another aspect, the invention provides a polynucleotide (which may be isolated) comprising a polynucleotide encoding a fragment or a region of the antibody 9TL or 6G or their variants shown in Table 3 and Table 8. In one embodiment, the fragment is a light chain of the antibody 9TL or 6G. In another embodiment, the fragment is a heavy chain of the antibody 9TL or 6G. In yet another embodiment, the fragment contains one or more variable regions from a light chain and/or a heavy chain of the antibody 9TL or 6G. In yet another embodiment, the fragment contains one or more (i.e., one, two, three, four, five, six) complementarity determining regions (CDRs) from a light chain and/or a heavy chain of the antibody 9TL or 6G.

### BRIEF DESCRIPTION OF THE SEVERAL VIEWS OF THE DRAWING(S)

FIG. 1 shows the amino acid sequence of the heavy chain variable region (SEQ ID NO:1) and light chain variable region (SEQ ID NO:2) of the 9TL antibody. The Kabat CDRs are in bold text, and the Chothia CDRs are underlined. The amino acid residues for the heavy chain and light chain variable region are numbered sequentially.

FIG. 2 shows epitope mapping of antibody 9TL by peptide competition. Aβ₁₋₄₀ peptide was immobilized on the SA chip. Monoclonal antibody 2289 and 9TL Fab fragment (50 nM each), each of which was preincubated for 1 h with 10 μM various peptide (amino acids 28-40, 1-40, 1-28, 28-42, 22-35, 1-16, 1-43, 33-40, 1-38, or 17-40 of Aβ) or no peptide, and was then flowed onto the chip. Binding of the antibody Fab fragment to immobilized Aβ₁₋₄₀ peptide was measured.

FIG. 3 is a graph showing epitope mapping of antibody 2H6 by peptide competition. Aβ₁₋₄₀ peptide was immobilized on the SA chip. Monoclonal antibody 2289, 2286, or 2H6 (100 nM each), each of which was preincubated for 1 h with 16 μM various peptide (amino acids 1-16, 1-28, 1-38, 1-40, 1-42, 1-43, 17-40, 17-42, 22-35, 25-35, or 33-40 of Aβ) or no peptide, was flowed onto the chip. Binding of the antibody to immobilized Aβ₁₋₄₀ peptide was measured.

FIG. 4 is a graph showing binding of antibody 2H6, 2286, and 2289 to different Aβ peptide C-terminal variants. GST-Aβ variants (M35A, V36A, G37A, G38A, V39A, or V40A), or GST-Aβ peptide 1-39, 1-41, 1-40, 1-42 were immobilized on ELISA plate. Monoclonal antibody 2286, 2H6, or 2289 (0.3 nM each mAb) was incubated with each of the immobilized peptides, and their binding was detected by further incubating with biotinylated anti-mouse IgG (H+L) and followed by Sterptavidin-HRP.

FIG. 5 is a graph of intensity of a- and b-waves (A) and sample electroretinograms (B) from aged apolipoprotein isoform E4 (APOE4) mice on normal versus high fat and cholesterol diet.

FIG. 6 is a graph of intensity of b-waves only of APOE4 mice plotted against previous studies of normal diet animals. The R2 trace shows the protection or recovery of retinal function when AMD-like mice (E4-HFC-R2) were treated with anti-Aβ antibody.

FIG. 7 shows total Aβ immunohistochemistry of AMD-like (APOE4) mouse brain. Slide A (AMD-like mouse treated with anti-Aβ antibody) shows negative amyloid detection. Slides B, C, and D (AMD-like mouse treated with vehicle injection) shows positive amyloid detection. Slide E is taken from a positive control, and is taken from the brain of a platelet-derived APP mouse model (pdAPP, mutated (V717F) human APP under the control of platelet-derived growth factor promoter (Games, D. et al, Nature 373: 523-527 (1995)).

FIG. 8 shows the amino acid sequence of the heavy chain variable region (SEQ ID NO:1) and light chain variable region (SEQ ID NO:27) of the 6G antibody. The Kabat CDRs are in bold text, and the Chothia CDRs are underlined. The amino acid residues for the heavy chain and light chain variable region are numbered sequentially.

FIG. 9 shows epitope mapping of antibody 6G by ELISA. Aβ peptides (1-16, 1-28, 17-40, 17-42, 22-35, 28-40, 28-42, 1-38, 1-40, 1-42, 1-43, and 33-40) were immobilized on ELISA plates. Monoclonal antibody 6G (20 nM) was incubated for 1 h with various immobilized peptides. Antibody 6G bound to immobilized Aβ peptides was measured using goat anti-human kappa HRP conjugated secondary antibody.

FIG. 10 shows epitope mapping of antibody 6G by ELISA. Various Aβ peptides were immobilized on ELISA plates. Antibody 6G was incubated for 1 h with various immobilized peptides. Antibody 6G bound to immobilized Aβ peptides was measured using goat anti-human kappa HRP conjugated secondary antibody. “NB” refers to no binding detected.

FIG. 11 is a schematic graph showing epitope that antibody 6G binds on Aβ. Relative positions of Aβ in amyloid precursor protein (APP) and portion of APP in cell membrane are shown. “CT99” refers to C-terminal 99 amino acids of APP.

FIG. 12 is a photograph showing immunostaining of APP expression cells with monoclonal antibody directed to Aβ₁₋₁₆ (m2324) and antibody 6G. The top panels show cells under fluorescence microscope after the cells were incubated with m2324 or 6G (each 5 ug/ml) and binding was detected by secondary Cy3-conjugated goat anti-mouse or anti-human antibody. The bottom panels show cells observed under microscope.

FIG. 13 is a graph of intensity of b-waves only of five study groups of APOE4 mice: control APOE4 mice on a normal diet; control APOE4 mice on a high fat and cholesterol diet (‘HFC’)(the AMD-like model); APOE4-HFC mice treated with 7G10; APOE4-HFC mice treated with 2H6; and APOE4-HFC mice treated with 6G.

FIG. 14 is a graph of intensity of b-waves only of three study groups of APOE4 mice: control APOE4 mice on a normal diet; control APOE4 HFC mice; and APOE4-HFC mice treated with 6G.

### DETAILED DESCRIPTION OF THE INVENTION

The mouse model of AMD has been instrumental in testing the hypothesis that, without being bound by theory, lipid transport dysregulation and amyloid deposition may contribute to the pathogenesis of observed retinal changes seen in age-related macular degeneration, glaucoma, diabetic retinopathy (including macular edema) and other related retinal degenerative diseases. Aβ deposition has been extensively studied in Alzheimers, and previous studies have indicated a potential role of Aβ in age-related macular degeneration (Yoshida, T., et al, J. of Clin. Invest., 115(10): 2793-2800 (2005); Anderson, D. et al, Experimental Eye Research 78: 243-256 (2004); Johnson, L. et al, PNAS, 99(18): 11820-11835 (2002)) and glaucoma (McKinnon S J, Front Biosci 8: 1140-56 (2003); Tatton et al, Surv Opthalmol. 48: S25-37 (2003)). However, there has thus far been no discussion as to whether an inhibitor of Aβ may provide therapeutic benefit in the treatment of macular degeneration by effecting retinal protection and/or recovery. Furthermore, there has been no discussion as to whether any of the isoforms of Aβ may differentially contribute to the pathogenesis of AMD.

As discussed above, Aβ is the major constituent of the neuritic plaques found in Alzheimer's disease. Aβ is the cleavage product of beta amyloid precursor protein (βAPP or APP). APP is a type I transmembrane glycoprotein that contains a large ectopic N-terminal domain, a transmembrane domain, and a small cytoplasmic C-terminal tail. Alternative splicing of the transcript of the single APP gene on chromosome 21 results in several isoforms that differ in the number of amino acids. Previous studies in Alzeimer's disease has established that the Aβ₁₋₄₂ isoform is essential for amyloid deposition and that Aβ₁, 42, as opposed to Aβ₁₋₄₀, may be the initiating molecule in the pathogenesis of Alzheimer's (McGowan, E. et al, Neuron 47: 191-199 (2005). Additional studies in Alzheimer's disease furthermore suggest that the Aβ₁₋₄₀ isoform may actually inhibit amyloid deposition, and that an inhibitor of Aβ₁₋₄₀ could worsen Alzheimer's disease course (Kim, J. et al, Neurobiology of Disease, 27(3): 627-633 (2007).

The invention disclosed herein provides methods for preventing and/or treating ophthalmic diseases such as age-related macular degeneration (both wet and dry), glaucoma, diabetic retinopathy (including diabetic macular edema), ruptures in Bruch's membrane, myopic degeneration, ocular tumors and other related retinal degenerative diseases in an individual by administration of a therapeutically effective amount of an antibody 9TL, or 6G, or antibody or polypeptide derived therefrom. The antibody 9TL and its derivatives have been described in WO 2006036291, the disclosure of which is hereby incorporated by reference in its entirety. The antibodies and polypeptides used in the disclosed methods bind to the C-terminus of Aβ₁₋₄₀. The antibody 6G and its derivatives have been described in both WO 2006036291 and WO 2006118959, the disclosures of which are hereby incorporated by reference in their entireties. The methods of the invention are intended to include all inhibitors of Aβ, including but not limited to small molecule compounds, and biologics such as antibodies, antisense molecules, siRNA molecules and ribozymes.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### DEFINITIONS

An “inhibitor of Aβ peptide” is any agent capable of decreasing Aβ peptide production and/or deposition. An inhibitor of Aβ peptide includes, but is not limited to, an antibody, an antisense molecule, an siRNA molecule, a ribozyme, or a small molecule compound. Moreover, an inhibitor of Aβ peptide is any agent capable of binding Aβ peptide and decreasing Aβ plaque deposition, including any agents capable of disrupting proteolytic cleavage of amyloid precursor protein into the product Aβ peptides. Additional targets for inhibition of Aβ peptide production and deposition include, but are not limited to, for example, small molecule therapeutics or siRNA capable of inhibiting or silencing β-secretase (also called BACE1 or memapsin-2) or the gamma secretase complex (which minimally consists of four individual proteins: presenilin, nicastrin, anterior pharynx-defective 1 (APH-1) and presenilin enhancer 2 (PEN-2).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

As used herein, “humanized” antibodies refer to forms of non-human (e.g. murine) antibodies that are specific chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

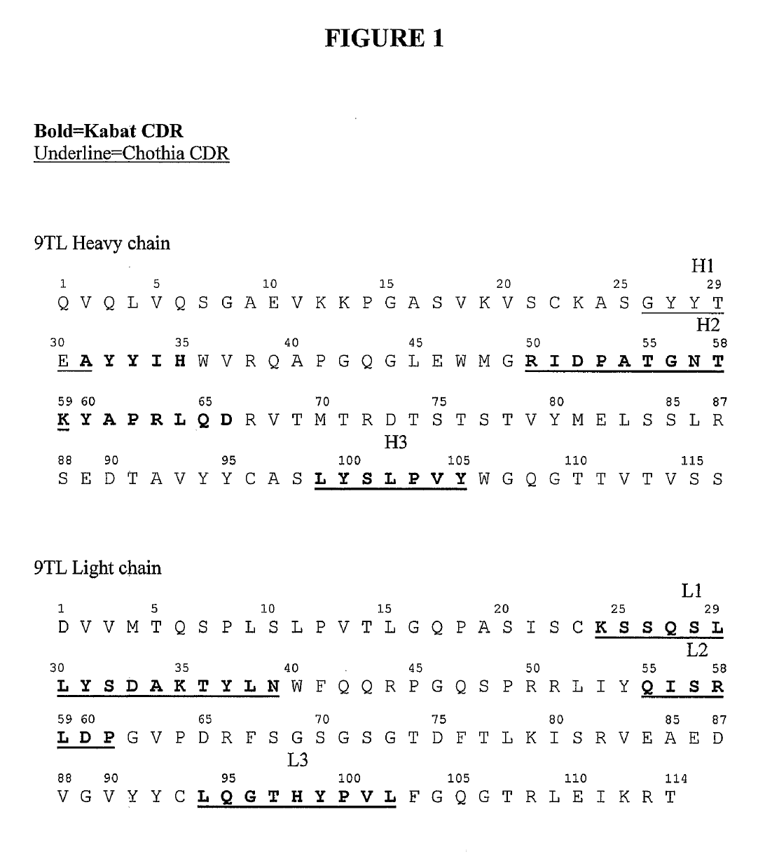

As used herein, the terms “9TL” and “antibody 9TL” are used interchangeably to refer to an antibody produced by expression vectors having deposit numbers of ATCC PTA-6124 and ATCC PTA-6125. The amino acid sequence of the heavy chain and light chain variable regions are shown in FIG. 1. The CDR portions of antibody 9TL (including Chothia and Kabat CDRs) are diagrammatically depicted in FIG. 1. The polynucleotides encoding the heavy and light chain variable regions are shown in SEQ ID NO:9 and SEQ ID NO:10. The characterization of 9TL is described in the Examples.

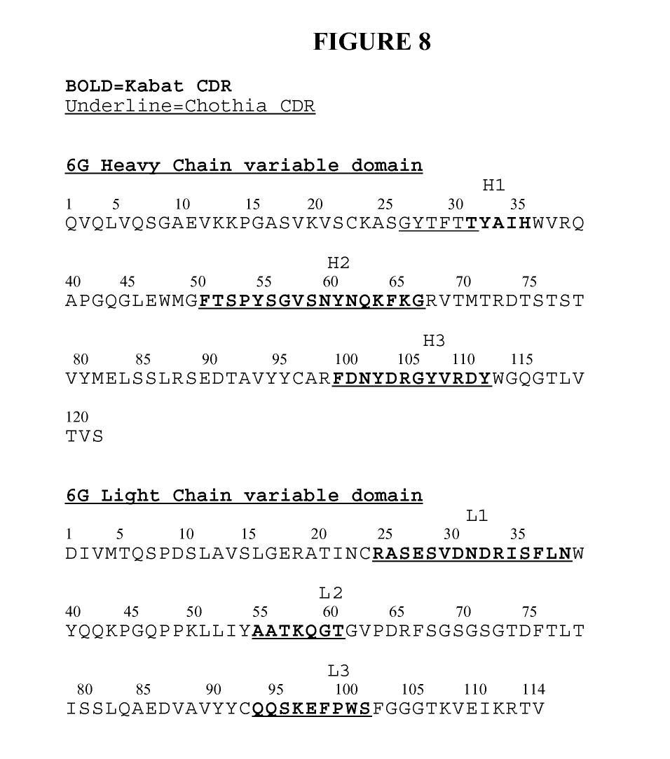

As used herein, the terms “6G” and “antibody 6G” are used interchangeably to refer to an antibody having the heavy chain amino acid sequence shown in SEQ ID NO:36 and the light chain amino acid sequence shown in SEQ ID NO:37. The amino acid sequence of the heavy chain and light chain variable regions are shown in FIG. 8. The CDR portions of antibody 6G (including Chothia and Kabat CDRs) are diagrammatically depicted in FIG. 8. The polynucleotides encoding the heavy and light chain are shown in SEQ ID NO:38 and SEQ ID NO:39. The characterization of 6G is described in the Examples.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

An epitope that “preferentially binds” or “specifically binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an Aβ₁₋₄₀ epitope is an antibody that binds this epitope with greater affinity, avidity, more readily, and/or with greater duration than it binds to other Aβ₁₋₄₀ epitopes or non-Aβ₁₋₄₀ epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably at least 90% pure, more preferably at least 95% pure, more preferably at least 98% pure, more preferably at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3.

As used herein, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202:163 (1996), may be performed.

A “functional Fc region” possesses at least one effector function of a native sequence Fc region. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95%, at least about 96%, at least about 97%, at least about 98%, at least about 99% sequence identity therewith.

As used herein “antibody-dependent cell-mediated cytotoxicity” and “ADCC” refer to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or U.S. Pat. No. 5,821,337. Useful effector cells for such assays include peripheral blood mononuclear cells (PBMC) and NK cells. Alternatively, or additionally, ADCC activity of the molecule of interest may be assessed in vivo, e.g., in a animal model such as that disclosed in Clynes et al., 1998, PNAS (USA), 95:652-656.

As used herein, an “effective dosage” or “effective amount” drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include but are not limited clinical results such as protecting or recovery of retinal function or preservation or restoration of visual acuity. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “treatment” or “treating” is an approach for obtaining beneficial or desired results including clinical results. For purposes of this invention, beneficial or desired clinical results include, but are not limited to, restoring, preventing or protecting retinal function.

By ‘biological effect of Aβ peptide’ or ‘Aβ biological activity’ is meant the effect of Aβ in ophthalmic diseases, which may be direct or indirect, and includes, without being bound by theory, the involvement of Aβ in lipid transport dysregulation. The indirect effect includes, but is not limited to Aβ having an effect on retina function and visual acuity.

As used herein, “delaying” development of ophthalmic diseases means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of the ophthalmic disease is a method that reduces probability of disease development in a given time frame and/or reduces extent of the disease in a given time frame, when compared to not using the method. Such comparisons are typically based on clinical studies, using a statistically significant number of subjects.

“Development” of ophthalmic diseases means the onset and/or progression of ophthalmic disease within an individual. Development of an ophthalmic disease can be detectable using standard clinical techniques as described herein. However, development also refers to disease progression that may be initially undetectable. For purposes of this invention, progression refers to the biological course of the disease state, in this case, as determined by standard opthalmogical examination or by more specialized testing. A variety of diagnostics tests include, but are not limited to, visual field, visual acuity, fluorescein angiography, electroretinograms, optical coherence tomography (OCT), visual evoked potentials (VEP), indocyanine green, color vision, Amsler grid, intraocular pressure and other diagnostic tools known to a person skilled in the art. Diagnostic tests for AMD include but are not limited to visual acuity, fundoscopic examination, fluorescein angiography, indocyanine green, and ocular coherence tomography (OCT) among others. “Development” includes occurrence, recurrence and onset. As used herein “onset” or “occurrence” of ophthalmic disease includes initial onset and/or recurrence.

As used herein, “protecting” or “protection” of retinal function refers to stabilizing or preserving retinal function. As used herein “recovery” of retinal function refers to restoration of retinal function after prior impairment. The protection or recovery of retinal function can be determined by measuring for statistically significant results (i.e. p<0.05), as measured by any of the above-mentioned ophthalmic diagnostic tools such as visual acuity, electroretinograms, visual field, fundoscopic examination, fluorescein angiography, indocyanine green, and ocular coherence tomography (OCT) among others. For example, as shown in Example 4 below, statistically significant protection or recovery of retinal function was shown by recovery of b-wave amplitude in electroretinograms (p=0.008).

“Preservation” or “restoration” of visual acuity can be measured by standard eye charts as well as a variety of ophthalmic diagnostic tools well known in the art.

As used herein, administration “in conjunction” includes simultaneous administration and/or administration at different times. Administration in conjunction also encompasses administration as a co-formulation or administration as separate compositions. As used herein, administration in conjunction is meant to encompass any circumstance wherein an anti-Aβ antibody and another agent are administered to an individual, which can occur simultaneously and/or separately. As further discussed herein, it is understood that an anti-Aβ antibody and the other agent can be administered at different dosing frequencies or intervals. For example, an anti-Aβ antibody can be administered weekly, while the other agent can be administered less frequently. It is understood that the anti-Aβ antibody and the other agent can be administered using the same route of administration or different routes of administration.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples.

An “individual” (alternatively referred to as a “subject”) is a mammal, more preferably a human. Mammals also include, but are not limited to, farm animals (such as cows), sport animals, pets (such as cats, dogs, horses), primates, mice and rats.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “kₒₙ”, as used herein, is intended to refer to the on rate constant for association of an antibody to an antigen.

The term “k_{off}”, as used herein, is intended to refer to the off rate constant for dissociation of an antibody from the antibody/antigen complex.

The term “K_{D}”, as used herein, is intended to refer to the equilibrium dissociation constant of an antibody-antigen interaction.

### Compositions and Methods of Making the Compositions

### Anti-Aβ Antibodies and Polypeptides:

### I. Antibody 9TL and 9TL Derived Antibodies and Polypeptides

This invention encompasses compositions, including pharmaceutical compositions, comprising antibody 9TL and its variants shown in Table 3 or polypeptide derived from antibody 9TL and its variants shown in Table 3; and polynucleotides comprising sequences encoding 9TL antibody and its variants or the polypeptide. As used herein, compositions comprise one or more antibodies or polypeptides (which may or may not be an antibody) that bind to C-terminus of Aβ₁₋₄₀, and/or one or more polynucleotides comprising sequences encoding one or more antibodies or polypeptides that bind to C-terminus of Aβ₁₋₄₀. These compositions may further comprise suitable excipients, such as pharmaceutically acceptable excipients including buffers, which are well known in the art. The antibodies and polypeptides of the invention are characterized by any (one or more) of the following characteristics: (a) binds to C-terminal peptide 28-40 of Aβ₁₋₄₀, but does not significantly bind to Aβ1-42 or Aβ1-43; (b) binds to C-terminal peptide 33-40 of Aβ₁₋₄₀; (c) suppresses formation of amyloid plaques in a subject; (d) reduces amyloid plaques in the eye of a subject; (e) treats, prevents, ameliorates one or more symptoms of ophthalmic disease, including but not limited age-related macular degeneration (both dry and wet), glaucoma, diabetic retinopathy (including macular edema) and other related retinal degenerative diseases; (f) causes significant protection or recovery of retinal function; and (g) causes significant preservation or restoration of visual acuity.

The antibodies and polypeptides of the invention may also exhibit a desirable safety profile in contrast to other reported anti-Aβ antibodies.

Accordingly, the invention provides any of the following, or compositions (including pharmaceutical compositions) comprising any of the following: (a) antibody 9TL or its variants shown in Table 3; (b) a fragment or a region of antibody 9TL or its variants shown in Table 3; (c) a light chain of antibody 9TL or its variants shown in Table 3; (d) a heavy chain of antibody 9TL or its variants shown in Table 3; (e) one or more variable region(s) from a light chain and/or a heavy chain of antibody 9TL or its variants shown in Table 3; (f) one or more CDR(s) (one, two, three, four, five or six CDRs) of antibody 9TL or its variants shown in Table 3; (g) CDR H3 from the heavy chain of antibody 9TL; (h) CDR L3 from the light chain of antibody 9TL or its variants shown in Table 3; (i) three CDRs from the light chain of antibody 9TL or its variants shown in Table 3; (j) three CDRs from the heavy chain of antibody 9TL or its variants shown in Table 3; (k) three CDRs from the light chain and three CDRs from the heavy chain, of antibody 9TL or its variants shown in Table 3; and (l) an antibody comprising any one of (b) through (k). The invention also provides polypeptides comprising any one or more of the above.

The CDR portions of antibody 9TL (including Chothia and Kabat CDRs) are diagrammatically depicted in FIG. 1. Determination of CDR regions is well within the skill of the art. It is understood that in some embodiments, CDRs can be a combination of the Kabat and Chothia CDR (also termed “combined CDRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof.

In some embodiments, the invention provides a polypeptide (which may or may not be an antibody) which comprises at least one CDR, at least two, at least three, or at least four, at least five, or all six CDRs that are substantially identical to at least one CDR, at least two, at least three, at least four, at least five or all six CDRs of 9TL or its variants shown in Table 3. Other embodiments include antibodies which have at least two, three, four, five, or six CDR(s) that are substantially identical to at least two, three, four, five or six CDRs of 9TL or derived from 9TL. In some embodiments, the at least one, two, three, four, five, or six CDR(s) are at least about 85%, 86%, 87%, 88%, 89%, 90%, 95%, 96%, 97%, 98%, or 99% identical to at least one, two, three, four, five or six CDRs of 9TL or its variants shown in Table 3. It is understood that, for purposes of this invention, binding specificity and/or overall activity is generally retained, although the extent of activity may vary compared to 9TL or its variants shown in Table 3 (may be greater or lesser).

The invention also provides a polypeptide (which may or may not be an antibody) which comprises an amino acid sequence of 9TL or its variants shown in Table 3 that has any of the following: at least 5 contiguous amino acids, at least 8 contiguous amino acids, at least about 10 contiguous amino acids, at least about 15 contiguous amino acids, at least about 20 contiguous amino acids, at least about 25 contiguous amino acids, at least about 30 contiguous amino acids of a sequence of 9TL or its variants shown in Table 3, wherein at least 3 of the amino acids are from a variable region of 9TL (FIG. 1) or its variants shown in Table 3. In one embodiment, the variable region is from a light chain of 9TL. In another embodiment, the variable region is from a heavy chain of 9TL. An exemplary polypeptide has contiguous amino acid (lengths described above) from both the heavy and light chain variable regions of 9TL. In another embodiment, the 5 (or more) contiguous amino acids are from a complementarity determining region (CDR) of 9TL shown in FIG. 1. In some embodiments, the contiguous amino acids are from a variable region of 9TL.

### II. Antibody 6G and 6G-Derived Antibodies and Polypeptides

The present invention further provides methods of treating an ophthalmic disease comprising administering an antibody or a polypeptide that binds to Aβ₁₋₄₀, Aβ₁₋₄₂, and Aβ₁₋₄₃. In some embodiments, the antibody or the polypeptide binds to Aβ₁₋₄₀ with higher affinity than its binding to Aβ₁₋₄₂, and Aβ₁₋₄₃. In some embodiments, the antibody binds to Aβ₁₋₃₆, Aβ₁₋₃₇, Aβ₁₋₃₈, and Aβ₁₋₃₉. In some embodiments, the antibody binds to Aβ₂₂₋₃₅. In some embodiments, the antibody binds to Aβ₂₈₋₄₀. In some embodiments, the antibody or the polypeptide binds to an epitope on Aβ₁₋₄₀ that includes amino acids 25-34 and 40.

This invention also provides methods of treating an ophthalmic disease comprising administering pharmaceutical compositions, comprising any of the antibodies or polypeptides described herein (such as antibody 6G and its variants shown in Table 8 or polypeptide derived from antibody 6G and its variants shown in Table 8); or polynucleotides described herein. As used herein, compositions comprise one or more antibodies or polypeptides (which may or may not be an antibody) that bind to C-terminus of Aβ₁₋₄₀, and/or one or more polynucleotides comprising sequences encoding one or more antibodies or polypeptides that bind to C-terminus of Aβ₁₋₄₀. These compositions may further comprise suitable excipients, such as pharmaceutically acceptable excipients including buffers, which are well known in the art.

The antibodies and polypeptides of the invention are characterized by any (one or more) of the following characteristics: (a) binds to Aβ₁₋₄₀, Aβ₁₋₄₂, and Aβ₁₋₄₃; (b) binds to Aβ₁₋₄₀, Aβ₁₋₄₂, and Aβ₁₋₄₃ with higher affinity binding to Aβ₁₋₄₀ than to Aβ₁₋₄₂ and Aβ₁₋₄₃; (c) binds to an epitope on Aβ₁₋₄₀ that includes amino acids 25-34 and 40; (d) binds to Aβ₁₋₃₆, Aβ₁₋₃₇, Aβ₁₋₃₈, and Aβ₁₋₃₉, but with lower affinity as compared to its binding to Aβ₁₋₄₀; (e) binds to Aβ₂₂₋₃₇ with a K_{D} of less than about 1 μM; (f) binds to Aβ₂₂₋₃₅; (g) binds to Aβ₂₈₋₄₀; (h) does not bind to APP expressed in a cell; (i) reduces amyloid plaques in the eye of a subject; (j) treats, prevents, ameliorates one or more symptoms of ophthalmic disease, including but not limited age-related macular degeneration (both dry and wet), glaucoma, diabetic retinopathy (including macular edema) and other related retinal degenerative diseases; (k) causes significant protection or recovery of retinal function; and (l) causes significant preservation or restoration of visual acuity. The antibodies and polypeptides of the invention may also have impaired effector function described herein. Antibodies and polypeptides having impaired effector function may exhibit a desirable safety profile in contrast to other reported anti-Aβ antibodies. For example, the compositions of the invention may not cause significant or unacceptable levels of any one or more of: bleeding in the brain vasculature (cerebral hemorrhage); meningoencephalitis (including changing magnetic resonance scan); elevated white blood count in cerebral spinal fluid; central nervous system inflammation.

Accordingly, the invention provides any of the following, or compositions (including pharmaceutical compositions) comprising any of the following: (a) antibody 6G or its variants shown in Table 8; (b) a fragment or a region of antibody 6G or its variants shown in Table 8; (c) a light chain of antibody 6G or its variants shown in Table 8; (d) a heavy chain of antibody 6G or its variants shown in Table 8; (e) one or more variable region(s) from a light chain and/or a heavy chain of antibody 6G or its variants shown in Table 8; (f) one or more CDR(s) (one, two, three, four, five or six CDRs) of antibody 6G or its variants shown in Table 8; (g) CDR H3 from the heavy chain of antibody 6G; (h) CDR L3 from the light chain of antibody 6G or its variants shown in Table 8; (i) three CDRs from the light chain of antibody 6G or its variants shown in Table 8; (j) three CDRs from the heavy chain of antibody 6G or its variants shown in Table 8; (k) three CDRs from the light chain and three CDRs from the heavy chain, of antibody 6G or its variants shown in Table 8; and (l) an antibody comprising any one of (b) through (k). The invention also provides polypeptides comprising any one or more of the above.

The CDR portions of antibody 6G (including Chothia and Kabat CDRs) are diagrammatically depicted in FIG. 8. Determination of CDR regions is well within the skill of the art.

In some embodiments, the invention provides a polypeptide (which may or may not be an antibody) which comprises at least one CDR, at least two, at least three, or at least four, at least five, or all six CDRs that are substantially identical to at least one CDR, at least two, at least three, at least four, at least five or all six CDRs of 6G or its variants shown in Table 8. Other embodiments include antibodies which have at least two, three, four, five, or six CDR(s) that are substantially identical to at least two, three, four, five or six CDRs of 6G or derived from 6G. In some embodiments, the at least one, two, three, four, five, or six CDR(s) are at least about 85%, 86%, 87%, 88%, 89%, 90%, 95%, 96%, 97%, 98%, or 99% identical to at least one, two, three, four, five or six CDRs of 6G or its variants shown in Table 8. It is understood that, for purposes of this invention, binding specificity and/or overall activity is generally retained, although the extent of activity may vary compared to 6G or its variants shown in Table 8 (may be greater or lesser).

The invention also provides a polypeptide (which may or may not be an antibody) which comprises an amino acid sequence of 6G or its variants shown in Table 8 that has any of the following: at least 5 contiguous amino acids, at least 8 contiguous amino acids, at least about 10 contiguous amino acids, at least about 15 contiguous amino acids, at least about 20 contiguous amino acids, at least about 25 contiguous amino acids, at least about 30 contiguous amino acids of a sequence of 6G or its variants shown in Table 8, wherein at least 3 of the amino acids are from a variable region of 6G (Figure) or its variants shown in Table 8. In one embodiment, the variable region is from a light chain of 6G. In another embodiment, the variable region is from a heavy chain of 6G. An exemplary polypeptide has contiguous amino acid (lengths described above) from both the heavy and light chain variable regions of 6G. In another embodiment, the 5 (or more) contiguous amino acids are from a complementarity determining region (CDR) of 6G shown in FIG. 8. In some embodiments, the contiguous amino acids are from a variable region of 6G.

The binding affinities of the antibodies and polypeptides of the invention may vary, and need not be (but can be) a particular value or range, as the exemplary embodiments described below. The binding affinity (K_{D}) of the antibodies and polypeptides of the invention to the Aβ peptide (including Aβ₁₋₄₀, Aβ₁₋₄₂ or Aβ₁₋₄₃ peptides) can be about 0.10 to about 0.80 nM, about 0.15 to about 0.75 nM and about 0.18 to about 0.72 nM. In some embodiments, the binding affinity is about 2 pM, about 5 pM, about 10 pM, about 15 pM, about 20 pM, about 40 pM, or greater than about 40 pM. In one embodiment, the binding affinity is between about 2 pM and 22 pM. In other embodiments, the binding affinity is less than about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM. In some embodiment, the binding affinity is about 10 nM. In other embodiments, the binding affinity is less than about 10 nM, less than about 50 nM, less than about 100 nM, less than about 150 nM, less than about 200 nM, less than about 250 nM, less than about 500 nM, or less than about 1000 nM. In other embodiments, the binding affinity is less than about 5 nM. In other embodiments, the binding affinity is less than about 1 nM. In other embodiments, the binding affinity is about 0.1 nM or about 0.07 nM. In other embodiments, the binding affinity is less than about 0.1 nM or less than about 0.07 nM. In other embodiments, the binding affinity is from any of about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM to any of about 2 pM, about 5 pM, about 10 pM, about 15 pM, about 20 pM, or about 40 pM. In some embodiments, the binding affinity is any of about 10 nM, about 5 nM, about 1 nM, about 900 pM, about 800 pM, bout 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM. In still other embodiments, the binding affinity is about 2 pM, about 5 pM, about 10 pM, about 15 pM, about 20 pM, about 40 pM, or greater than about 40 pM. The antibody or polypeptides of the invention can bind to a combination of the Aβ₁₋₄₀, Aβ₁₋₄₂ and/or Aβ₁₋₄₂ peptides. In one embodiment, the antibody or polypeptides bind to at least Aβ₁₋₄₀ and Aβ₁₋₄₂ peptides.

The antibodies and polypeptides of the invention may also bind to any one or more of Aβ₁₋₃₆, Aβ₁₋₃₇, Aβ₁₋₃₈, Aβ₁₋₃₉, Aβ₁₋₄₂, and Aβ₁₋₄₃, but in some embodiments the binding affinity to any one or more of these peptides is less than their binding affinities to Aβ₁₋₄₀. In some embodiments, the K_{D} of the antibodies or polypeptides to any one or more of Aβ₁₋₃₆, Aβ₁₋₃₇, Aβ₁₋₃₈, Aβ₁₋₃₉, Aβ₁₋₄₂, and Aβ₁₋₄₃ is at least about 5-fold, at least about 10-fold, at least about 20-fold, at least about 30-fold, at least about 40-fold, at least about 50-fold, at least about 80-fold, at least about 100-fold, at least about 150-fold, at least about 200-fold, or at least about 250-fold of the K_{D} to Aβ₁₋₄₀.

The invention also provides methods of making any of these antibodies or polypeptides. The antibodies of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

In another alternative, the antibodies can be made recombinantly using procedures that are well known in the art. In one embodiment, a polynucleotide comprises a sequence encoding the heavy chain and/or the light chain variable regions of the antibody. In another embodiment, the polynucleotide comprising the nucleotide sequence are cloned into one or more vectors for expression or propagation. The sequence encoding the antibody of interest may be maintained in a vector in a host cell and the host cell can then be expanded and frozen for future use. Vectors (including expression vectors) and host cells are further described herein.

The invention also encompasses single chain variable region fragments (“scFv”) of antibodies of this invention, such as 9TL and 6G. Single chain variable region fragments are made by linking light and/or heavy chain variable regions by using a short linking peptide. Bird et al. (1988) Science 242:423-426. An example of a linking peptide is (GGGGS)₃ which bridges approximately 3.5 nm between the carboxy terminus of one variable region and the amino terminus of the other variable region. Linkers of other sequences have been designed and used. Bird et al. (1988). Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

For example, bispecific antibodies, monoclonal antibodies that have binding specificities for at least two different antigens, i.e. Aβ₁₋₄₀ and Aβ₁₋₄₂. can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539).

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690, published Mar. 3, 1994.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents and techniques are well known in the art, and are described in U.S. Pat. No. 4,676,980.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Humanized antibody comprising one or more CDRs of antibody 9TL or one or more CDRs derived from antibody 9TL can be made using any methods known in the art. For example, four general steps may be used to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; 5,225,539; 6,548,640.

In the recombinant humanized antibodies, the Fc portion can be modified to avoid interaction with [symbol] receptor and the complement immune system. This type of modification was designed by Dr. Mike Clark from the Department of Pathology at Cambridge University, and techniques for preparation of such antibodies are described in WO 99/58572, published Nov. 18, 1999.

For example, the constant region may be engineered to more resemble human constant regions to avoid immune response if the antibody for use in clinical trials and treatments in humans. See, for example, U.S. Pat. Nos. 5,997,867 and 5,866,692.

The invention encompasses modifications to antibody 9TL and 6G, including functionally equivalent antibodies which do not significantly affect their properties and variants which have enhanced or decreased activity and/or affinity. For example, the amino acid sequence of antibody 9TL or 6G may be mutated to obtain an antibody with the desired binding affinity to the target Aβ peptide. Modification of polypeptides is routine practice in the art and need not be described in detail herein. Modification of polypeptides is exemplified in the Examples. Examples of modified polypeptides include polypeptides with conservative substitutions of amino acid residues, one or more deletions or additions of amino acids which do not significantly deleteriously change the functional activity, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the serum half-life of the antibody.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 1 under the heading of “conservative substitutions”. If such substitutions result in a change in biological activity, then more substantial changes, denominated “exemplary substitutions” in Table 1, or as further described below in reference to amino acid classes, may be introduced and the products screened.

TABLE 1
Amino Acid Substitutions

| Original | Conservative | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; Ala; |
| | | Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) Non-polar: Norleucine, Met, Ala, Val, Leu, Ile;

(2) Polar without charge: Cys, Ser, Thr, Asn, Gln;

(3) Acidic (negatively charged): Asp, Glu;

(4) Basic (positively charged): Lys, Arg;

(5) Residues that influence chain orientation: Gly, Pro; and

(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment. Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. In still other embodiments, the CDR domain is CDR H3 and/or CDR L3.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Hefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)—N-acetylglucosaminyltransferase III (GnTIII), a glycosyltransferase catalyzing formation of bisecting GlcNAc, was reported to have improved ADCC activity (Umana et al., 1999, Mature Biotech. 17:176-180).

lycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5.278,299). Glycosylation, or certain types of glycosylation, can be enzymatically removed from the glycoprotein, for example using endoglycosidase H (Endo H), N-glycosidase F as described in Example 3, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified 9TL polypeptides are made using established procedures in the art and can be screened using standard assays known in the art, some of which are described below and in the Examples.

In some embodiments of the invention, the antibody comprises a modified constant region, such as a constant region that is immunologically inert or partially inert, e.g., does not trigger complement mediated lysis, does not stimulate antibody-dependent cell mediated cytotoxicity (ADCC), or does not activate microglia; or have reduced activities (compared to the unmodified antibody) in any one or more of the following: triggering complement mediated lysis, stimulating antibody-dependent cell mediated cytotoxicity (ADCC), or activating microglia. Different modifications of the constant region may be used to achieve optimal level and/or combination of effector functions. See, for example, Morgan et al., Immunology 86:319-324 (1995); Lund et al., J. Immunology 157:4963-9 157:4963-4969 (1996); Idusogie et al., J. Immunology 164:4178-4184 (2000); Tao et al., J. Immunology 143: 2595-2601 (1989); and Jefferis et al., Immunological Reviews 163:59-76 (1998). In some embodiments, the constant region is modified as described in Eur. J. Immunol. (1999) 29:2613-2624; PCT Application No. PCT/GB99/01441; and/or UK Patent Application No. 9809951.8. In other embodiments, the antibody comprises a human heavy chain IgG2a constant region comprising the following mutations: A330P331 to S330S331 (amino acid numbering with reference to the wildtype IgG2a sequence). Eur. J. Immunol. (1999) 29:2613-2624. In still other embodiments, the constant region is aglycosylated for N-linked glycosylation. In some embodiments, the constant region is aglycosylated for N-linked glycosylation by mutating the glycosylated amino acid residue or flanking residues that are part of the N-glycosylation recognition sequence in the constant region. For example, N-glycosylation site N297 may be mutated to A, Q, K, or H. See, Tao et al., J. Immunology 143: 2595-2601 (1989); and Jefferis et al., Immunological Reviews 163:59-76 (1998). In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications include antibodies that have been modified as described in PCT Publication No. WO 99/58572, published Nov. 18, 1999. These antibodies comprise, in addition to a binding domain directed at the target molecule, an effector domain having an amino acid sequence substantially homologous to all or part of a constant domain of a human immunoglobulin heavy chain. These antibodies are capable of binding the target molecule without triggering significant complement dependent lysis, or cell-mediated destruction of the target. In some embodiments, the effector domain is capable of specifically binding FcRn and/or FcγRIIb. These are typically based on chimeric domains derived from two or more human immunoglobulin heavy chain C_{H}2 domains. Antibodies modified in this manner are particularly suitable for use in chronic antibody therapy, to avoid inflammatory and other adverse reactions to conventional antibody therapy.

The invention includes affinity matured embodiments. For example, affinity matured antibodies can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al, 1992, J. Mol. Biol., 226:889-896; and WO2004/058184).

The following methods may be used for adjusting the affinity of an antibody and for characterizing a CDR. One way of characterizing a CDR of an antibody and/or altering (such as improving) the binding affinity of a polypeptide, such as an antibody, termed “library scanning mutagenesis”. Generally, library scanning mutagenesis works as follows. One or more amino acid positions in the CDR are replaced with two or more (such as 3, 4, 5, 6, 7, 8, 9, 10, 11, 12, 13, 14, 15, 16, 17, 18, 19, or 20) amino acids using art recognized methods. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased or no binding are identified. Methods for determining binding affinity are well-known in the art. Binding affinity may be determined using BIAcore surface plasmon resonance analysis, which detects differences in binding affinity of about 2-fold or greater. BIAcore is particularly useful when the starting antibody already binds with a relatively high affinity, for example a K_{D} of about 10 nM or lower. Screening using BIAcore surface plasmon resonance is described in the Examples, herein.

Binding affinity may be determined using Kinexa Biocensor, scintillation proximity assays, ELISA, ORIGEN immunoassay (IGEN), fluorescence quenching, fluorescence transfer, and/or yeast display. Binding affinity may also be screened using a suitable bioassay.

In some embodiments, every amino acid position in a CDR is replaced (in some embodiments, one at a time) with all 20 natural amino acids using art recognized mutagenesis methods (some of which are described herein). This generates small libraries of clones (in some embodiments, one for every amino acid position that is analyzed), each with a complexity of 20 members (if all 20 amino acids are substituted at every position).

In some embodiments, the library to be screened comprises substitutions in two or more positions, which may be in the same CDR or in two or more CDRs. Thus, the library may comprise substitutions in two or more positions in one CDR. The library may comprise substitution in two or more positions in two or more CDRs. The library may comprise substitution in 3, 4, 5, or more positions, said positions found in two, three, four, five or six CDRs. The substitution may be prepared using low redundancy codons. See, e.g., Table 2 of Balint et al., (1993) Gene 137(1):109-18).

The CDR may be CDRH3 and/or CDRL3. The CDR may be one or more of CDRL1, CDRL2, CDRL3, CDRH1, CDRH2, and/or CDRH3. The CDR may be a Kabat CDR, a Chothia CDR, or an extended CDR. Candidates with improved binding may be sequenced, thereby identifying a CDR substitution mutant which results in improved affinity (also termed an “improved” substitution). Candidates that bind may also be sequenced, thereby identifying a CDR substitution which retains binding.

Multiple rounds of screening may be conducted. For example, candidates (each comprising an amino acid substitution at one or more position of one or more CDR) with improved binding are also useful for the design of a second library containing at least the original and substituted amino acid at each improved CDR position (i.e., amino acid position in the CDR at which a substitution mutant showed improved binding). Preparation, and screening or selection of this library is discussed further below.

Library scanning mutagenesis also provides a means for characterizing a CDR, in so far as the frequency of clones with improved binding, the same binding, decreased binding or no binding also provide information relating to the importance of each amino acid position for the stability of the antibody-antigen complex. For example, if a position of the CDR retains binding when changed to all 20 amino acids, that position is identified as a position that is unlikely to be required for antigen binding. Conversely, if a position of CDR retains binding in only a small percentage of substitutions, that position is identified as a position that is important to CDR function. Thus, the library scanning mutagenesis methods generate information regarding positions in the CDRs that can be changed to many different amino acid (including all 20 amino acids), and positions in the CDRs which cannot be changed or which can only be changed to a few amino acids.

Candidates with improved affinity may be combined in a second library, which includes the improved amino acid, the original amino acid at that position, and may further include additional substitutions at that position, depending on the complexity of the library that is desired, or permitted using the desired screening or selection method. In addition, if desired, adjacent amino acid position can be randomized to at least two or more amino acids. Randomization of adjacent amino acids may permit additional conformational flexibility in the mutant CDR, which may in turn, permit or facilitate the introduction of a larger number of improving mutations. The library may also comprise substitution at positions that did not show improved affinity in the first round of screening.

The second library is screened or selected for library members with improved and/or altered binding affinity using any method known in the art, including screening using BIAcore surface plasmon resonance analysis, and selection using any method known in the art for selection, including phage display, yeast display, and ribosome display.

The invention also encompasses fusion proteins comprising one or more fragments or regions from the antibodies (such as 9TL and 6G) or polypeptides of this invention. In one embodiment, a fusion polypeptide is provided that comprises at least 10 contiguous amino acids of the variable light chain region sand/or at least 10 amino acids of the variable heavy chain region shown. In other embodiments, a fusion polypeptide is provided that comprises at least about 10, at least about 15, at least about 20, at least about 25, or at least about 30 contiguous amino acids of the variable light chain region; and/or at least about 10, at least about 15, at least about 20, at least about 25, or at least about 30 contiguous amino acids of the variable heavy chain region. In another embodiment, the fusion polypeptide comprises a light chain variable region and/or a heavy chain variable region of 9TL or 6G. In another embodiment, the fusion polypeptide comprises one or more CDR(s) of 9TL or 6G. In still other embodiments, the fusion polypeptide comprises CDR H3 and/or CDR L3 of antibody 9TL or 6G. For purposes of this invention, an 9TL or 6G fusion protein contains one or more 9TL or 6G antibodies, respectively and another amino acid sequence to which it is not attached in the native molecule, for example, a heterologous sequence or a homologous sequence from another region. Exemplary heterologous sequences include, but are not limited to a “tag” such as a FLAG tag or a 6H is tag. Tags are well known in the art.

A fusion polypeptide can be created by methods known in the art, for example, synthetically or recombinantly. Typically, the fusion proteins of this invention are made by preparing an expressing a polynucleotide encoding them using recombinant methods described herein, although they may also be prepared by other means known in the art, including, for example, chemical synthesis.

This invention also provides compositions comprising antibodies or polypeptides conjugated (for example, linked) to an agent that facilitate coupling to a solid support (such as biotin or avidin). For simplicity, reference will be made generally to antibodies with the understanding that these methods apply to any of the Aβ binding embodiments described herein. Conjugation generally refers to linking these components as described herein. The linking (which is generally fixing these components in proximate association at least for administration) can be achieved in any number of ways. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

An antibody or polypeptide of this invention may be linked to a labeling agent (alternatively termed “label”) such as a fluorescent molecule, a radioactive molecule or any others labels known in the art. Labels are known in the art which generally provide (either directly or indirectly) a signal. The invention also provides compositions (including pharmaceutical compositions) and kits comprising antibody 9TL or 6G, and, as this disclosure makes clear, any or all of the antibodies and/or polypeptides described herein.

Anti-Aβ Peptide Antibodies and Polypeptides Having Impaired Effector Function

The methods of the invention use antibodies or polypeptides (including pharmaceutical compositions comprising the antibodies or polypeptides) that specifically bind to a β-amyloid (Aβ) peptide and have impaired effector function. The antibodies and polypeptides are further characterized by any (one or more) of the following characteristics: (a) suppresses formation of amyloid plaques in a subject; (b) reduces amyloid plaques in the eye of a subject; (c) treats, prevents, ameliorates one or more symptoms of ophthalmic disease, including but not limited age-related macular degeneration (both dry and wet), glaucoma, diabetic retinopathy (including macular edema) and other related retinal degenerative diseases; (d) causes significant protection or recovery of retinal function; and (e) causes significant preservation or restoration of visual acuity.

The antibodies and polypeptides described herein may exhibit a desirable safety profile, for example, the compositions of the invention do not cause significant or unacceptable levels or have a reduced level of any one or more of: bleeding in the brain vasculature (cerebral hemorrhage); meningoencephalitis (including changing magnetic resonance scan); elevated white blood count in cerebral spinal fluid; central nervous system inflammation.

As used herein, an antibody or a polypeptide having an “impaired effector function” (used interchangeably with “immunologically inert” or “partially immunologically inert”) refers to antibodies or polypeptides that do not have any effector function or have reduced activity or activities of effector function (compared to antibody or polypeptide having an unmodified or a naturally occurring constant region), e.g., having no activity or reduced activity in any one or more of the following: a) triggering complement mediated lysis; b) stimulating antibody-dependent cell mediated cytotoxicity (ADCC); and c) activating microglia. The effector function activity may be reduced by about any of 10%, 20%, 30%, 40%, 50%, 60%, 70%, 80%, 90%, 95%, 99%, and 100%. In some embodiments, the antibody binds to a beta-amyloid peptide without triggering significant complement dependent lysis, or cell mediated destruction of the target. For example, the Fc receptor binding site on the constant region may be modified or mutated to remove or reduce binding affinity to certain Fc receptors, such as FcγRI, FcγRII, and/or FcγRIII. For simplicity, reference will be made to antibodies with the understanding that embodiments also apply to polypeptides. EU numbering system (Kabat et al., Sequences of Proteins of Immunological Interest; 5th ed. Public Health Service, National Institutes of Healthy, Bethesda, Md., 1991) is used to indicate which amino acid residue(s) of the constant region (e.g., of an IgG antibody) are altered or mutated. The numbering may be used for a specific type of antibody (e.g., IgG1) or a species (e.g., human) with the understanding that similar changes can be made across types of antibodies and species.

In some embodiments, the antibody that specifically binds to the an Aβ peptide comprises a heavy chain constant region having impaired effector function. The heavy chain constant region may have naturally occurring sequence or is a variant. In some embodiments, the amino acid sequence of a naturally occurring heavy chain constant region is mutated, e.g., by amino acid substitution, insertion and/or deletion, whereby the effector function of the constant region is impaired. In some embodiments, the N-glycosylation of the Fc region of a heavy chain constant region may also be changed, e.g., may be removed completely or partially, whereby the effector function of the constant region is impaired.

In some embodiments, the effector function is impaired by removing N-glycosylation of the Fc region (e.g., in the CH 2 domain of IgG) of the anti-Aβ peptide. In some embodiments, N-glycosylation of the Fc region is removed by mutating the glycosylated amino acid residue or flanking residues that are part of the glycosylation recognition sequence in the constant region. The tripeptide sequences asparagine-X-serine (N—X—S), asparagine-X-threonine (N—X-T) and asparagine-X-cysteine (N—X—C), where X is any amino acid except proline, are the recognition sequences for enzymatic attachment of the carbohydrate moiety to the asparagine side chain for N-glycosylation. Mutating any of the amino acid in the tripeptide sequences in the constant region yields an aglycosylated IgG. For example, N-glycosylation site N297 of human IgG1 and IgG3 may be mutated to A, D, Q, K, or H. See, Tao et al., J. Immunology 143: 2595-2601 (1989); and Jefferis et al., Immunological Reviews 163:59-76 (1998). It has been reported that human IgG1 and IgG3 with substitution of Asn-297 with Gln, His, or Lys do not bind to the human FcγRI and do not activate complement with C1q binding ability completely lost for IgG1 and dramatically decreased for IgG3. In some embodiments, the amino acid N in the tripeptide sequences is mutated to any one of amino acid A, C, D, E, F, G, H, I, K, L, M, P, Q, R, S, T, V, W, Y. In some embodiments, the amino acid N in the tripeptide sequences is mutated to a conservative substitution. In some embodiments, the amino acid X in the tripeptide sequences is mutated to proline. In some embodiments, the amino acid S in the tripeptide sequences is mutated to A, D, E, F, G, H, I, K, L, M, N, P, Q, R, V, W, Y. In some embodiments, the amino acid T in the tripeptide sequences is mutated to A, D, E, F, G, H, I, K, L, M, N, P, Q, R, V, W, Y. In some embodiments, the amino acid C in the tripeptide sequences is mutated to A, D, E, F, G, H, I, K, L, M, N, P, Q, R, V, W, Y. In some embodiments, the amino acid following the tripeptide is mutated to P. In some embodiments, the N-glycosylation in the constant region is removed enzymatically (such as N-glycosidase F as described in Example 3, endoglycosidase F1, endoglycosidase F2, endoglycosidase F3, and englycosidase H). Removing N-glycosylation may also be achieved by producing the antibody in a cell line having deficiency for N-glycosylation. Wright et al., J. Immunol. 160(7):3393-402 (1998).

In some embodiments, amino acid residue interacting with oligosaccharide attached to the N-glycosylation site of the constant region is mutated to reduce binding affinity to FcγRI. For example, F241, V264, D265 of human IgG3 may be mutated. See, Lund et al., J. Immunology 157:4963-4969 (1996). In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000). Antibodies described in PCT WO 99/58572 and Armour et al. comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain has a reduced affinity for FcγRI, FcγRIIa, and FcγRIII. In some embodiments, the effector domain is capable of specifically binding FcRn and/or FcγRIIb. These are typically based on chimeric domains derived from two or more human immunoglobulin heavy chain C_{H}2 domains. Antibodies modified in this manner are particularly suitable for use in chronic antibody therapy, to avoid inflammatory and other adverse reactions to conventional antibody therapy. In some embodiments, the heavy chain constant region of the antibody is a human heavy chain IgG1 with any of the following mutations: 1) A327A330P331 to G327S330S331; 2) E233L234L235G236 to P233V234A235 with G236 deleted; 3) E233L234L235 to P233V234A235; 4) E233L234L235G236A327A330P331 to P233V234A235G327S330S331 with G236 deleted; 5) E233L234L235A327A330P331 to P233V234A235G327S330S331; and 6) N297 to A297 or any other amino acid except N. In some embodiments, the heavy chain constant region of the antibody is a human heavy chain IgG2 with the following mutations: A330P331 to S330S331. In some embodiments, the heavy chain constant region of the antibody is a human heavy chain IgG4 with any of the following mutations: E233F234L235G236 to P233V234A235 with G236 deleted; E233F234L235 to P233V234A235; and S228L235 to P228E235.

The constant region of the antibodies may also be modified to impair complement activation. For example, complement activation of IgG antibodies following binding of the C1 component of complement may be reduced by mutating amino acid residues in the constant region in a C1 binding motif (e.g., C1q binding motif). It has been reported that Ala mutation for each of D270, K322, P329, P331 of human IgG1 significantly reduced the ability of the antibody to bind to C1q and activating complement. For murine IgG2b, C1q binding motif constitutes residues E318, K320, and K322. Idusogie et al., J. Immunology 164:4178-4184 (2000); Duncan et al., Nature 322: 738-740 (1988).

Clq binding motif E318, K320, and K322 identified for murine IgG2b is believed to be common for other antibody isotypes. Duncan et al., Nature 322: 738-740 (1988). C1q binding activity for IgG2b can be abolished by replacing any one of the three specified residues with a residue having an inappropriate functionality on its side chain. It is not necessary to replace the ionic residues only with Ala to abolish C1q binding. It is also possible to use other alkyl-substituted non-ionic residues, such as Gly, Ile, Leu, or Val, or such aromatic non-polar residues as Phe, Tyr, Trp and Pro in place of any one of the three residues in order to abolish C1q binding. In addition, it is also be possible to use such polar non-ionic residues as Ser, Thr, Cys, and Met in place of residues 320 and 322, but not 318, in order to abolish C1q binding activity.

The invention also provides antibodies having impaired effector function wherein the antibody has a modified hinge region. Binding affinity of human IgG for its Fc receptors can be modulated by modifying the hinge region. Canfield et al., J. Exp. Med. 173:1483-1491 (1991); Hezareh et al., J. Virol. 75:12161-12168 (2001); Redpath et al., Human Immunology 59:720-727 (1998). Specific amino acid residues may be mutated or deleted. The modified hinge region may comprise a complete hinge region derived from an antibody of different antibody class or subclass from that of the CH1 domain. For example, the constant domain (CH1) of a class IgG antibody can be attached to a hinge region of a class IgG4 antibody. Alternatively, the new hinge region may comprise part of a natural hinge or a repeating unit in which each unit in the repeat is derived from a natural hinge region. In some embodiments, the natural hinge region is altered by converting one or more cysteine residues into a neutral residue, such as alanine, or by converting suitably placed residues into cysteine residues. U.S. Pat. No. 5,677,425. Such alterations are carried out using art recognized protein chemistry and, preferably, genetic engineering techniques and as described herein.

Polypeptides that specifically bind to an Aβ peptide and fused to a heavy chain constant region having impaired effector function may also be used for the methods described herein. In some embodiments, the polypeptide comprises a sequence derived from antibody 9TL or its variants shown in Table 3. In some embodiments, the polypeptide is derived from a single domain antibody that binds to an Aβ peptide. Single domain antibodies can be generated using methods known in the art. Omidfar et al., Tumour Biol. 25:296-305 (2004); Herring et al., Trends in Biotechnology 21:484-489 (2003).

In some embodiments, the antibody or polypeptide is not a F(ab′)₂ fragment. In some embodiments, the antibody or polypeptide is not a Fab fragment. In some embodiments, the antibody or polypeptide is not a single chain antibody scFv. In some embodiments, the antibody or polypeptide is a PEGylated F(ab′)₂ fragment. In some embodiments, the antibody or polypeptide is a PEGylated Fab fragment. In some embodiments, the antibody or polypeptide is a PEGylated single chain antibody scFv.

Other methods to make antibodies having impaired effector function known in the art may also be used.

Antibodies and polypeptides with modified constant regions can be tested in one or more assays to evaluate level of effector function reduction in biological activity compared to the starting antibody. For example, the ability of the antibody or polypeptide with an altered Fc region to bind complement or Fc receptors (for example, Fc receptors on microglia), or altered hinge region can be assessed using the assays disclosed herein as well as any art recognized assay. PCT WO 99/58572; Armour et al., Molecular Immunology 40: 585-593 (2003); Reddy et al., J. Immunology 164:1925-1933 (2000); Song et al., Infection and Immunity 70:5177-5184 (2002).

In some embodiments, the antibody that specifically binds to β-amyloid peptide is a polyclonal antibody. In some embodiments, the antibody is a monoclonal antibody. In some embodiments, the antibody is a human antibody. In some embodiments, the antibody is a chimeric antibody. In some embodiments, the antibody is a humanized antibody. In some embodiments, the antibody is a primatized antibody. See, e.g., Yocum et al., J. Rheumatol. 25:1257-62 (1998); Bugelski et al., Human & Experimental Toxicoloy 19:230-243 (2000). In some embodiments, the antibody is deimmunized by mutation so that the antibody does not activate human immune system. See, e.g., Nanus, et al., J. Urology 170:S84-S89 (2003).

As used herein, Aβ peptide includes any fragments of the enzymatic cleavage products of amyloid precursor protein. For example, Aβ peptide includes any fragments of Aβ₁₋₄₀, Aβ₁₋₄₂, or Aβ₁₋₄₃; and peptides which are truncated with various number of amino acids at the N-terminus or the C-terminus of Aβ₁₋₄₀, Aβ₁₋₄₂, or Aβ₁₋₄₃. Amino acid numbering used herein is based on the numbering for Aβ1-43 (SEQ ID NO:17).

In some embodiments, the antibody or polypeptide specifically binds to an epitope within residues 1-16 of Aβ peptide. In some embodiments, the antibody or polypeptide specifically binds to an epitope within residues 16-28 of Aβ peptide. In some embodiments, the antibody or polypeptide specifically binds to an epitope within residues 28-40 of Aβ₁₋₄₀ peptide. In some embodiments, the antibody or polypeptide specifically binds to an epitope within residues 28-42 of Aβ₁₋₄₂ peptide. In some embodiments, the antibody or polypeptide specifically binds to an epitope within residues 28-43 of Aβ₁₋₄₃ peptide. In some embodiments, the antibody or polypeptide specifically binds to an Aβ peptide without binding to full-length amyloid precursor protein (APP). In some embodiments, the antibody or the polypeptide specifically binds to the aggregated form of Aβ without binding to the soluble form. In some embodiments, the antibody or the polypeptide specifically binds to the soluble form of Aβ without binding to the aggregated form. In some embodiments, the antibody or the polypeptide specifically binds to both aggregated form and soluble forms of Aβ. Antibodies that bind to various aggregated form of Aβ are known in the art, for example, antibodies that bind to amyloid beta-derived diffusible ligands (ADDLs); antibodies that bind to amyloid fibrils and/or deposit. WO 03/104437; U.S. Pub. No. 2003/0147887; U.S. Pub. No. 2004/0219146.

In some embodiments, the antibody or polypeptide comprises one, two, or three CDRs from the 3D6 immunoglobulin light chain (SEQ ID NO:2 in U.S. Pub. Nos. 2003/0165496, or 2004/0087777), and/or one, two, or three CDRs from the 3D6 immunoglobulin heavy chain (SEQ ID NO:4 in U.S. Pub. Nos. 2003/0165496, or 2004/0087777). In some embodiments, the antibody or polypeptide comprises a variable heavy chain region as set forth in SEQ ID NO:8 in U.S. Pub. No. 2003/0165496 and a variable light chain region as set forth in SEQ ID NO:5 in U.S. Pub. No. 2003/0165496. In some embodiments, the antibody or polypeptide comprises a variable heavy chain region as set forth in SEQ ID NO:12 in U.S. Pub. No. 2003/0165496 and a variable light chain region as set forth in SEQ ID NO:11 in U.S. Pub. No. 2003/0165496. In some embodiments, the antibody or polypeptide comprises one, two, or three CDRs from the 10D5 immunoglobulin light chain (SEQ ID NO:14 in U.S. Pub. Nos. 2003/0165496, or 2004/0087777), and/or one, two, or three CDRs from the 10D5 immunoglobulin heavy chain (SEQ ID NO:16 in U.S. Pub. Nos. 2003/0165496, or 2004/0087777).

In some embodiments, the antibody or polypeptide specifically binds to an epitope within residues 33-40 of Aβ₁₋₄₀. In some embodiments, the antibody or polypeptide specifically binds to an epitope on Aβ₁₋₄₀ that includes amino acid 35-40. In some embodiments, the antibody or polypeptide specifically binds to an epitope on Aβ₁₋₄₀ that includes amino acid 36-40. In some embodiments, the antibody or polypeptide specifically binds to an epitope on Aβ₁₋₄₀ that includes amino acid 39 and/or 40. In some embodiments, the antibody or polypeptide specifically binds to Aβ₁₋₄₀ but does not specifically bind to Aβ₁₋₄₂ and/or Aβ₁₋₄₃. In some embodiments, the antibody or polypeptide is antibody 9TL or an antibody or a polypeptide derived from 9TL described herein. In some embodiments, the antibody or polypeptide competitively inhibits binding of antibody 9TL and/or antibody or polypeptide derived from 9TL to Aβ₁₋₄₀. In some embodiments, the antibody is not antibody 2286 described in PCT WO 2004/032868. In other embodiments, the antibody or polypeptide is antibody 6G or an antibody or a polypeptide derived from 6G described herein. In some embodiments, the antibody or polypeptide competitively inhibits binding of antibody 6G and/or antibody or polypeptide derived from 6G to Aβ₁₋₄₀ and Aβ₁₋₄₂. In some embodiments, the antibody is not antibody 2294 described in US 2004/0146512 and WO 04/032868. As described in WO2006118959, antibody 2294 binds to an epitope very similar to antibody 6G.

Methods of making antibodies and polypeptides are known in the art and described herein.

Competition assays can be used to determine whether two antibodies bind the same epitope by recognizing identical or sterically overlapping epitopes or one antibody competitively inhibits binding of another antibody to the antigen. These assays are known in the art. Typically, antigen is immobilized on a multi-well plate and the ability of unlabeled antibodies to block the binding of labeled antibodies is measured. Common labels for such competition assays are radioactive labels or enzyme labels.

### Polynucleotides, Vectors and Host Cells

The invention also provides isolated polynucleotides encoding the antibodies and polypeptides of the invention (including an antibody comprising the polypeptide sequences of the light chain and heavy chain variable regions shown in FIGS. 1 and 8), and vectors and host cells comprising the polynucleotide.

Accordingly, the invention provides polynucleotides (or compositions, including pharmaceutical compositions), comprising polynucleotides encoding any of the following: (a) antibody 9TL or 6G or their variants shown in Tables 3 and 8; (b) a fragment or a region of antibody 9TL or 6G or its variants shown in Tables 3 and 8; (c) a light chain of antibody 9TL or 6G or their variants shown in Tables 3 and 8; (d) a heavy chain of antibody 9TL or 6G or their variants shown in Tables 3 and 8; (e) one or more variable region(s) from a light chain and/or a heavy chain of antibody 9TL or 6G or their variants shown in Tables 3 and 8; (f) one or more CDR(s) (one, two, three, four, five or six CDRs) of antibody 9TL or 6G or their variants shown in Tables 3 and 8; (g) CDR H3 from the heavy chain of antibody 9TL or 6G; (h) CDR L3 from the light chain of antibody 9TL or 6G or their variants shown in Tables 3 and 8; (i) three CDRs from the light chain of antibody 9TL or 6G or their variants shown in Tables 3 and 8; (j) three CDRs from the heavy chain of antibody 9TL or 6G or their variants shown in Tables 3 and 8; (k) three CDRs from the light chain and three CDRs from the heavy chain, of antibody 6G or its variants shown in Tables 3 and 8; and (l) an antibody comprising any one of (b) through (k). In some embodiments, the polynucleotide comprises either or both of the polynucleotide(s) shown in SEQ ID NO:9, SEQ ID NO:10, SEQ ID NO: 34; and SEQ ID NO:35.

In another aspect, the invention provides polynucleotides encoding any of the antibodies (including antibody fragments) and polypeptides described herein, such as antibodies and polypeptides having impaired effector function. Polynucleotides can be made by procedures known in the art.

In another aspect, the invention provides compositions (such as a pharmaceutical compositions) comprising any of the polynucleotides of the invention. In some embodiments, the composition comprises an expression vector comprising a polynucleotide encoding the 9TL antibody as described herein. In other embodiment, the composition comprises an expression vector comprising a polynucleotide encoding any of the antibodies or polypeptides described herein. In still other embodiments, the composition comprises either or both of the polynucleotides shown in SEQ ID NO:9 and SEQ ID NO:10. Expression vectors, and administration of polynucleotide compositions are further described herein.

In another aspect, the invention provides a method of making any of the polynucleotides described herein.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably at least about 80% identity and most preferably at least about 90% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., (1989), for example.

Suitable cloning vectors may be constructed according to standard techniques, or may be selected from a large number of cloning vectors available in the art.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus).

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably 10 fold higher, even more preferably 20 fold higher than that of the corresponding endogenous antibody or protein of interest, if present, in the host cells. Screening the host cells for a specific binding to Aβ₁₋₄₀ is effected by an immunoassay or FACS. A cell overexpressing the antibody or protein of interest can be identified.

### Diagnostic Uses of 9TL or 6G Derived Antibodies and Anti-Aβ Antibodies Having Impaired Effector Function

Antibody 9TL or 6G which binds to C-terminus of one or more Aβ peptide may be used to identify or detect the presence or absence of the targeted Aβ in the eye. For simplicity, reference will be made generally to 9TL or 6G antibodies with the understanding that these methods apply to any of Aβ binding embodiments (such as polypeptides) described herein. Detection generally involves contacting a biological sample with an antibody described herein that binds to Aβ₁₋₄₀ and the formation of a complex between Aβ₁₋₄₀ and an antibody (e.g., 9TL) which binds specifically to Aβ₁₋₄₀. The formation of such a complex can be in vitro or in vivo. The term “detection” as used herein includes qualitative and/or quantitative detection (measuring levels) with or without reference to a control.

Any of a variety of known methods can be used for detection, including, but not limited to, immunoassay, using antibody that binds the polypeptide, e.g. by enzyme-linked immunosorbent assay (ELISA), radioimmunoassay (RIA) and the like; and functional assay for the encoded polypeptide, e.g. binding activity or enzymatic assay. In some embodiments, the antibody is detectably labeled. Other embodiments are known in the art and described herein.

Antibodies and polypeptides of the invention can be used in the detection, diagnosis and monitoring of an ophthalmic disease, condition, or disorder having altered or aberrant Aβ or βAPP expression. Thus, in some embodiments, the invention provides methods comprises contacting a specimen (sample) of an individual suspected of having altered or aberrant Aβ expression in the eye with an antibody or polypeptide of the invention and determining whether the level of the Aβ peptide differs from that of a control or comparison specimen. In other embodiments, the invention provides methods comprises contacting a specimen (sample) of an individual and determining level of the Aβ expression.

For diagnostic applications, the antibody may be labeled with a detectable moiety including but not limited to radioisotopes, fluorescent labels, and various enzyme-substrate labels. Methods of conjugating labels to an antibody are known in the art. In other embodiment of the invention, antibodies of the invention need not be labeled, and the presence thereof can be detected using a labeled antibody which binds to the antibodies of the invention.

The antibodies of the present invention may be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

The antibodies may also be used for in vivo diagnostic assays, such as in vivo imaging. Generally, the antibody is labeled with a radionuclide (such as ¹¹¹In, ⁹⁹Tc, ¹⁴C, ¹³¹I, ¹²⁵I, or ³H) so that the cells or tissue of interest can be localized using immunoscintiography.

The antibody may also be used as staining reagent in pathology, following techniques well known in the art.

Anti-Aβ antibodies having impaired effector function may be used for measuring retina function for diagnosis of subject at risk of or diagnosed with a retina-related degenerative ophthalmic disease, and assessing progress of any treatment and disease stage. In some embodiments, an anti-Aβ antibody having impaired effector function is administered to a subject, and level of Aβ in the plasma is measured, whereby an increase in plasma Aβ indicates presence and/or level of brain amyloid burden in the subject. These methods may be used to monitor effectiveness of the treatment and disease stage and to determine future dosing and frequency. Antibodies having impaired effector function may have a better safety profile and provide advantage for these diagnostic uses.

### Methods of Using Anti-AβAntibody for Therapeutic Purposes

The antibodies (including polypeptides), polynucleotides, and pharmaceutical compositions described herein can be used in methods for treating, preventing and inhibiting the development of an ophthalmic disease characterized by retina-related degeneration. The methods comprise administering to the subject an effective amount of an antibody that specifically binds to the protein or the protein deposit or a polynucleotide encoding the antibody, wherein the antibody has impaired effector function.

The antibodies (including polypeptides), polynucleotides, and pharmaceutical compositions described herein can be used in methods for treating, preventing and inhibiting the development of age-related macular degeneration, and other ophthalmic diseases such as age-related macular degeneration (both wet and dry), glaucoma, diabetic retinopathy (including diabetic macular edema), ruptures in Bruch's membrane, myopic degeneration, ocular tumors and other related retinal degenerative diseases. Such methods comprise administering the antibodies, polypeptides, or polynucleotides, or a pharmaceutical composition to the subject. In prophylactic applications, pharmaceutical compositions or medicaments are administered to a patient susceptible to, or otherwise at risk of, the ophthalmic disease in an amount sufficient to eliminate or reduce the risk, lessen the severity, or delay the outset of the disease, including biochemical, histological and/or behavioral symptoms of the disease, its complications and intermediate pathological phenotypes presenting during development of the disease. In therapeutic applications, compositions or medicaments are administered to a patient suspected of, or already suffering from such a disease in amount sufficient to cure, or at least partially arrest, the symptoms of the disease (biochemical, histological and/or behavioral), including its complications and intermediate pathological phenotypes in development of the disease.

Complications and intermediate pathological phenotypes of age-related macular degeneration include thick diffuse sub-retinal pigment ipthelium (RPE) deposits, lip-rich drusen-like deposits, thickening of Bruch's membrane, patchy regions of RPE atrophy and choroidal neovascularization.

The invention also provides methods of delaying development of retina degeneration and/or its symptoms in a subject comprising administering an effective dosage of a pharmaceutical composition comprising an antibody, a polypeptide, or a polynucleotide described herein to the subject.

This invention also provides methods of recovering or protecting retinal function in a subject comprising administering an effective dose of a pharmaceutical composition comprising an antibody, a polypeptide, or a polynucleotide described herein described herein to the subject.

This invention also provides methods of reducing amyloid plaques and/or reducing or slowing Aβ accumulation in the retina of a subject comprising administering an effective dose of a pharmaceutical composition comprising an antibody, a polypeptide, or a polynucleotide described herein to the subject.

This invention also provides methods of removing or clearing amyloid plaques and/or Aβ accumulation in the retina of a subject comprising administering an effective dose of a pharmaceutical composition comprising an antibody, a polypeptide, or a polynucleotide described herein to the subject. In some embodiments, the amyloid plaques are in the brain of the subject.

This invention also provides methods of reducing Aβ peptide in retinal tissue, inhibiting and/or reducing accumulation of Aβ peptide in retinal tissue, comprising administering an effective dose of a pharmaceutical composition comprising an antibody, a polypeptide, or a polynucleotide described herein to the subject. Aβ polypeptide may be in soluble, oligomeric, or deposited form. Oligomeric form of Aβ may be composed of 2-50 Aβ polypeptides, which can be a mixture of full length 1-40 and 1-42 peptides and/or any truncated version of the these peptides.

The invention also provides methods of improving or reversing retinal degeneration in ophthalmic diseases, comprising administering an effective dosage of a pharmaceutical composition comprising an antibody, a polypeptide, or a polynucleotide described herein to the subject.

The invention also provides methods for treating or preventing diseases associated with retinal degeneration, comprising administering to the subject an effective dosage of a pharmaceutical composition comprising an antibody that specifically binds to a beta-amyloid peptide or an aggregated form of a beta-amyloid peptide, wherein the antibody comprises an Fc region with a variation from a naturally occurring Fc region, wherein the variation results in impaired effector function, whereby the administration of the antibody causes less cerebral microhemorrhage than administration of an antibody without the variation.

The methods described herein (including prophylaxis or therapy) can be accomplished by a single direct injection at a single time point or multiple time points to a single or multiple sites. Administration can also be nearly simultaneous to multiple sites. Frequency of administration may be determined and adjusted over the course of therapy, and is based on accomplishing desired results. In some cases, sustained continuous release formulations of antibodies (including polypeptides), polynucleotides, and pharmaceutical compositions of the invention may be appropriate. Various formulations and devices for achieving sustained release are known in the art.

Patients, subjects, or individuals include mammals, such as human, bovine, equine, canine, feline, porcine, and ovine animals. The subject is preferably a human, and may or may not be afflicted with disease or presently show symptoms. The present methods can be administered prophylactically to the general population without the need for any assessment of the risk of the subject patient. The present methods are useful for individuals who do have a known genetic risk of age-related macular degeneration. Such individuals include those having relatives who have experienced the disease, and those whose risk is determined by analysis of genetic or biochemical markers.

The pharmaceutical composition that can be used in the above methods include, any of the antibodies, polypeptides, and/or polynucleotides described herein. In some embodiments, antibody is antibody 9TL or 6G or their variants shown in Tables 3 and 8. In some embodiments, the antibody is an antibody that specifically binds to an Aβ peptide and comprises a constant region having impaired effector function.

### Administration and Dosage

The antibody is preferably administered to the mammal in a carrier; preferably a pharmaceutically-acceptable carrier. Suitable carriers and their formulations are described in Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000. Typically, an appropriate amount of a pharmaceutically-acceptable salt is used in the formulation to render the formulation isotonic. Examples of the carrier include saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

The antibody can be administered to the mammal by injection (e.g., systemic, intravenous, intraperitoneal, subcutaneous, intramuscular, intraportal, intracerebral, intracerebralventricular, and intranasal), or by other methods, such as infusion, which ensure its delivery to the bloodstream in an effective form. The antibody may also be administered by isolated perfusion techniques, such as isolated tissue perfusion, to exert local therapeutic effects. In addition, the antibody of the present invention can be administered to the eye topically or in the form of an injection, such as an intravitreal injection, a sub-retinal injection or a bilateral injection. Further information on administration of the compounds to the eye can be found in Tolentino et al., Retina 24 (2004) 132-138; Reich et al., Molecular vision 9 (2003) 210-216.

Effective dosages and schedules for administering the antibody may be determined empirically, and making such determinations is within the skill in the art. Those skilled in the art will understand that the dosage of antibody that must be administered will vary depending on, for example, the mammal that will receive the antibody, the route of administration, the particular type of antibody used and other drugs being administered to the mammal. Guidance in selecting appropriate doses for antibody is found in the literature on therapeutic uses of antibodies, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389. A typical daily dosage of the antibody used alone might range from about 1 μg/kg to up to 100 mg/kg of body weight or more per day, depending on the factors mentioned above. Generally, any of the following doses may be used: a dose of at least about 50 mg/kg body weight; at least about 10 mg/kg body weight; at least about 3 mg/kg body weight; at least about 1 mg/kg body weight; at least about 750 μg/kg body weight; at least about 500 μg/kg body weight; at least about 250 μg/kg body weight; at least about 100 μg/kg body weight; at least about 50 μg/kg body weight; at least about 10 μg/kg body weight; at least about 1 μg/kg body weight, or more, is administered. Antibodies may be administered at lower doses or less frequent at the beginning of the treatment to avoid potential side effect, such as temporary cerebral amyloid angiopathy (CM).

In some embodiments, more than one antibody may be present. Such compositions may contain at least one, at least two, at least three, at least four, at least five different antibodies (including polypeptides) of the invention.

The antibody may also be administered to the mammal in combination with effective amounts of one or more other therapeutic agents. The antibody may be administered sequentially or concurrently with the one or more other therapeutic agents. The amounts of antibody and therapeutic agent depend, for example, on what type of drugs are used, the pathological condition being treated, and the scheduling and routes of administration but would generally be less than if each were used individually.

Following administration of antibody to the mammal, the mammal's physiological condition can be monitored in various ways well known to the skilled practitioner.

The above principles of administration and dosage can be adapted for polypeptides described herein.

A polynucleotide encoding an antibody or a polypeptide described herein may also be used for delivery and expression of the antibody or the polypeptide in a desired cell. It is apparent that an expression vector can be used to direct expression of the antibody. The expression vector can be administered systemically, intraperitoneally, intravenously, intramuscularly, subcutaneously, intrathecally, intraventricularly, orally, enterally, parenterally, intranasally, dermally, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions comprising a polynucleotide encoding an antibody of the invention can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740; 4,777,127; GB Patent No. 2,200,651; and EP 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA(see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0 524 968. Additional approaches are described in Philip, Mol. Cell. Biol. (1994) 14:2411, and in Woffendin, Proc. Natl. Acad. Sci. (1994) 91:1581.

### Kits

The invention also provides articles of manufacture and kits containing materials useful for treating the ophthalmic diseases described herein, such as age-related macular degeneration (both wet and dry), glaucoma, diabetic retinopathy (including diabetic macular edema), ruptures in Bruch's membrane, myopic degeneration, ocular tumors and other related retinal degenerative diseases. The article of manufacture comprises a container with a label. Suitable containers include, for example, bottles, vials, and test tubes. The containers may be formed from a variety of materials such as glass or plastic. The container holds a composition having an active agent which is effective for treating pathological ophthalmic conditions or for detecting or purifying Aβ or βAPP. The active agent in the composition is an antibody and preferably, comprises monoclonal antibodies specific for Aβ or βAPP. In some embodiments, the active agent comprises antibody 9TL or 6G any antibodies or polypeptides derived therefrom. In some embodiments, the active agent comprises an anti-Aβ antibody or polypeptide having impaired effector function. In some embodiments, the anti-Aβ antibody or polypeptide comprises a heavy chain constant region, wherein the constant region has impaired effector function. The label on the container indicates that the composition is used for treating pathological ophthalmic conditions such as AMD, and may also indicate directions for either in vivo or in vitro use, such as those described above.

The invention also provides kits comprising any of the antibodies (such as 9TL or 6G), polypeptides, polynucleotides described herein. In some embodiments, the kit of the invention comprises the container described above. In other embodiments, the kit of the invention comprises the container described above and a second container comprising a buffer. It may further include other materials desirable from a commercial and user standpoint, including other buffers, diluents, filters, needles, syringes, and package inserts with instructions for performing any methods described herein (such as methods for treating AMD, and methods for inhibiting or reducing accumulation of Aβ peptide in the brain). In kits to be used for detecting or purifying Aβ or βAPP, the antibody is typically labeled with a detectable marker, such as, for example, a radioisotope, fluorescent compound, bioluminescent compound, a chemiluminescent compound, metal chelator or enzyme.

In some embodiments, the invention provides compositions (described herein) for use in any of the methods described herein, whether in the context of use as a medicament and/or use for manufacture of a medicament.

The following examples are provided to illustrate, but not to limit, the invention.

### EXAMPLES

### Example 1

### Binding Affinity Determination of Antibody 9TL and its Variants

### A. General Methods

The following general methods were used in this example.

Expression Vector Used in Clone Characterization

Expression of the Fab fragment of the antibodies was under control of an IPTG inducible lacZ promotor similar to that described in Barbas (2001) Phage display: a laboratory manual, Cold Spring Harbor, N.Y., Cold Spring Harbor Laboratory Press pg 2.10. Vector pComb3X), however, modifications included addition and expression of the following additional domains: the human Kappa light chain constant domain and the CHI constant domain of IgG2a human immunoglobulin, Ig gamma-2 chain C region, protein accession number P01859; Immunoglobulin kappa light chain (homosapiens), protein accession number CAA09181.

Small Scale Fab Preparation

Small scale expression of Fabs in 96 wells plates was carried out as follows. Starting from E. coli transformed with a Fab library, colonies were picked to inoculate both a master plate (agar LB+Ampicillin (50 μg/ml)+2% Glucose) and a working plate (2 ml/well, 96 well/plate containing 1.5 mL of LB+Ampicillin (50 μg/ml)+2% Glucose). Both plates were grown at 30° C. for 8-12 hours. The master plate was stored at 4° C. and the cells from the working plate were pelleted at 5000 rpm and resuspended with 1 mL of LB+Ampicillin (50 μg/ml)+1 mM IPTG to induce expression of Fabs. Cells were harvested by centrifugation after 5 h expression time at 30° C., then resuspended in 500 μL of buffer HBS-P (10 mM HEPES buffer pH 7.4, 150 mM NaCl, 0.005% P20). Lysis of HBS-P resuspended cells was attained by one cycle of freezing (−80° C.) then thawing at 37° C. Cell lysates were centrifuged at 5000 rpm for 30 min to separate cell debris from supernatants containing Fabs. The supernatants were then injected into the BIAcore plasmon resonance apparatus to obtain affinity information for each Fab. Clones expressing Fabs were rescued from the master plate to sequence the DNA and for large scale Fab production and detailed characterization as described below.

Large Scale Fab Preparation

To obtain detailed kinetic parameters, Fabs were expressed and purified from large cultures. Erlenmeyer flasks containing 200 mL of LB+Ampicillin (50 μg/ml)+2% Glucose were inoculated with 5 mL of over night culture from a selected Fab-expressing E. coli clone. Clones were incubated at 30° C. until an OD_{550 nm} of 1.0 was attained and then induced by replacing the media for 200 ml, of LB+Ampicillin (50 μg/ml)+1 mM IPTG. After 5 h expression time at 30° C., cells were pelleted by centrifugation, then resuspended in 10 mL PBS (pH 8). Lysis of the cells was obtained by two cycles of freeze/thaw (at −80° C. and 37° C., respectively). Supernatant of the cell lysates were loaded onto Ni-NTA superflow sepharose (Qiagen, Valencia. CA) columns equilibrated with PBS, pH 8, then washed with 5 column volumes of PBS, pH 8. Individual Fabs eluted in different fractions with PBS (pH 8)+300 mM Imidazol. Fractions containing Fabs were pooled and dialized in PBS, then quantified by ELISA prior to affinity characterization.

Full Antibody Preparation

For expression of full antibodies, heavy and light chain variable regions were cloned in mammalian expression vectors and transfected using lipofectamine into HEK 293 cells for transient expression. Antibodies were purified using protein A using standard methods. Vector pDb.9TL.hFc2a is an expression vector comprising the heavy chain of the 9TL antibody, and is suitable for transient or stable expression of the heavy chain. Vector pDb.9TL.hFc2a has nucleotide sequences corresponding to the following regions: the murine cytomegalovirus promoter region (nucleotides 1-612); a synthetic intron (nucleotides 619-1507); the DHFR coding region (nucleotides 707-1267); human growth hormone signal peptide (nucleotides 1525-1602); heavy chain variable region of 9TL (nucleotides 1603-1951); human heavy chain IgG2a constant region containing the following mutations: A330P331 to S330S331 (amino acid numbering with reference to the wildtype IgG2a sequence; see Eur. J. Immunol. (1999) 29:2613-2624); SV40 late polyadenylation signal (nucleotides 2960-3203); SV40 enhancer region (nucleotides 3204-3449); phage f1 region (nucleotides 3537-4992) and beta lactamase (AmpR) coding region (nucleotides 4429-5286). Vector pDb.9TL.hFc2a was deposited at the ATCC on Jul. 20, 2004, and was assigned ATCC Accession No. PTA-6124.

Vector pEb.9TL.hK is an expression vector comprising the light chain of the 9TL antibody, and is suitable for transient expression of the light chain. Vector pEb.9TL.hK has nucleotide sequences corresponding to the following regions: the murine cytomegalovirus promoter region (nucleotides 1-612); human EF-1 intron (nucleotides 619-1142); human growth hormone signal peptide (nucleotides 1173-1150); antibody 9TL light chain variable region (nucleotides 1251-1593); human kappa chain constant region (nucleotides 1594-1914); SV40 late polyadenylation signal (nucleotides 1932-2175); SV40 enhancer region (nucleotides 2176-2421); phage f1 region (nucleotides 2509-2964) and beta lactamase (AmpR) coding region (nucleotides 3401-4258). Vector pEb.9TL.hK was deposited at the ATCC on Jul. 20, 2004, and was assigned ATCC Accession No. PTA-6125.

Biacore Assay

Affinities of 9TL monoclonal antibody were determined using the BIAcore3000™ surface plasmon resonance (SPR) system (BIAcore, INC, Piscaway N.J.). One way of determining the affinity was immobilizing of 9TL on CM5 chip and measuring binding kinetics of Aβ₁₋₄₀ peptide to the antibody. CM5 chips were activated with N-ethyl-N′-(3-dimethylaminopropyl)-carbodiimide hydrochloride (EDC) and N-hydroxysuccinimide (NHS) according to the supplier's instructions. Antibody 9TL or its variants was diluted into 10 mM sodium acetate pH 4.0 or 5.0 and injected over the activated chip at a concentration of 0.005 mg/mL. Using variable flow time across the individual chip channels, a range of antibody density was achieved: 1000-2000 or 2000-3000 response units (RU). The chip was blocked with ethanolamine. Regeneration studies showed that a solution containing 2 volumes of PIERCE elution buffer and 1 volumes of 4 M NaCl effectively removed the bound Aβ₁₋₄₀ peptide while keeping the activity of 9TL on the chip for over 200 injections. HBS-EP buffer (0.01M HEPES, pH 7.4, 0.15 M NaCl, 3 mM EDTA, 0.005% Surfactant P20) was used as running buffer for all the BIAcore assays. Serial dilutions (0.1-10× estimated K_{D}) of purified Aβ₁₋₄₀ synthetic peptide samples were injected for 1 min at 100 μL/min and dissociation times of 10 min were allowed. Kinetic association rates (kₒₙ) and dissociation rates (k_{off}) were obtained simultaneously by fitting the data to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (K_{D}) values were calculated as k_{off}/kₒₙ.

Alternatively, affinity was determined by immobilizing Aβ₁₋₄₀ peptide on SA chip and measuring binding kinetics of 9TL Fab and Fab of 9TL variants to the immobilized Aβ₁₋₄₀ peptide. Affinities of 9TL Fab fragment and its variants Fab fragments were determined by Surface Plasmon Resonance (SPR) system (BIAcore 3000™, BIAcore, Inc., Piscaway, N.J.). SA chips (streptavidin) were used according to the supplier's instructions. Biotinylated Aβ peptide 1-40 was diluted into HBS-EP (10 mM HEPES pH 7.4, 150 mM NaCl, 3 mM EDTA, 0.005% P20) and injected over the chip at a concentration of 0.005 mg/mL. Using variable flow time across the individual chip channels, two ranges of antigen density were achieved: 10-200 response units (RU) for detailed kinetic studies and 500-600 RU for concentration studies and screening. Regeneration studies showed that 100 mM phosphoric acid (may also be followed by a solution containing 2 volumes of 50 mM NaOH and 1 volume of 70% ethanol) effectively removed the bound Fab while keeping the activity of Aβ peptide on the chip for over 200 injections. HBS-EP buffer was used as running buffer for all the BIAcore assays. Serial dilutions (0.1-10× estimated K_{D}) of purified Fab samples were injected for 2 min at 100 μL/min and dissociation times of 10 min were allowed. The concentrations of the Fab proteins were determined by ELISA and/or SDS-PAGE electrophoresis using a standard Fab of known concentration (determined by amino acid analysis). Kinetic association rates (kₒₙ) and dissociation rates (k_{off}) were obtained simultaneously by fitting the data to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (K_{D}) values were calculated as k_{off}/kₒₙ.

### B. Binding Affinity of Antibody 9TL and its Variants to Aβ₁₋₄₀

The amino acid sequences of the heavy chain and light chain variable regions of antibody 9TL is shown in FIG. 1. The binding affinity of 9TL antibody to Aβ₁₋₄₀ determined using both methods of Biacore described above is shown in Table 2 below.

TABLE 2
Binding affinity of antibody 9TL and Fab fragment

| | kₒₙ (1/Ms) | K_{off} (1/s) | K_{D} (nM) |
|---|---|---|---|
| 9TL mAb on CM5 chip, Aβ₁₋₄₀ | 4.25 × 10⁵ | 3.89 × 10⁻⁴ | 0.9 |
| flowed onto it | | | |
| Aβ₁₋₄₀ on SA chip, 9TL Fab | 3.18 × 10⁵ | 3.59 × 10⁻⁴ | 1.13 |
| flowed onto it | | | |

The amino acid sequence of the variants of 9TL is shown in Table 3 below. All amino acid substitutions of the variants shown in Table 3 are described relative to the sequence of 9TL. The binding affinity of Fab fragment of 9TL variants are also shown in Table 3. K_{D} and other kinetic parameters were determined by BIAcore analysis described above with Aβ₁₋₄₀ immobilized on SA chip.

TABLE 3
Amino acid sequences and kinetic data for antibody 9TL variants.

| | | | | | | | kₒₙ | k_{off} | K_{D} |
|---|---|---|---|---|---|---|---|---|---|
| Clone | H1 (1) | H2 | H3 | L1 | L2 | L3 | (Ms⁻¹) (2) | (s⁻¹) | (nM) (3) |
| 9TL | | | | | | | 3.18 × 10⁵ | 3.59 × 10⁻⁴ | 1.13 |
| 22-T/I | | | | | | L102I | 3.18 × 10⁵ | 4.60 × 10⁻⁴ | 1.45 |
| C6 new | | | | | | L102T | 3.56 × 10⁵ | 9.20 × 10⁻⁴ | 2.58 |
| W1 | | | | Y31A, | | L102T | 3.18 × 10⁵ | 9.00 × 10⁻³ | 28.30 |
| | | | | A34S | | | | | |
| W8 | | | | Y31H, | | L102T | 3.18 × 10⁵ | 3.80 × 10⁻³ | 11.95 |
| | | | | A34S, | | | | | |
| | | | | K35A | | | | | |
| W5 | | | | Y31H, | | L102T | 3.18 × 10⁵ | 4.00 × 10⁻³ | 12.58 |
| | | | | K35A | | | | | |
| M1 | | | | | | L94M | 3.18 × 10⁵ | 8.60 × 10⁻⁴ | 2.70 |
| M2 | | | | | | L94N | 3.18 × 10⁵ | 1.10 × 10⁻³ | 3.46 |
| M3 | | | | | | L94C | 3.18 × 10⁵ | 1.30 × 10⁻³ | 4.09 |
| M4 | | | | | | L94F | 3.18 × 10⁵ | 9.95 × 10⁻⁴ | 3.13 |
| M5 | | | | | | L94V | 3.18 × 10⁵ | 1.65 × 10⁻³ | 5.19 |
| M6 | | | | | | L94K | 3.18 × 10⁵ | 4.10 × 10⁻³ | 12.89 |
| M7 | | | | | | L94S | 3.18 × 10⁵ | 6.00 × 10⁻³ | 18.87 |
| M8 | | | | | | L94Q | 3.18 × 10⁵ | 6.80 × 10⁻³ | 21.38 |
| M9 | | | | | | L94G | 3.18 × 10⁵ | 7.80 × 10⁻³ | 24.53 |
| M10 | | | | | | L94S | 3.18 × 10⁵ | 8.30 × 10⁻³ | 26.10 |
| M11 | | | | | | G96S | 3.18 × 10⁵ | 2.00 × 10⁻³ | 6.29 |
| M12 | | | | | | G96T | 3.18 × 10⁵ | 3.30 × 10⁻³ | 10.38 |
| M13 | | | | | | T97S | 3.18 × 10⁵ | 3.90 × 10⁻⁴ | 1.23 |
| M14 | | | | | | H98L | 3.18 × 10⁵ | 1.60 × 10⁻³ | 5.03 |
| M15 | | | | | | Y99P | 3.18 × 10⁵ | 6.70 × 10⁻⁴ | 2.11 |
| M16 | | | | | | Y99A | 3.18 × 10⁵ | 7.00 × 10⁻⁴ | 2.20 |
| M17 | | | | | | Y99W | 3.18 × 10⁵ | 1.00 × 10⁻³ | 3.14 |
| M18 | | | | | | Y99Q | 3.18 × 10⁵ | 1.50 × 10⁻³ | 4.72 |
| M19 | | | | | | Y99M | 3.18 × 10⁵ | 1.70 × 10⁻³ | 5.35 |
| M20 | | | | | | Y99S | 3.18 × 10⁵ | 2.00 × 10⁻³ | 6.29 |
| M21 | | | | | | Y99E | 3.18 × 10⁵ | 5.00 × 10⁻³ | 15.72 |
| M22 | | | | | | V101L | 3.18 × 10⁵ | 4.00 × 10⁻³ | 12.58 |
| M23 | | | | | | V101K | 3.18 × 10⁵ | 5.00 × 10⁻³ | 15.72 |
| M24 | | | | | | V101H | 3.18 × 10⁵ | 6.00 × 10⁻³ | 18.87 |
| M25 | | | | | | V101T | 3.18 × 10⁵ | 8.00 × 10⁻³ | 25.16 |
| M26 | | | | | | V101A | 3.18 × 10⁵ | 9.00 × 10⁻³ | 28.30 |
| M27 | | | | | | V101E | 3.18 × 10⁵ | 1.20 × 10⁻² | 37.74 |
| M28 | | | | | | V101M | 3.18 × 10⁵ | 1.40 × 10⁻² | 44.03 |
| M29 | | | | | | L102S | 3.18 × 10⁵ | 7.60 × 10⁻⁴ | 2.39 |
| M30 | | | | | | L102V | 3.18 × 10⁵ | 6.80 × 10⁻⁴ | 2.14 |
| M31 | | | L99V | | | | 3.18 × 10⁵ | 1.00 × 10⁻² | 31.45 |
| M32 | | | L99I | | | | 3.18 × 10⁵ | 2.00 × 10⁻² | 62.89 |
| M33 | | | Y100W | | | | 3.18 × 10⁵ | 6.30 × 10⁻⁴ | 1.98 |
| M34 | | | S101T | | | | 3.18 × 10⁵ | 8.00 × 10⁻⁴ | 2.52 |
| M35 | | | S101G | | | | 3.18 × 10⁵ | 9.00 × 10⁻³ | 28.30 |
| M36 | | | L102R | | | | 3.18 × 10⁵ | 9.00 × 10⁻⁴ | 2.83 |
| M37 | | | L102A | | | | 3.18 × 10⁵ | 9.20 × 10⁻⁴ | 2.89 |
| M38 | | | L102V | | | | 3.18 × 10⁵ | 1.50 × 10⁻³ | 4.72 |
| M39 | | | L102S | | | | 3.18 × 10⁵ | 2.30 × 10⁻³ | 7.23 |
| M40 | | | L102T | | | | 3.18 × 10⁵ | 4.50 × 10⁻³ | 14.15 |
| M41 | | | L102Q | | | | 3.18 × 10⁵ | 1.00 × 10⁻² | 31.45 |
| M42 | | | L102E | | | | 3.18 × 10⁵ | 1.50 × 10⁻² | 47.17 |
| M43 | | | V104I | | | | 3.18 × 10⁵ | 3.00 × 10⁻⁴ | 0.94 |
| M44 | | | V104T | | | | 3.18 × 10⁵ | 3.00 × 10⁻³ | 9.43 |
| M45 | | | V104P | | | | 3.18 × 10⁵ | 1.50 × 10⁻² | 47.17 |
| M46 | | | V104C | | | | 3.18 × 10⁵ | 2.00 × 10⁻² | 62.89 |
| M47 | | | V104Q | | | | 3.18 × 10⁵ | 2.00 × 10⁻² | 62.89 |
| M48 | | | V104S | | | | 3.18 × 10⁵ | 2.60 × 10⁻² | 81.76 |
| M49 | | | V104N | | | | 3.18 × 10⁵ | 2.60 × 10⁻² | 81.76 |
| M50 | | | V104F | | | | 3.18 × 10⁵ | 2.70 × 10⁻² | 84.91 |
| M51 | | | Y105H | | | | 3.18 × 10⁵ | 8.60 × 10⁻⁴ | 2.70 |
| M52 | | | Y105F | | | | 3.18 × 10⁵ | 1.30 × 10⁻³ | 4.09 |
| M53 | | | Y105W | | | | 3.18 × 10⁵ | 1.30 × 10⁻³ | 4.09 |
| M54 | | | Y105S | | | | 3.18 × 10⁵ | 2.40 × 10⁻³ | 7.55 |
| M55 | | | Y105I | | | | 3.18 × 10⁵ | 3.00 × 10⁻³ | 9.43 |
| M56 | | | Y105V | | | | 3.18 × 10⁵ | 3.50 × 10⁻³ | 11.01 |
| M57 | | | Y105A | | | | 3.18 × 10⁵ | 3.90 × 10⁻³ | 12.26 |

(1) = All CDRs are extended CDRs including both kabat and Chothia CDRs. Amino acid residues are numbered sequentially.
(2) = underlined kₒₙ were experimentally determined. Others were estimated to be the same as 9TL.
(3) = K_{D} values were calculated as K_{D} = k_{off}/kₒₙ.

### Example 2

### Characterization of Epitope on A(1-40 Peptide that Antibody 9TL Binds

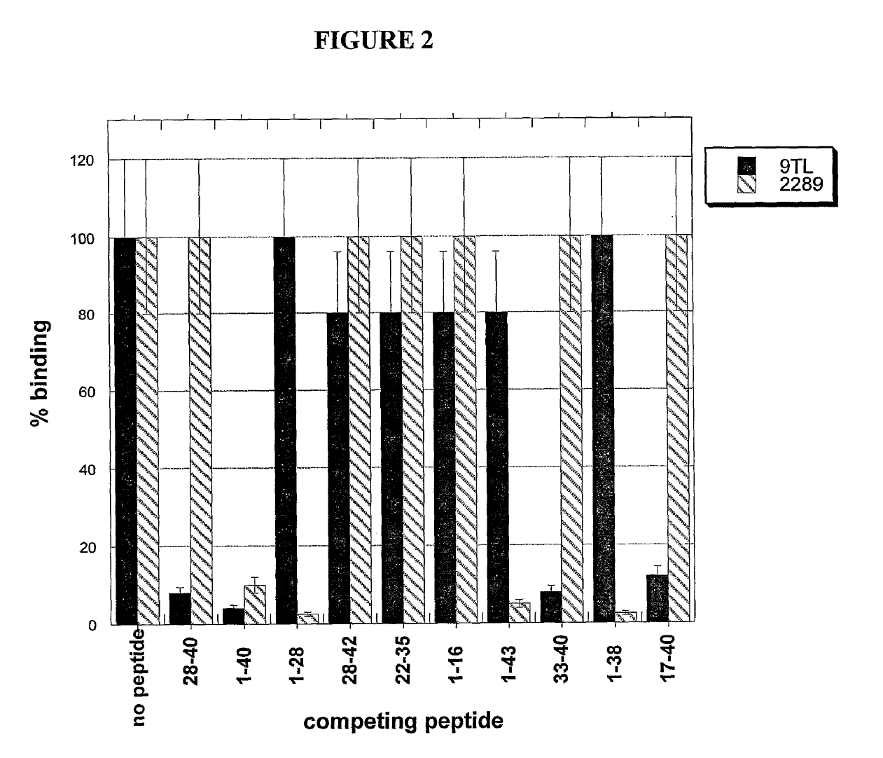

To determine the epitope on Aβ polypeptide that is recognized by antibody 9TL, Surface Plasmon Resonance (SPR, Biacore 3000) binding analysis was used. Aβ₁₋₄₀ polypeptide coupled to biotin (Global Peptide Services, CO) was immobilized on a streptavidin-coated chip (SA chip). The binding of Aβ antibodies Fab fragments (at 50 nM) to the immobilized Aβ₁₋₄₀ in the absence or presence of different soluble fragments of the Aβ peptide (at 10 pM, from American Peptide Company Inc., CA). Amino acid sequences of Aβ₁₋₄₀, Aβ₁₋₄₂, and Aβ₁₋₄₃ are shown in below in Table 4. The Aβ peptides which displaced binding of antibody 9TL Fab fragment to Aβ₁₋₄₀ were Aβ₂₈₄₀, Aβ₁₋₄₀, Aβ₃₃₋₄₀, and Aβ₁₇₋₄₀, respectively (FIG. 2). Thus, antibody 9TL binds to a C-terminal peptide (33-40) of Aβ₁₋₄₀. As shown in FIG. 2, the Aβ₁₋₂₈, Aβ₂₈₋₄₂, Aβ₂₂₋₃₅, Aβ₁₋₁₆, Aβ₁₋₄₃, and Aβ₁₋₃₈ peptide did not inhibit the binding of antibody 9TL Fab fragment, suggesting that antibody 9TL binds to the C-terminus of Aβ₁₋₄₀ peptide.

In addition, Aβ₂₈₋₄₂ and Aβ₁₋₄₃ peptide did not inhibit binding of antibody 9TL to Aβ₁₋₄₀ although they could readily inhibit Aβ₁₋₄₀ binding to control antibody (antibody 2289, this antibody is described in U.S. Appl. Pub. No. 2004/0146512 and WO04/032868) which bind to 16-28 of Aβ₁₋₄₀. These results show that antibody 9TL preferentially binds to Aβ₁₋₄₀, but not to Aβ₁₋₄₂ and Aβ₁₋₄₃.

TABLE 4
Amino acid sequences of beta amyloid peptides

| | |
|---|---|
| 1-40 (WT) | DAEFRHDSGYEVHHQKLVFFAEDVGSNKGAIIGLMVGGVV |
| | (SEQ ID NO:15) |
| 1-42 (WT) | DAEFRHDSGYEVHHQKLVFFAEDVGSNKGAIIGLMVGGVV |
| | IA |
| | (SEQ ID NO:16) |
| 1-43 (WT) | DAEFRHDSGYEVHHQKLVFFAEDVGSNKGAIIGLMVGGVV |
| | IAT |
| | (SEQ ID NO:17) |

### Example 3

### Generation of Monoclonal Antibody 2H6 and Deglycosylated 2H6

A. Generation and Characterization of Monoclonal Antibody 2H6

Mice were immunized with 25-100 μg of a peptide (amino acid 28-40 of Aβ₁₋₄₀) conjugated to KLH in adjuvant (50 μl per footpad, 100 μl total per mouse) at about 16 consecutive week intervals as described in Geerligs H J et al., 1989, J. Immunol. Methods 124:95-102; Kenney J S et al., 1989, J. Immunol. Methods 121:157-166; and Wicher K et al., 1989, Int. Arch. Allergy Appl. Immunol. 89:128-135. Mice were first immunized with 50 μg of the peptide in CFA (complete Freud's adjuvant). After 21 days, mice were secondly immunized with 25 μg of the peptide in IFA (incomplete Freud's adjuvant). Twenty three days later after the second immunization, third immunization was performed with 25 μg of the peptide in IFA. Ten days later, antibody titers were tested using ELISA. Fourth immunization was performed with 25 μg of the peptide in IFA 34 days after the third immunization. Final booster was performed with 100 μg soluble peptide 32 days after the fourth immunization.

Splenocytes were obtained from the immunized mouse and fused with NSO myeloma cells at a ratio of 10:1, with polyethylene glycol 1500. The hybrids were plated out into 96-well plates in DMEM containing 20% horse serum and 2-oxaloacetate/pyruvate/insulin (Sigma), and hypoxanthine/aminopterin/thymidine selection was begun. On day 8, 100 μl of DMEM containing 20% horse serum was added to all the wells. Supernatants of the hybrids were screened by using antibody capture immunoassay. Determination of antibody class was done with class-specific second antibodies. A panel of monoclonal antibody-producing cell lines was selected for characterization. One cell line selected produces as antibody designated 2H6. This antibody was determined to have IgG2b heavy chain.

The affinity of antibody 2H6 to Aβ₁₋₄₀ was determined. Monoclonal antibody 2H6 was purified from supernatants of hybridoma cultures using protein A affinity chromatography. The supernatants was equilibrated to pH 8. The supernatants were then loaded to the protein A column MabSelect (Amersham Biosciences # 17-5199-02) equilibrated with PBS to pH 8. The column was washed with 5 column volumes of PBS, pH 8. The antibody was eluted with 50 mM citrate-phosphate buffer, pH 3. The eluted antibody was neutralized with 1M Phosphate Buffer, pH 8. The purified antibody was dialyzed with PBS. The antibody concentration was determined by SDS-PAGE, using a murine mAb standard curve.

2H6 Fabs were prepared by papain proteolysis of the 2H6 full antibody using Immunopure Fab kit (pierce # 44885) and purified by flow through protein A chromatography following manufacturer instructions. Concentration was determined by SDS-PAGE and A280 using 1OD=0.6 mg/ml.

Affinities of 2H6 monoclonal antibody were determined using the BIAcore3000™ surface plasmon resonance (SPR) system (BIAcore, INC, Piscaway N.J.). One way of determining the affinity was immobilizing 2H6 antibody on CM5 chip and measuring binding kinetics of Aβ₁₋₄₀ peptide to the antibody. CM5 chips were activated with N-ethyl-N′-(3-dimethylaminopropyl)-carbodiimide hydrochloride (EDC) and N-hydroxysuccinimide (NHS) according to the supplier's instructions. 2H6 monoclonal antibody was diluted into 10 mM sodium acetate pH 4.0 or 5.0 and injected over the activated chip at a concentration of 0.005 mg/mL. Using variable flow time across the individual chip channels, a range of antibody density was achieved: 1000-2000 or 2000-3000 response units (RU). The chip was blocked with ethanolamine. Regeneration studies showed that a mixture of Pierce elution buffer (Product No. 21004, Pierce Biotechnology, Rockford, Ill.) and 4 M NaCl (2:1) effectively removed the bound Aβ₁₋₄₀ peptide while keeping the activity of 2H6 antibody on the chip for over 200 injections. HBS-EP buffer (0.01M HEPES, pH 7.4, 0.15 M NaCl, 3 mM EDTA, 0.005% Surfactant P20) was used as running buffer for all the BIAcore assays. Serial dilutions (0.1-10× estimated K_{D}) of purified Aβ₁₋₄₀ synthetic peptide samples were injected for 1 min at 100 μL/min and dissociation times of 10 min were allowed. Kinetic association rates (kₒₙ) and dissociation rates (k_{off}) were obtained simultaneously by fitting the data to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (K_{D}) values were calculated as k_{off}/kₒₙ.

Alternatively, affinity was determined by immobilizing Aβ₁₋₄₀ peptide on SA chip and measuring binding kinetics of 2H6 Fab to the immobilized Aβ₁₄₀ peptide. Affinities of 2H6 Fab fragment was determined by Surface Plasmon Resonance (SPR) system (BIAcore 3000, BIAcore, Inc., Piscaway, N.J.). SA chips (streptavidin) were used according to the supplier's instructions. Biotinylated Aβ peptide 1-40 (SEQ ID NO:15) was diluted into HBS-EP (10 mM HEPES pH 7.4, 150 mM NaCl, 3 mM EDTA, 0.005% P20) and injected over the chip at a concentration of 0.005 mg/mL. Using variable flow time across the individual chip channels, two ranges of antigen density were achieved: 10-200 response units (RU) for detailed kinetic studies and 500-600 RU for concentration studies. Regeneration studies showed that a mixture of Pierce elution buffer and 4 M NaCl (2:1) effectively removed the bound Fab while keeping the activity of Aβ peptide on the chip for over 200 injections. HBS-EP buffer was used as running buffer for all the BIAcore assays. Serial dilutions (0.1-10× estimated K_{D}) of purified Fab samples were injected for 2 min at 100 μL/min and dissociation times of 10 min were allowed. The concentrations of the Fab proteins were determined by ELISA and/or SDS-PAGE electrophoresis using a standard Fab of known concentration (determined by amino acid analysis). Kinetic association rates (kₒₙ) and dissociation rates (k_{off}) were obtained simultaneously by fitting the data to a 1:1 Langmuir binding model (Karlsson, R. Roos, H. Fagerstam, L. Petersson, B. (1994). Methods Enzymology 6. 99-110) using the BIAevaluation program. Equilibrium dissociation constant (K_{D}) values were calculated as k_{off}/kₒₙ. The affinity of 2H6 antibody determined using both methods described above is shown in Table 5 below.

Affinity for murine antibody 2286, which binds to a peptide of amino acid 28-40 of Aβ₁₋₄₀, was tested as described above. Antibody 2286 is described in U.S. application Ser. No. 10/683,815 and PCT/US03/32080.

TABLE 5
Binding affinity of antibody 2H6 and 2286

| | kₒₙ (1/Ms) | K_{off} (1/s) | K_{D} (nM) |
|---|---|---|---|
| 2H6 mAb on CM5 chip, Aβ₁₋₄₀ | 4.67 × 10⁵ | 3.9 × 10⁻³ | 9 |
| flowed on | | | |
| Aβ₁₋₄₀0 on SA chip, 2H6 Fab | 6.3 × 10⁵ | 3.0 × 10⁻³ | 4.7 |
| flowed on | | | |
| 2286 mAb on CM5 chip, Aβ₁₋₄₀ | 1.56 × 10⁵ | 0.0419 | 269 |
| flowed on | | | |
| Aβ₁₋₄₀ on SA chip, 2286 Fab | 1.8 × 10⁵ | 0.044 | 245 |
| flowed on | | | |

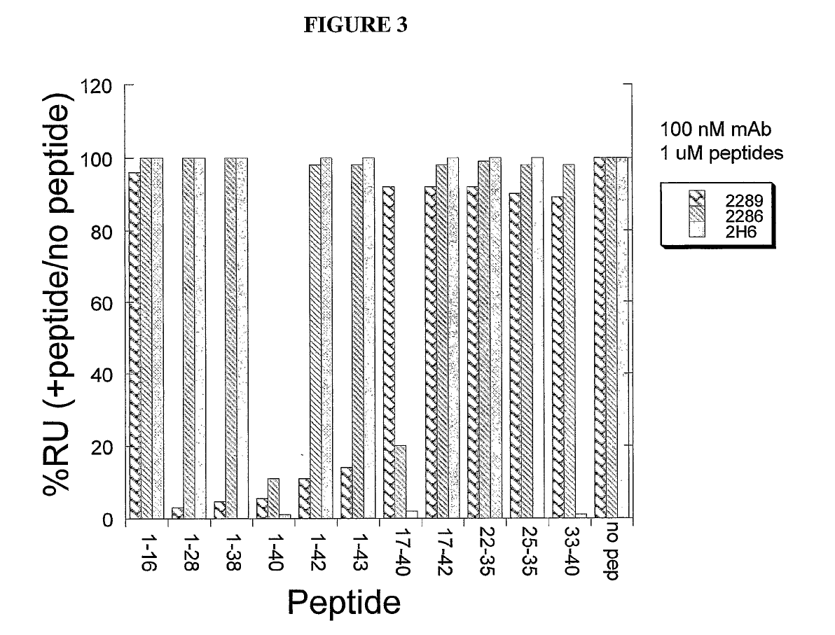

To determine the epitope on Aβ polypeptide recognized by antibody 2H6, Surface Plasmon Resonance (SPR, Biacore 3000) binding analysis was used. Aβ₁₋₄₀ polypeptide (SEQ ID NO:15) coupled to biotin (Global Peptide Services, CO) was immobilized on a streptavidin-coated chip (SA chip). The binding of Aβ antibodies (at 100 nM) to the immobilized Aβ₁₋₄₀ in the absence or presence of different soluble fragments of the Aβ peptide (at 16 pM, from American Peptide Company Inc., CA). The Aβ peptides which displaced binding of antibody 2H6 to Aβ₁₋₄₀ were Aβ₁₇₄₀, Aβ₃₃₄₀, and Aβ₁₋₄₀, respectively (FIG. 3). Thus, antibody 2H6 binds to a C-terminal peptide (33-40) of Aβ₁₋₄₀. However, this C-terminal peptide (33-40) of Aβ₁₋₄₀ did not displace binding of antibody 2286 to Aβ₁₋₄₀ at the concentration tested. As shown in FIG. 3, the Aβ₁₋₃₈ peptide did not inhibit the binding of antibody 2H6 or antibody 2286 to Aβ₁₋₄₀, suggesting that, similar to antibody 2286, the epitope that antibody 2H6 binds includes amino acids 39 and/or 40 of the Aβ₁₋₄₀ peptide (FIG. 3).

In addition, Aβ₁₋₄₂ and Aβ₁₋₄₃ peptide did not inhibit binding of antibody 2H6 to Aβ₁₋₄₀ although they could readily inhibit Aβ₁₋₄₀ binding to control antibody (antibody 2289, this antibody is described in U.S. application Ser. No. 10/683,815 and PCT/US03/32080) which bind to 16-28 of Aβ₁₋₄₀ (FIG. 3). These results show that antibody 2H6 preferentially binds to Aβ₁₋₄₀, but not to Aβ₁₋₄₂ and Aβ₁₋₄₃.

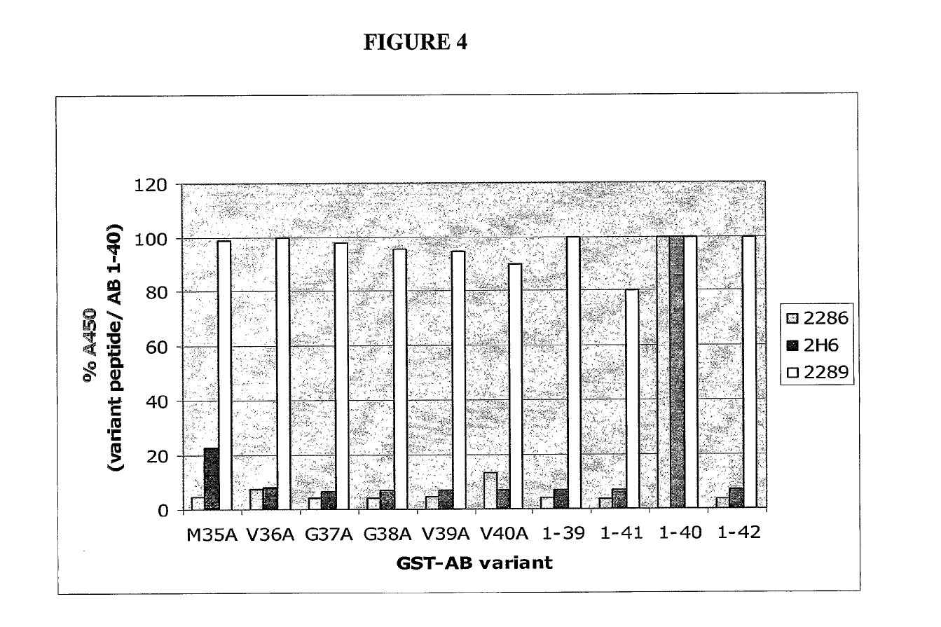

To further assess the involvement of discrete amino acid residues of the β-amyloid peptide that antibody 2H6 binds, different Aβ₁₋₄₀ variants, in which each of the last 6 amino acids (Aβ₁₋₄₀ amino acid residues 35-40) was individually replaced by an alanine (alanine scanning mutagenesis), were generated by site directed mutagenesis. These Aβ₁₋₄₀ variants (sequences shown in Table 6) were expressed in E. coli as Glutathione-S-Transferase (GST) fusion proteins (Amersham Pharmacia Biotech, Piscataway, N.J. USA) followed by affinity purification on a Glutathione-Agarose beads (Sigma-Aldrich Corp., St. Louis, Mo., USA). As control, Wild-type (WT) A□₁₋₄₀ as well as Aβ₁₋₄₁, Aβ₁₋₄₂, and Aβ₁₋₃₉ were also expressed as GST fusion proteins. Aβ₁₋₄₀, Aβ₁₋₄₁, Aβ₁₋₄₂, Aβ₁₋₃₉, as well as the six different variants (M35A(1-40), V36A(1-40), G37A(1-40), G38A(1-40), V39A(1-40), V40A(1-40) shown in Table 6) were then immobilized (100 μl of 0.025 μg/μl of GST-peptide per well) onto ELISA assay plates and incubated with either of mAb 2286, 2289, and 2H6 in serial dilution from 0.3 nM down (data using 0.3 nM mAb are shown in FIG. 4). After 10 consecutive washes, assay plates were incubated with 100 μl of 0.03 μg/ml per well of Biotin-conjugated Goat-anti-Mouse (H+L) antibody (Vector Laboratories, vector #BA-9200, Burilingame Calif., USA) followed by 100 μl of 0.025 μg/ml per well of HRP-conjugated Streptavidin (Amersham Biosciences Corp., #RPN4401V, NJ, USA). The absorbance of the plate was read at 450 nm.

TABLE 6

Amino acid sequences of beta amyloid peptides
and variants

| | | |
|---|---|---|
| 1-40 (WT) | DAEFRHDSGYEVHHQKLVFFAEDV | (SEQ ID NO:15) |
| | GSNKGAIIGLMVGGVV | |
| 1-42 (WT) | DAEFRHDSGYEVHHQKLVFFAEDV | (SEQ ID NO:16) |
| | GSNKGAIIGLMVGGVVIA | |
| 1-43 (WT) | DAEFRHDSGYEVHHQKLVFFAEDV | (SEQ ID NO:17) |
| | GSNKGAIIGLMVGGVVIAT | |
| 1-41 (WT) | DAEFRHDSGYEVHHQKLVFFAEDV | (SEQ ID NO:18) |
| | GSNKGAIIGLMVGGVVI | |
| 1-39 (WT) | DAEFRHDSGYEVHHQKLVFFAEDV | (SEQ ID NO:19) |
| | GSNKGAIIGLMVGGV | |
| M35A(1-40) | DAEFRHDSGYEVHHQKLVFFAEDV | (SEQ ID NO:20) |
| | GSNKGAIIGLAVGGVV | |
| V36A(1-40) | DAEFRHDSGYEVHHQKLVFFAEDV | (SEQ ID NO:21) |
| | GSNKGAIIGLMAGGVV | |
| G37A(1-40) | DAEFRHDSGYEVHHQKLVFFAEDV | (SEQ ID NO:22) |
| | GSNKGAIIGLMVAGVV | |
| G38A(1-40) | DAEFRHDSGYEVHHQKLVFFAEDV | (SEQ ID NO:23) |
| | GSNKGAIIGLMVGAVV | |
| V39A(1-40) | DAEFRHDSGYEVHHQKLVFFAEDV | (SEQ ID NO:24) |
| | GSNKGAIIGLMVGGAV | |
| V40A(1-40) | DAEFRHDSGYEVHHQKLVFFAEDV | (SEQ ID NO:25) |
| | GSNKGAIIGLMVGGVA | |

As shown in FIG. 4, Mab 2289 which was directed to amino acid 16 to 28 of A [symbol] recognized all variants with the same intensity and served as internal positive control of protein concentration and protein integrity on the plate. Antibody 2H6 did not recognize Aβ₁₋₄₁, Aβ₁₋₃₉, or Aβ₁₋₄₂ as shown in FIG. 4. Aβ₁₋₄₀ variants V40A, V39A, G38A, G37A, V36A, and M35A showed reduced binding to antibody 2H6, demonstrating that antibody 2H6 epitope extended for at least 6 amino acids at the C terminal end of Aβ₁₋₄₀. Mutations of V and G to A are very conservative and are not likely to produce important conformational changes in proteins, therefore, the large effect of these mutations to antibody 2H6 binding might be due to the ability of the antibody to differentiate between the mentioned amino acids in the context of Aβ and these data demonstrated a very high degree of specificity for this antibody.

To determine whether 2H6 and 9TL compete for binding to Aβ₁₋₄₀, competition experiments were performed using Biacore assay. Antibody 2H6, 9TL and 2289 were immobilized on different channels of a CM5 chip. CM5 chip channels were activated with N-ethyl-N′-(3-dimethylaminopropyl)-carbodiimide hydrochloride (EDC) and N-hydroxysuccinimide (NHS) according to the supplier's instructions. Antibody 2H6, 9TL, and 2289 were each diluted into 10 mM sodium acetate pH 4.0 and injected over an activated chip at a concentration of 0.005 mg/mL. Antibody density was 1625 response units (RU) for 2H6; 4000 RU for 9TL; and 2200 RU for 2289. Each channel was blocked with ethanolamine. Aβ₁₋₄₀ peptide (150 uM) was flowed onto the chip for 2 min. Then antibody 2H6 (to be tested for competition of binding) at 0.6 uM was flowed onto the chip for 1 min. HBS-EP buffer (0.01M HEPES, pH 7.4, 0.15 M NaCl, 3 mM EDTA, 0.005% Surfactant P20) was used as running buffer for all the BIAcore assays. After measuring binding of Aβ₁₋₄₀, all channels of the chip were regenerated by washing twice with a mixture of Pierce elution buffer (Product No. 21004, Pierce Biotechnology, Rockford, Ill.) and 4 M NaCl (2:1) for 6 sec. Competition binding was then performed for antibody 9TL, and then antibody 2289. Competition between 9TL and 2H6 for binding to Aβ₁₋₄₀ was observed, but no competition was observed between 9TL and 2289 or between 2H6 and 2289. Observations of competition between the antibody immobilized and the same antibody flowed onto the chip served as the positive control.

### B. Antibody 2H6 Does Not Bind to APP

To determine whether 2H6 binds to amyloid precursor proteins (APP), binding of 2H6 to cells transfected with wildtype APP was determined. 293 cells were transfected with a cDNA encoding wild type human amyloid precursor protein. Forty eight hours after the transfection, cells were incubated on ice for 45 minutes with monoclonal antibodies anti-Aβ₁₋₁₆, anti-Aβ₁₆₋₂₈, or 2H6 (5 ug/ml in DMEM with 10% FCS). The cells were then washed three times in PBS for 5 minutes, fixed with 4% PFA. The cells were washed three times again in PBS, and antibody binding was detected with secondary Cy3-conjugated goat anti-mouse antibody (dilution of 1:500) from Jackson Immunoresearch under fluorescence microscope. Anti-Aβ₁₋₁₆ and anti-Aβ₁₆₋₂₈ antibodies, which recognize N-terminal or central epitopes in Aβ, both showed significant binding to APP precursor proteins expressed on cells. In contrast, 2H6 did not bind to APP expressing cells.

### C. Generation of Deglycosylated Antibody 2H6

To generate deglycosylated antibody 2H6, purified antibody 2H6 was incubated at 37° C. for 7 days with peptide-N-glycosidase F (Prozyme, 0.05 Upper mg of antibody) in 20 mM Tris-HCl pH 8.0. Completeness of deglycosylation was verified by MALDI-TOF-MS and protein gel electrophoresis. Deglycosylated antibodies were purified by Protein A chromatography and endotoxin was removed by Q-Sepharose. The binding affinity to Aβ₁₋₄₀ of the deglycosylated 2H6 was tested using Biacore assay described above, and the binding affinity of the deglycosylated 2H6 to Aβ₁₋₄₀ was found to be identical to the intact antibody 2H6.

### Example 4

### Effect of Deglycosylated Antibody 2H6 (2H6-D) in Protection and Recovery of Retinal Function in Animal Model of Age-Related Macular Degeneration

Initial studies (Malek, G. et al., PNAS 102: 11900-5 (2005)) demonstrated that the combination of three risk factors: (1) apolipoprotein isoform E4 (APOE4) genotype, (2) advanced age (over 65 weeks) and (3) high fat and cholesterol rich (HF-C) diet produced an animal model that closely approximated the clinical features of human AMD. Aged APOE4 mice developed pathological changes similar to the morphologic hallmarks observed in both dry and wet human AMD, including retinal pigment epithelial (RPE)-pigmentary changes, thick diffuse sub-RPE deposits, lipid-rich drusen-like deposits, thickening of Bruch's membrane, patchy regions of RPE atrophy overlying photoreceptor degeneration and choroidal neovascularization (CNV). These changes were not detected in any of the control human APOE3 expressing mice regardless of dietary regimen, nor were any pathologies detected in young APOE4 animals. Functional deficits were identified from full field scotopic electroretinograms. Affected animals had a significant reduction in a- and b-wave amplitudes compared to controls. Importantly, these histopathological and functional changes required the presence of all three risk factors. This animal model of spontaneously occurring CNV is the first to incorporate physiologically-relevant risk factors of human disease.

### A. Experimental Protocol

Administration of antibodies. Targeted-replacement mice expressing human ApoE4 aged (over 65 weeks) were used for the experiments. The AMD-like phenotype present in these mice has been previously disclosed (Malek, G. et al., PNAS 102: 11900-5 (2005)). For the eight weeks treatment study, ApoE4-transgenic mice, aged 65 weeks or more, were assigned to one of the four groups. The first group (E4-ND) was continuously maintained on a normal diet (n=2). The second group (E4-HFC-R1) was fed a high fat, cholesterol enriched (HF-C) diet 8 weeks (n=2). The third group (E4-HFC-R1) was fed an HF-C diet for 8 weeks and received weekly intraperitoneal injections of Rinat 1 (3 mg/kg) (n=5). The fourth group (E4-HFC-R2) was fed a HF-C diet for 8 weeks and received weekly intraperitoneal injections of Rinat 2 (3 mg/kg) (n=5). After completion of the study, the groups were unmasked: Rinat 1 was PBS vehicle and Rinat 2 was deglycosylated anti-Aβ antibody 2H6 (mouse monoclonal anti-human Aβ₂₈₋₄₀ IgG2b ‘2H6-D’ as described in Example 3).

In vivo assessments. Fundus examination was performed at week 0, while fundus and fluorescein angiograms were performed at week 8. Photographs were taken, using a fundus camera (TRC-50EX Retina Camera). Images were captured using the TOPCON IMAGEnet™ system. Fluorescein dye (10% fluorescein sodium, approximately 0.1 mL/kg) was injected via vascular access ports. Photographs were taken at several timepoints following dye injection, to include the arterial phase, early arteriovenous phase and several late arteriovenous phases in order to evaluate neovascularization and to monitor leakage of fluorescein associated with CNV lesions. Interpretation and analysis of the fluorescein angiograms was independently conducted by an ophthalmologist. Total plasma cholesterol levels in whole blood were collected from the mice (fasted for 5 hours) before and after administration of the 8-week HF-C diet.

Fluorescein Angiography (FA). One animal, an E4-HFC-R2, showed possible leakage in late frames of the angiogram. The animal died following FA but before electroretinagrams and no tissue could be recovered.

Electroretinogram Recordings. During the ninth week, electroretinogram (ERG) recordings were obtained from animals, dark adapted for at least 12 hours. Each animal was anesthetized with a ketamine/xylazine cocktail, pupils were dilated and after the animal stabilized on a 37° C. warming pad, ERG tracings were recorded using a silver wire test electrode placed in contact with the eye along with a drop of 2.5% hydroxypropyl methylcellulose. Mice were placed in a photopic stimulator chamber where the animal was exposed to flashes of light (max intensity of 1000 cd-s/m² attenuate in 1 log steps, starting from 0.0005). The a-wave amplitude was measured from baseline to the a-wave trough, and the b-wave amplitude was measured from the a-wave trough to the b-wave peak.

Histological analysis. On the day of sacrifice, mice were weighed, overdosed with Avertin (0.2 □l/10 gm body weight), and then intracardially perfused with 20 mL saline. Brains were rapidly removed, and the left half of the brain was immersion fixed for 16 h in freshly prepared 10% formalin for histopathology. Thirty micron vibratome sections were pretreated with 10% MeOH, 1×PBS, and 2% H2O2, washed with PBS, incubated in 88% formic acid for 1 minute for antigen retrieval and blocked with 5% normal goat serum (NGS) and PBS. Sections were incubated overnight with a 1:1000 dilution of biotinylated 4G8 primary antibody (Signet 4G8 monoclonal mouse human IgG2b against amino acid residues 17-24 of B-amyloid) in 1% NGS/PBS and visualized using ABC Vectastain Kit (Vector Labs) as described by manufacturer.

### B. Results

Recovery/Protection of Retinal Function by Administration of Deglycosylated Antibody.

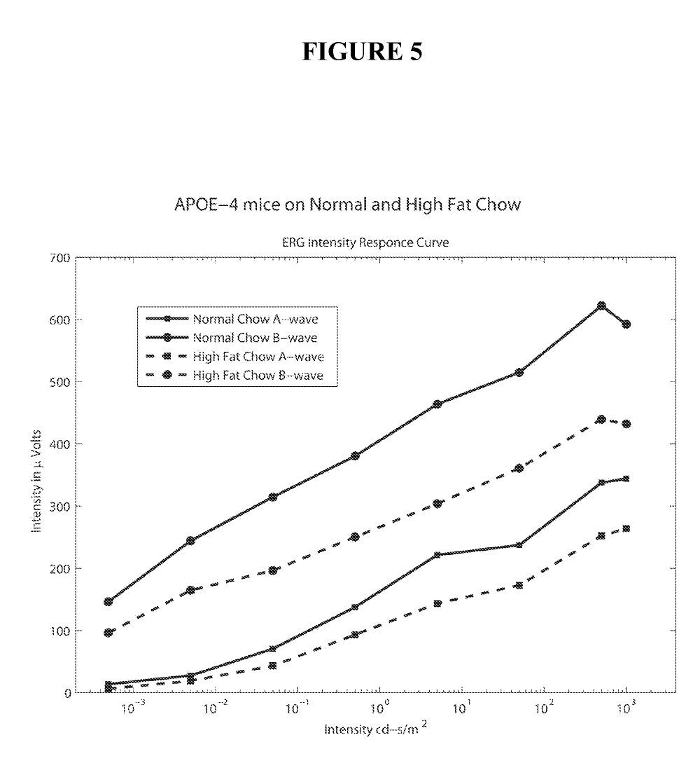

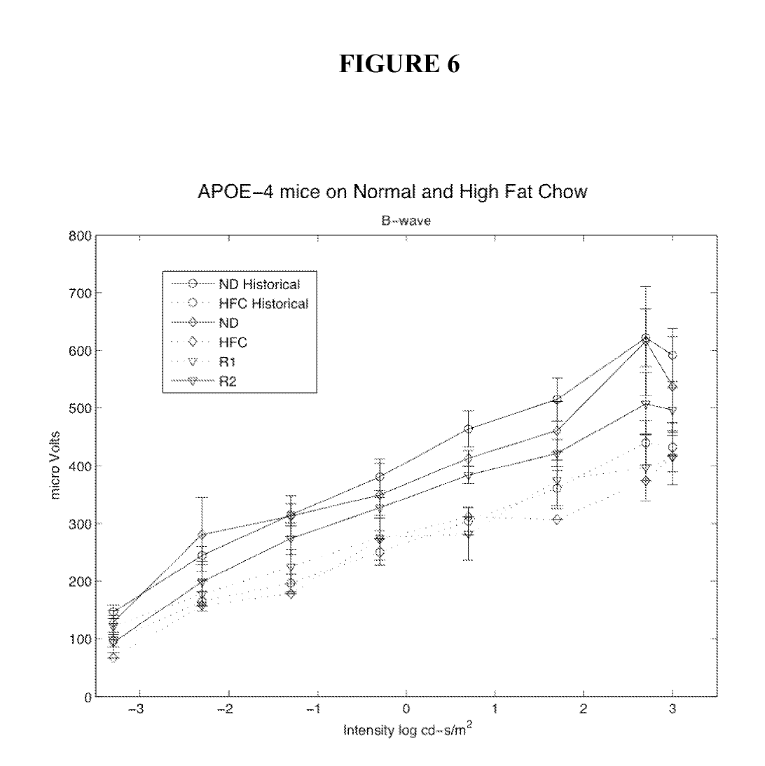

As shown in FIG. 5, there is a statistically significant reduction in the a- and b-wave amplitudes in ApoE4 mice fed the high fat and cholesterol rich diet (E4-HFC) versus mice on the normal diet (E4-ND) (a-wave p=0.0106, b-wave p=0.008). ERGs obtained from the injected animals were compared to our baseline set of ERGs. There was no significant difference in the a-wave amplitudes of either injected group (E4-HFC-R1 and E4-HFC-R2) compared to E4-HFC (not shown). In contrast, the b-wave amplitudes show a striking recovery and/or protection of retinal function in the E4-HFC-R2 group (FIG. 6).

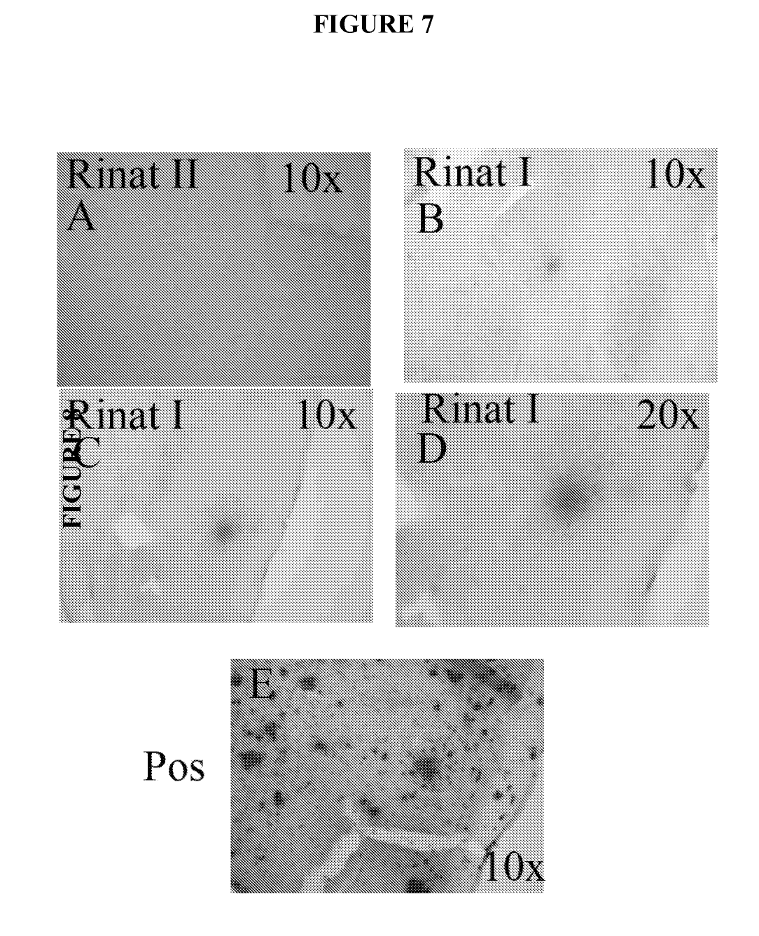

Reduction of Aβ deposits in APOE4Mice on HF-C Diet Injected with Anti-AβAntibody 2H6-D. As illustrated in FIG. 7, total Aβ immunostaining in the E4-HFC mice was reduced after 8 weeks immunotherapy with antibody 2H6-D as compared to the control vehicle group.

### C. Conclusion

The above data demonstrate 1) recovery/protection of retinal function as demonstrated by ERGs of mice injected with Anti-AβAntibody 2H6-D and 2) reduction of amyloid deposition when treated with antibody 2H6-D in mouse brain as compared to the untreated AMD mouse group.

### Example 5

### Binding Affinity Determination of Antibody 6G and its Variants

A. General methods

The following general methods were used in this example and other examples.

### Expression Vector Used in Clone Characterization

Expression of the Fab fragment of the antibodies was under control of an IPTG inducible lacZ promotor similar to that described in Barbas (2001) Phage display: a laboratory manual, Cold Spring Harbor, N.Y., Cold Spring Harbor Laboratory Press pg 2.10. Vector pComb3X), however, modifications included addition and expression of the following additional domains: the human Kappa light chain constant domain and the CHI constant domain of IgG2a human immunoglobulin, Ig gamma-2 chain C region, protein accession number P01859; Immunoglobulin kappa light chain (homosapiens), protein accession number CAA09181.

Small Scale Fab Preparation

Small scale expression of Fabs in 96 wells plates was carried out as follows. Starting from E. coli transformed with a Fab library, colonies were picked to inoculate both a master plate (agar LB+Ampicillin (50 μg/ml)+2% Glucose) and a working plate (2 ml/well, 96 well/plate containing 1.5 mL of LB+Ampicillin (50 μg/ml)+2% Glucose). Both plates were grown at 30° C. for 8-12 hours. The master plate was stored at 4° C. and the cells from the working plate were pelleted at 5000 rpm and resuspended with 1 mL of LB+Ampicillin (50 μg/ml)+1 mM IPTG to induce expression of Fabs. Cells were harvested by centrifugation after 5 h expression time at 30° C., then resuspended in 500 μL of buffer HBS-EP (100 mM HEPES buffer pH 7.4, 150 mM NaCl, 0.005% P20). Lysis of HBS-EP resuspended cells was attained by one cycle of freezing (−80° C.) then thawing at 37° C. Cell lysates were centrifuged at 5000 rpm for 30 min to separate cell debris from supernatants containing Fabs. The supernatants were then injected into the BIAcore plasmon resonance apparatus to obtain affinity information for each Fab. Clones expressing Fabs were rescued from the master plate to sequence the DNA and for large scale Fab production and detailed characterization as described below.

Large Scale Fab Preparation

To obtain detailed kinetic parameters, Fabs were expressed and purified from large cultures. Erlenmeyer flasks containing 200 mL of LB+Ampicillin (50 μg/ml)+2% Glucose were inoculated with 5 mL of over night culture from a selected Fab-expressing E. coli clone. Clones were incubated at 30° C. until an OD₅₅₀ nm of 1.0 was attained and then induced by replacing the media for 200 ml, of LB+Ampicillin (50 μg/ml)+1 mM IPTG. After 5 h expression time at 30° C., cells were pelleted by centrifugation, then resuspended in 10 mL PBS (pH 8). Lysis of the cells was obtained by two cycles of freeze/thaw (at −80° C. and 37° C., respectively). Supernatant of the cell lysates were loaded onto Ni-NTA superflow sepharose (Qiagen, Valencia. CA) columns equilibrated with PBS, pH 8, then washed with 5 column volumes of PBS, pH 8. Individual Fabs eluted in different fractions with PBS (pH 8)+300 mM Imidazol. Fractions containing Fabs were pooled and dialized in PBS, then quantified by ELISA prior to affinity characterization.

Full Antibody Preparation

For expression of full antibodies, heavy and light chain variable regions were cloned in mammalian expression vectors and transfected using lipofectamine into HEK 293 cells for transient expression. Antibodies were purified using protein A using standard methods.

Vector pDb.6G.hFc2a is an expression vector comprising the heavy chain of the 6G antibody, and is suitable for transient or stable expression of the heavy chain. Vector pDb.6G.hFc2a has nucleotide sequences corresponding to the following regions: the murine cytomegalovirus promoter region (nucleotides 1-612); a synthetic intron (nucleotides 619-1507); the DHFR coding region (nucleotides 707-1267); human growth hormone signal peptide (nucleotides 1525-1602); heavy chain variable region of 6G; human heavy chain IgG2a constant region containing the following mutations: A330P331 to S330S331 (amino acid numbering with reference to the wildtype IgG2a sequence; see Eur. J. Immunol. (1999) 29:2613-2624); SV40 late polyadenylation signal; SV40 enhancer region; phage f1 region and beta lactamase (AmpR) coding region.

Vector pEb.6G.hK is an expression vector comprising the light chain of the 6G antibody, and is suitable for transient expression of the light chain. Vector pEb.6G.hK has nucleotide sequences corresponding to the following regions: the murine cytomegalovirus promoter region (nucleotides 1-612); human EF-1 intron (nucleotides 619-1142); human growth hormone signal peptide (nucleotides 1173-1150); antibody 6G light chain variable region; human kappa chain constant region; SV40 late polyadenylation signal; SV40 enhancer region; phage f1 region and beta lactamase (AmpR) coding region.

### Biacore Assay

Affinities of 6G monoclonal antibody were determined using the BIAcore3000™ surface plasmon resonance (SPR) system (BIAcore, INC, Piscaway N.J.) using methods described in Example 1 above.

### ELISA Assay

ELISA was used for measuring binding of antibody 6G and variants to nonbiotinylated Aβ peptides. NUNC maxisorp plates were coated with 2.5 ug/ml of Aβ peptides in PBS pH 7.4 for more than 1 hour at 4° C. Plates were blocked with 1% BSA in PBS buffer pH 7.4. Primary antibody (from cell supernatants, serum containing anti-Aβ antibody, or purified full antibody or Fabs at desired dilution) was incubated with the immobilized Aβ peptides for 1 h at room temperature. After washing, the plates were incubated with secondary antibody, a HRP conjugated goat anti-human kappa chain antibody (MP Biomedicals, 55233) at 1:5000 dilution. After washing, bound secondary antibody was measured by adding TMB substrate (KPL, 50-76-02, 50-65-02). The HRP reaction was stopped by adding 1M phosphoric acid and absorbance at 450 nm was measured.

ELISA was used for measuring binding of antibody 6G and variants to biotinylated Aβ peptides. NUNC maxisorp plates were coated with 6 ug/ml of streptavidin (Pierce, 21122) in PBS pH 7.4 for more than 1 h at 4° C. Plates were blocked with 1% BSA in PBS buffer pH 7.4. After washing, biotinylated Aβ peptides in PBS pH 7.4 were incubated 1 hour at room temperature. Primary antibody (from cell supernatants, serum containing anti-Aβ antibody, or purified full antibody or Fabs at desired dilution) was incubated with the immobilized Aβ peptides for 1 h at room temperature. After washing, plates were incubated with secondary antibody, a HRP conjugated goat anti-human kappa chain antibody (MP Biomedicals, 55233) at 1:5000 dilution. After washing, bound secondary antibody was measured by adding TMB substrate (KPL, 50-76-02, 50-65-02). HRP reaction was stopped by adding 1M phosphoric acid and absorbance at 450 nm was measured.

B. Binding Affinity of Antibody 6G and Variants to Aβ₁₋₄₀, Aβ₁₋₄₂, and other Aβ Peptides

The amino acid sequences of the heavy chain and light chain variable regions of antibody 6G is shown in FIG. 8. The binding affinity of 6G antibody to Aβ₁₋₄₀, Aβ₁₋₄₂, and Aβ₂₂₋₃₇ determined using Biacore described above is shown in Table 7 below.

TABLE 7
Binding affinity of antibody 6G Fab fragment

| | | | K_{D} |
|---|---|---|---|
| | kₒₙ (1/Ms) | k_{off} (1/s) | (nM) |
| Biotinylated Aβ₁₋₄₀ immobilized on | 3.0 × 10⁵ | 7.0 × 10⁻⁴ | 2 |
| streptavidin chip, 6G Fab flowed onto it | | | |
| Biotinylated Aβ₁₋₄₂ immobilized on | 1.8 × 10⁴ | 1.6 × 10⁻³ | 80 |
| streptavidin chip, 6G Fab flowed onto it | | | |
| Biotinylated Aβ₂₂₋₃₇ immobilized on | 3.6 × 10⁵ | 3.9 × 10⁻³ | 11 |
| streptavidin chip, 6G Fab flowed onto it | | | |

The amino acid sequence of the variants of 6G is shown in Table 8 below. All amino acid substitutions of the variants shown in Table 8 are described relative to the sequence of 6G. The relative binding of 6G variants are also shown in Table 8. Binding was determined by ELISA described above with nonbiotinylated Aβ₁₋₄₀ or Aβ₁₋₄₂ immobilized on the surface of an ELISA plate.

TABLE 8
Amino acid sequences and binding data for antibody 6G variants.

| | |
|---|---|
| 6G Heavy chain mutant variants binding data by ELISA | A450 ELISA |

| | | | |
|---|---|---|---|
| clone number | mutations | Aβ₁₋₄₀ | Aβ₁₋₄₂ |

| | | | | | | | | |
|---|---|---|---|---|---|---|---|---|
| 6G | F99 | D100 | N101 | Y102 | D103 | R104 | 2.55 | 0.95 |
| 1A | Y | | | | | | 1.60 | 0.26 |
| 1B | M | | | | | | 0.37 | 0.22 |
| 1G | L | | | | | | 0.51 | 0.21 |
| 2G | P | | | | | | 0.30 | 0.50 |
| 3E | C | | | | | | 0.26 | 0.40 |
| 4G | | S | | | | | 1.41 | 0.30 |
| 5D | | N | | | | | 1.52 | 0.39 |
| 6A | | T | | | | | 0.86 | 0.31 |
| 7B | | | S | | | | 0.44 | 0.27 |
| 7D | | | C | | | | 0.23 | 0.31 |
| 8H | | | H | | | | 0.21 | 0.19 |
| 9E | | | R | | | | 0.22 | 0.26 |
| 10A | | | | F | | | 1.85 | 0.34 |
| 10E | | | | L | | | 0.41 | 0.24 |
| 10G | | | | I | | | 0.63 | 0.22 |
| 11D | | | | M | | | 0.29 | 0.24 |
| 2F | | | | | P | | 1.89 | 0.38 |
| 3A | | | | | A | | 1.16 | 0.28 |
| 3B | | | | | R | | 1.43 | 0.43 |
| 3C | | | | | G | | 2.30 | 0.76 |
| 4A | | | | | | G | 2.17 | 0.40 |
| 4B | | | | | | F | 2.48 | 0.71 |
| 4D | | | | | | Q | 2.45 | 1.00 |
| 6F | | | | | | S | 2.28 | 0.62 |

| 6G Heavy chain mutant variants binding data by ELISA | | | | | | | | | | A450 ELISA | |
|---|---|---|---|---|---|---|---|---|---|---|---|
| clone number | mutations | | | | | | | | | Aβ₁₋₄₀ | Aβ₁₋₄₂ |
| 6G | Q93 | Q94 | S95 | K96 | E97 | F98 | P99 | W100 | S101 | 2.49 | 0.61 |
| 2H | K | | | | | | | | | 0.07 | 0.13 |
| 3A | P | | | | | | | | | 0.08 | 0.13 |
| 4F | S | | | | | | | | | 2.00 | 0.30 |
| 5B | | G | | | | | | | | 0.09 | 0.14 |
| 7E | | R | | | | | | | | 0.09 | 0.18 |
| 7F | | K | | | | | | | | 0.12 | 0.19 |
| 10E | | | L | | | | | | | 0.08 | 0.12 |
| 1A | | | | N | | | | | | 2.02 | 0.32 |
| 10 | | | | F | | | | | | 0.05 | 0.05 |
| 4A | | | | A | | | | | | 2.09 | 0.28 |
| 4G | | | | F | | | | | | 1.07 | 0.28 |
| 5H | | | | | R | | | | | 2.60 | 0.85 |
| 6C | | | | | G | | | | | 0.05 | 0.05 |
| 6D | | | | | T | | | | | 2.41 | 1.34 |
| 6E | | | | | P | | | | | 0.12 | 0.20 |
| 8G | | | | | V | | | | | 2.60 | 0.90 |

### Example 6

### Characterization of Epitope on Aβ Peptide that Antibody 6G Binds

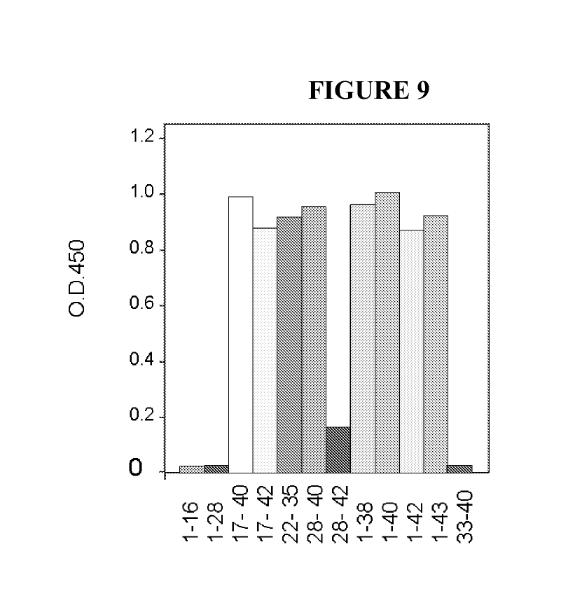

To determine the epitope on Aβ peptide that is recognized by antibody 6G, ELISA binding analysis was used. Various Aβ peptides (Global Peptide Services, CO) was immobilized on a ELISA plate. The binding of 6G full antibody (at 20 nM) to the immobilized Aβ was determined by ELISA as described above. Amino acid sequences of Aβ₁₋₄₀, Aβ₁₋₄₂, and Aβ₁₋₄₃ are shown in Table 9 below. As shown in FIG. 9, antibody 6G binds to Aβ peptides 17-40, 17-42, 22-35, 28-40, 1-38, 1-40, 1-42, 1-43, and 28-42; but binding to 28-42 is much weaker than the other Aβ peptides. Antibody 6G did not bind to Aβ peptide 1-16, 1-28 and 33-40. Thus, antibody 6G binds to the C-terminus of various truncated Aβ peptide, for example, 22-35, 1-38, 1-40, 1-42, and 1-43.

Table 9 below shows binding affinity comparison of 6G to Aβ₁₋₄₀ to other Aβ peptide as measured by k_{off} (1/s) using Biacore assay. Antibody 6G binds to Aβ₁₋₄₀ with highest affinity as compared to other peptides, 5 with significantly lower affinity to truncated Aβ₁₋₄₀ (such 1-36, 1-37, 1-38, and 1-39), Aβ₁₋₄₂ and Aβ₁₋₄₃. This indicates that the side chain or backbone of amino acid 40 (Valine) of Aβ is involved in binding of 6G to Aβ₁₋₄₀; and binding is significantly reduced (for example from about 10 to about 50-250 fold loss of affinity) in absence of this amino acid. Binding with lower affinity to carboxy-terminal amidated Aβ₁₋₄₀ indicates that binding of 6G to Aβ₁₋₄₀ involves but is not dependent on the free C-terminus of Aβ₁₋₄₀. Lower affinity binding to Aβ₁₋₄₂ and Aβ₁₋₄₃ may be due to conformational differences between monomer form of Aβ₁₋₄₀ and Aβ₁₋₄₂ or Aβ₁₋₄₃. It has been shown that monomer of Aβ₁₋₄₂ has a conformation different from Aβ₁₋₄₀ monomer in solution. See, the monomer structure coordinate for Aβ₁₋₄₂ shown in Protein Data Bank (pdb files) with accession no. 1 IYT; and the monomer structure coordinate for Aβ₁₋₄₀ shown in Protein Data Bank (pdb files) with accession nos. 1 BA6 and 1 BA4.

TABLE 9

| Aβ peptide | | K_{off} Aβ peptide/k_{off} Aβ₁₋₄₀ |
|---|---|---|
| fragment | k_{off} (1/s) | (fold loss of affinity) |
| 1-28 | — | |
| 1-43 | Very low binding | |
| 22-35 | 0.0285 | 215.9 |
| 1-36 | 0.0205 | 155.3 |
| 1-37 | 0.0149 | 112.8 |
| 1-38 | 9.3 × 10⁻³ | 70.4 |
| 1-39 | 7.92 × 10⁻³ | 60.0 |
| 17-42 | 0.0465 | 352.2 |
| 1-42 | 1.9 × 10⁻³ | 14.4 |
| 28-42 | 3.37 × 10⁻³ | 25.5 |
| 28-40-NH2# | 3.62 × 10⁻³ | 27.4 |
| 28-40 | 6.4 × 10⁻⁴ | 4.8 |
| 17-40 | 2.15 × 10⁻⁴ | 1.6 |
| 1-40 | 1.32 × 10⁻⁴ | 1 |

Peptide flowed as analyte onto a CM5 chip with 6G monoclonal antibody (ligand) immobilized by amine chemistry
#peptide with carboxy-terminal amidated

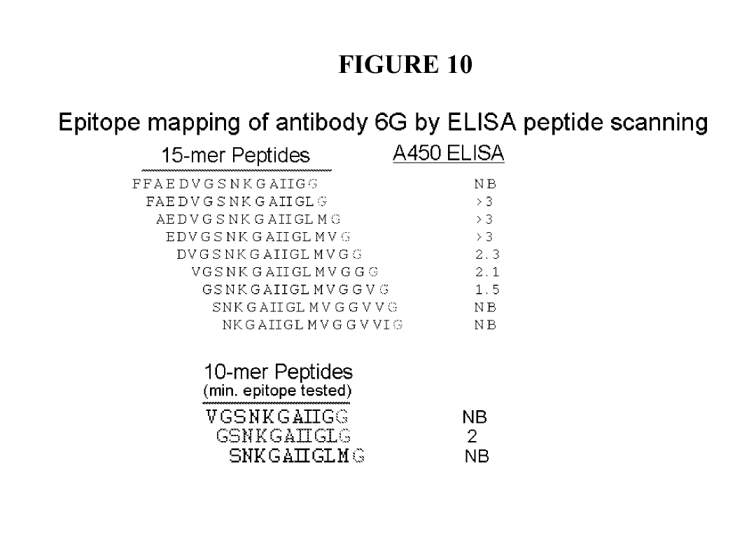

Epitope mapping of antibody 6G was performed by ELISA assay. Biotinylated 15-mer or 10-mer of various Aβ peptides (these peptides have glycine added to the C-terminal end) were immobilized on streptavidin coated plates. Antibody 6G (from 2.5 ug/ml to 10 ug/ml) was incubated with the immobilized peptides and binding was measured as described above. As shown in FIG. 10, antibody 6G binds to Aβ peptides with amino acids 20-34, 21-35, 22-36, 23-37, 24-38, 25-39 and 25-34 with a glycine at the C-terminus; but does not bind to Aβ peptides with amino acids 19-33, 26-40, 27-41, 24-33, and 26-35 having a glycine at the C-terminus of these peptides. This suggests that the epitope of antibody 6G binds includes amino acids from 25 to 34.

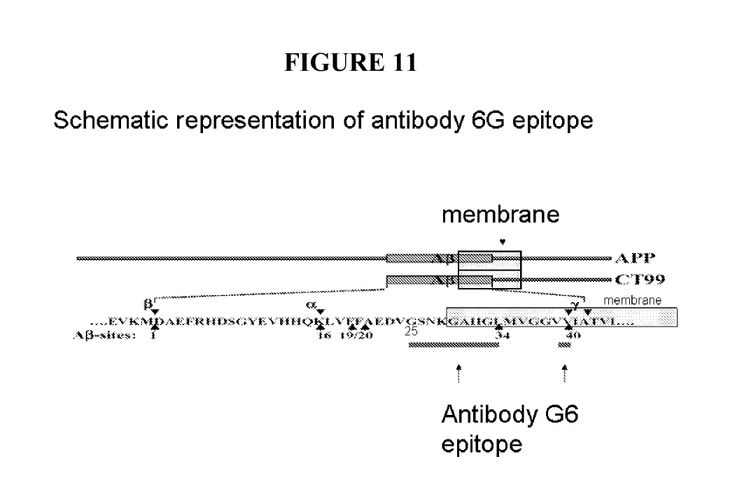

Based on data shown above, the epitope that antibody 6G binds seems to include amino acids 25-34 and 40. FIG. 11 is a schematic graph showing epitope of antibody 6G.

B. Antibody 6G does not Bind to App

To determine whether 6G binds to amyloid precursor proteins (APP), binding of 6G to cells transfected with wildtype APP was determined. HEK293 cells were transfected with a cDNA encoding wild type human amyloid precursor protein. Forty eight hours after the transfection, cells were incubated on ice for 45 minutes with monoclonal antibodies anti-Aβ₁₋₁₆, (m2324) or 6G (5 ug/ml in DMEM with 10% FCS). The cells were then washed three times in PBS for 5 minutes, fixed with 4% PFA. The cells were washed three times again in PBS, and antibody binding was detected with secondary Cy3-conjugated goat anti-mouse antibody (dilution of 1:500) from Jackson Immunoresearch under fluorescence microscope.

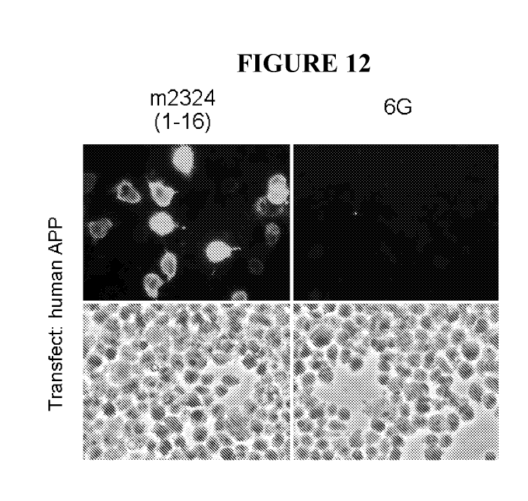

As shown in FIG. 12, anti-Aβ₁₋₁₆ antibody, which recognize N-terminal epitopes in Aβ, showed significant binding to APP precursor proteins expressed on cells. In contrast, 6G did not bind to APP expressing cells.

### Example 7

### Production and Characterization of murine antibody 7G10 (anti-Aβ₁₋₄₂/Aβ₁₋₄₃)

Mice were immunized with ˜100 μg of a peptide conjugated to KLH in adjuvant as described in Konig, G. et al, Annals New York Academy of Sciences. 777:344-55 (1996). Since positions 29-42 of the BA4 peptide lie entirely within the putative transmembrane region of APP and are hydrophobic in nature, the KLH peptide was conjugated with a hydrophilic spacer. KLH-HDGDGD-MVGGVVIA was synthesized at Anaspec, and the 5 residue spacer was sufficient to overcome the insolubility problems and extend the C-terminus away from the carrier. On the first day, mice were immunized with 100 μg 35-42/KLH peptide with CFA (complete Freud's adjuvant) subcutaneously. On day 15, mice were immunized with 100 μg peptide/KLH Ribi/alum. On day 55 mice were immunized as for Day 15. On day 95, mice were boosted 100 μg peptide/KLH intravenously.

Splenocytes were obtained from the immunized mouse, and on Day 99 were fused with P3x63Ag8.653 myeloma cells, ATCC CRL 1580, at a ratio of 10:1 with polyethylene glycol 1500. Fused cells were plated into 96-well plates in DMEM containing 20% horse serum and 2-oxaloacetate/pyruvate/insulm (Sigma) and supernatants assayed starting Day 10 after fusion using an Elisa assay and coating with 2 ug/ml Aβ1-42 (Anaspec). Positives were selected and expanded and further characterized.

Mouse sera titer on day of fusion was 1/9000 when tested for Aβ₁₋₄₂ free peptide. Affinity binding of 7G10 to Aβ1-40, 1-42, and 1-43 was analyzed by Biacore.

### Biacore Assay

Affinities of 7G10 antibody were determined using BIAcore3000® as described previously in Example 1. N-biotinylated Aβ1-40, 1-42, and 1-43 were captured on SA chip. Threefold dilution series of anti-Aβ1-40 Fabs, anti-Aβ1-42 Fabs, and anti-Aβ1-43 Fabs respectively, were injected starting with a ⅙ dilution of the 7G10 stock listed above. The chip was regenerated with an 18 sec pulse of 6% EtOH+6 mM NaOH.

| | IgG | Vol. | Fab | Vol. | |
|---|---|---|---|---|---|
| Sample | (mg/mL) | (mL) | (mg/mL) | (mL) | KD (nM) |
| Aβ₁₋₄₀ peptide | 0.672 | 2.4 | 1.458 | 0.4 | Not Applicable |
| Aβ₁₋₄₂ peptide | 0.672 | 2.4 | 1.458 | 0.4 | 37.6 |
| Aβ₁₋₄₃ peptide | 0.672 | 2.4 | 1.458 | 0.4 | 41 |

As indicated above, 7G10 had a K_{D} of 37.6 nM to Aβ₁₋₄₂ peptide and a a K_{D} of 41 nM to Aβ₁₋₄₃ peptide. No measurable binding was detected against the Aβ₁₋₄₀ peptide.

### Example 10

### Comparative Effects of Antibodies 9TL, 6G and 7G10 in Protection and Recovery of Retinal Function in Animal Model of Age-Related Macular Degeneration

### A. Experimental Protocol

Administration of antibodies. The protocol as described in Example 4 above was repeated. ApoE4-transgenic mice, aged 65 weeks or more, were assigned to one of 5 groups for eight weeks. The first group (E4-ND) was continuously maintained on a normal diet (n=6). The second group (E4-HFC-R1) was fed a high fat, cholesterol enriched (HF-C) diet 8 weeks (n=12). The third group (E4-HFC-R1) was fed an HF-C diet for 8 weeks and received weekly intraperitoneal injections of Rinat 3 (3 mg/kg) (n=12). The fourth group (E4-HFC-R2) was fed a HF-C diet for 8 weeks and received weekly intraperitoneal injections of Rinat 4 (3 mg/kg) (n=12). The fifth group (E4-HFC-R) was fed a HF-C diet for 8 weeks and received weekly intraperitoneal injections of Rinat 5 (3 mg/kg) (n=12). After completion of the study, the groups were unmasked: Rinat 3 was 7G10; Rinat 4 was deglycosylated anti-Aβ antibody 2H6; and Rinat 5 was 6G.

Fundus examination and fluorescein angiograms were performed as described previously in Example 4 above. ERG recordings were obtained after the eighth week as described previously in Example 4 above.

### Results

Recovery/Protection of Retinal Function by Administration of Deglycosylated Antibody.

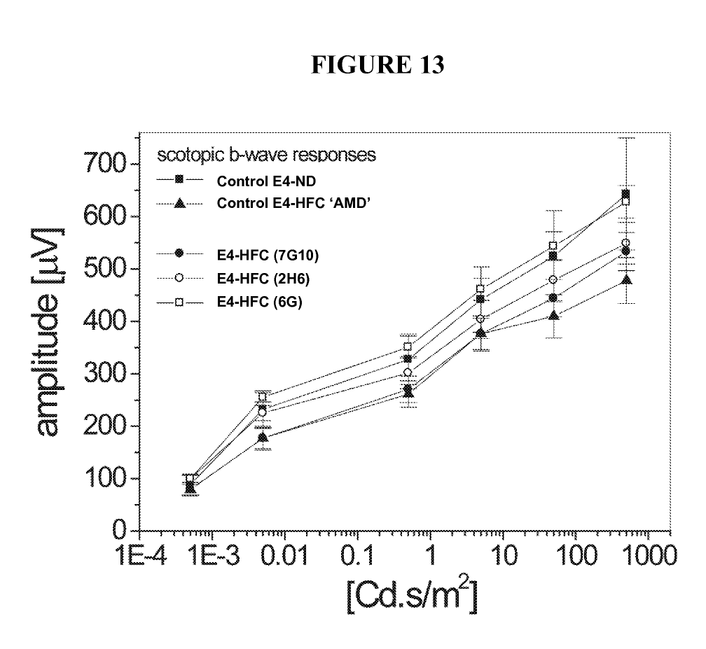

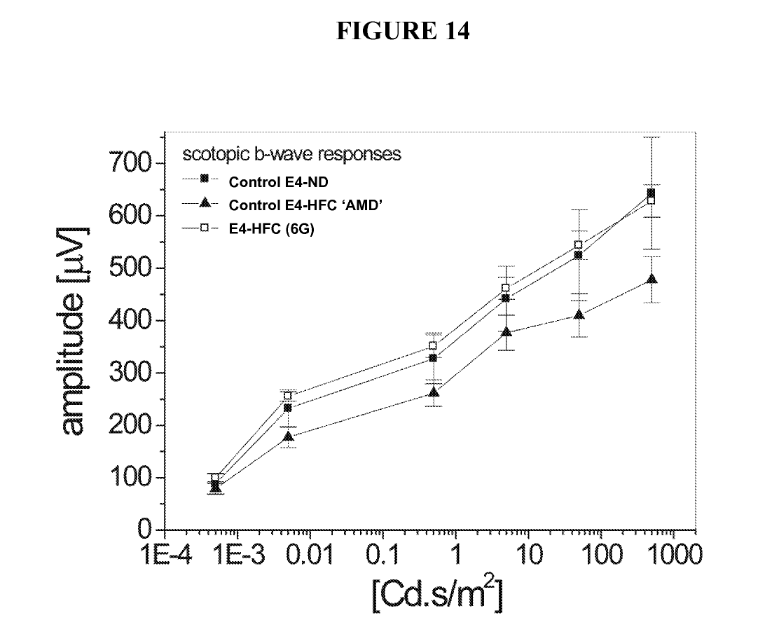

As shown in FIG. 13, the b-wave amplitudes confirm significant recovery and/or protection of retinal function in the group treated with 2H6 (E4-HFC anti-Aβ₁₋₄₀).A. The b-wave amplitudes indicate little to no recovery of the group treated with 7G10 (E4-HFC anti Aβ₁₋₄₂/Aβ₁₋₄₃). Surprisingly, the b-wave amplitudes indicate even greater recovery and/or protection of retinal function in the group treated 6G (E4-HFC anti-Aβ₁₋₄₀/Aβ₁₋₄₂). As shown in FIG. 14, the b-wave amplitude of the group treated with 6G was comparable to the control group of normal mice, indicating complete recovery and/or protection of retinal function.

### CONCLUSION

The above data demonstrate 1) the sub-RPE amyloid is pathogenic and/or toxic in AMD; 2) significant recovery/protection of retinal function as demonstrated by ERGs of mice injected with Anti-Aβ Antibody 2H6-D; and 3) complete recovery/protection of retinal function as demonstrated by ERGs of mice injected with bispecific Anti-Aβ₁₋₄₀/Aβ₁₋₄₂ 6G.

It is understood that the examples and embodiments described herein are for illustrative purposes only and that various modifications or changes in light thereof will be suggested to persons skilled in the art and are to be included within the spirit and purview of this application. All publications, patents and patent applications cited herein are hereby incorporated by reference in their entirety for all purposes to the same extent as if each individual publication, patent or patent application were specifically and individually indicated to be so incorporated by reference.

### DEPOSIT OF BIOLOGICAL MATERIAL

The following materials have been deposited with the American Type Culture Collection, 10801 University Boulevard, Manassas, Va. 20110-2209, USA (ATCC):

| | | ATCC | |
|---|---|---|---|
| Material | Antibody No. | Accession No. | Date of Deposit |
| pDb.9TL.hFc2a | 9TL heavy chain | PTA-6124 | Jul. 20, 2004 |
| pEb.9TL.hK | 9TL light chain | PTA-6125 | Jul. 20, 2004 |
| pDb.6G.hFc2a | 6G heavy chain | PTA-6786 | Jun. 15, 2005 |
| pEb.6G.hK | 6G light chain | PTA-6787 | Jun. 15, 2005 |

Vector pEb.9TL.hK is a polynucleotide encoding the 9TL light chain variable region and the light chain kappa constant region; and vector pDb.9TL.hFc2a is a polynucleotide encoding the 9TL heavy chain variable region and the heavy chain IgG2a constant region containing the following mutations: A330P331 to S330S331 (amino acid numbering with reference to the wildtype IgG2a sequence; see Eur. J. Immunol. (1999) 29:2613-2624).

Vector pEb.6G.hK is a polynucleotide encoding the 6G light chain variable region and the light chain kappa constant region; and vector pDb.6G.hFc2a is a polynucleotide encoding the 6G heavy chain variable region and the heavy chain IgG2a constant region containing the following mutations: A330P331 to S330S331 (amino acid numbering with reference to the wildtype IgG2a sequence; see Eur. J. Immunol. (1999) 29:2613-2624).

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Rinat Neuroscience Corp. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC Section 122 and the Commissioner's rules pursuant thereto (including 37 CFR Section 1.14 with particular reference to 8860G 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

Antibody sequences
9TL heavy chain variable region amino acid
sequence

(SEQ ID NO:1)

QVQLVQSGAEVKKPGASVKVSCKASGYYTEAYYIHWVRQAPGQGLEWMGR
IDPATGNTKYAPRLQDRVTMTRDTSTSTVYMELSSLRSEDTAVYYCASLY
SLPVYWGQGTTVTVSS
9TL light chain variable region amino acid
sequence

(SEQ ID NO:2)

DVVMTQSPLSLPVTLGQPASISCKSSQSLLYSDAKTYLNWFQQRPGQSPR
RLIYQISRLDPGVPDRFSGSGSGTDFTLKISRVEAEDVGVYYCLQGTHYP
VLFGQGTRLEIKRT
9TL CDR H1 (extended CDR)

(SEQ ID NO:3)

GYYTEAYYIH
9TL CDR H2 (extended CDR)

(SEQ ID NO:4)

RIDPATGNTKYAPRLQD
9TL CDR H3 (extended CDR)

(SEQ ID NO:5)

LYSLPVY
9TL CDR L1 (extended CDR)

(SEQ ID NO:6)

KSSQSLLYSDAKTYLN
9TL CDR L2 (extended CDR)

(SEQ ID NO:7)

QISRLDP
9TL CDR L3 (extended CDR)

(SEQ ID NO:8)

LQGTHYPVL
9TL heavy chain variable region nucleotide
sequence

(SEQ ID NO:9)

CAGGTGCAGCTGGTGCAGTCTGGTGCTGAGGTGAAGAAGCCTGGCGCTTC
CGTGAAGGTTTCCTGCAAAGCATCTGGTTACTATACGGAGGCTTACTATA
TCCACTGGGTGCGCCAAGCCCCTGGTCAAGGCCTGGAGTGGATGGGCAGG
ATTGATCCTGCGACTGGTAATACTAAATATGCCCCGAGGTTACAGGACCG
GGTGACCATGACTCGCGATACCTCCACCAGCACTGTCTACATGGAACTGA
GCTCTCTGCGCTCTGAGGACACTGCTGTGTATTACTGTGCCTCCCTTTAT
AGTCTCCCTGTCTACTGGGGCCAGGGTACCACTGTTACCGTGTCCTCT
9TL light chain variable region nucleotide
sequence

(SEQ ID NO:10)

GATGTTGTGATGACCCAGTCCCCACTGTCTTTGCCAGTTACCCTGGGACA
ACCAGCCTCCATATCTTGCAAGTCAAGTCAGAGCCTCTTATATAGTGATG
CCAAGACATATTTGAATTGGTTCCAACAGAGGCCTGGCCAGTCTCCACGC
CGCCTAATCTATCAGATTTCCCGGCTGGACCCTGGCGTGCCTGACAGGTT
CAGTGGCAGTGGATCAGGCACAGATTTTACACTTAAAATCAGCAGAGTGG
AGGCTGAAGATGTGGGAGTTTATTACTGCTTACAAGGTACACATTATCCG
GTGCTCTTCGGTCAAGGGACCCGCCTGGAGATCAAACGCACT
9TL heavy chain full antibody amino acid sequence
(including modified IgG2a as described herein)

(SEQ ID NO:11)

QVQLVQSGAEVKKPGASVKVSCKASGYYTEAYYIHWVRQAPGQGLEWMGR
IDPATGNTKYAPRLQDRVTMTRDTSTSTVYMELSSLRSEDTAVYYCASLY
SLPVYWGQGTTVTVSSASTKGPSVFPLAPCSRSTSESTAALGCLVKDYFP
EPVTVSWNSGALTSGVHTFPAVLQSSGLYSLSSVVTVPSSNFGTQTYTCN
VDHKPSNTKVDKTVERKCCVECPPCPAPPVAGPSVFLFPPKPKDTLMISR
TPEVTCWVDVSHEDPEVQFNWYVDGVEVHNAKTKPREEQFNSTFRVVSVL
TVVHQDWLNGKEYKCKVSNKGLPSSIEKTISKTKGQPREPQVYTLPPSRE
EMTKNQVSLTCLVKGFYPSDIAVEWESNGQPENNYKTTPPMLDSDGSFFL
YSKLTVDKSRWQQGNVFSCSVMHEALHNHYTQKSLSLSPGK
9TL light chain full antibody amino acid sequence

(SEQ ID NO:12)

DVVMTQSPLSLPVTLGQPASISCKSSQSLLYSDAKTYLNWFQQRPGQSPR
RLIYQISRLDPGVPDRFSGSGSGTDFTLKISRVEAEDVGVYYCLQGTHYP
VLFGQGTRLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
9TL heavy chain full antibody nucleotide sequence
(including modified lgG2a as described herein)

(SEQ ID NO:13)

CAGGTGCAGCTGGTGCAGTCTGGTGCTGAGGTGAAGAAGCCTGGCGCTTC
CGTGAAGGTTTCCTGCAAAGCATCTGGTTACTATACGGAGGCTTACTATA
TCCACTGGGTGCGCCAAGCCCCTGGTCAAGGCCTGGAGTGGATGGGCAGG
ATTGATCCTGCGACTGGTAATACTAAATATGCCCCGAGGTTACAGGACCG
GGTGACCATGACTCGCGATACCTCCACCAGCACTGTCTACATGGAACTGA
GCTCTCTGCGCTCTGAGGACACTGCTGTGTATTACTGTGCCTCCCTTTAT
AGTCTCCCTGTCTACTGGGGCCAGGGTACCACTGTTACCGTGTCCTCTGC
CTCCACCAAGGGCCCATCTGTCTTCCCACTGGCCCCATGCTCCCGCAGCA
CCTCCGAGAGCACAGCCGCCCTGGGCTGCCTGGTCAAGGACTACTTCCCA
GAACCTGTGACCGTGTCCTGGAACTCTGGCGCTCTGACCAGCGGCGTGCA
CACCTTCCCAGCTGTCCTGCAGTCCTCAGGTCTCTACTCCCTCAGCAGCG
TGGTGACCGTGCCATCCAGCAACTTCGGCACCCAGACCTACACCTGCAAC
GTAGATCACAAGCCAAGCAACACCAAGGTCGACAAGACCGTGGAGAGAAA
GTGTTGTGTGGAGTGTCCACCTTGTCCAGCCCCTCCAGTGGCCGGACCAT
CCGTGTTCCTGTTCCCTCCAAAGCCAAAGGACACCCTGATGATCTCCAGA
ACCCCAGAGGTGACCTGTGTGGTGGTGGACGTGTCCCACGAGGACCCAGA
GGTGCAGTTCAACTGGTATGTGGACGGAGTGGAGGTGCACAACGCCAAGA
CCAAGCCAAGAGAGGAGCAGTTCAACTCCACCTTCAGAGTGGTGAGCGTG
CTGACCGTGGTGCACCAGGACTGGCTGAACGGAAAGGAGTATAAGTGTAA
GGTGTCCAACAAGGGACTGCCATCCAGCATCGAGAAGACCATCTCCAAGA
CCAAGGGACAGCCAAGAGAGCCACAGGTGTATACCCTGCCCCCATCCAGA
GAGGAGATGACCAAGAACCAGGTGTCCCTGACCTGTCTGGTGAAGGGATT
CTATCCATCCGACATCGCCGTGGAGTGGGAGTCCAACGGACAGCCAGAGA
ACAACTATAAGACCACCCCTCCAATGCTGGACTCCGACGGATCCTTCTTC
CTGTATTCCAAGCTGACCGTGGACAAGTCCAGATGGCAGCAGGGAAACGT
GTTCTCTTGTTCCGTGATGCACGAGGCCCTGCACAACCACTATACCCAGA
AGAGCCTGTCCCTGTCTCCAGGAAAGTAATTCTAGA
9TL light chain full antibody nucleotide sequence

(SEQ ID NO:14)

GATGTTGTGATGACCCAGTCCCCACTGTCTTTGCCAGTTACCCTGGGACA
ACCAGCCTCCATATCTTGCAAGTCAAGTCAGAGCCTCTTATATAGTGATG
CCAAGACATATTTGAATTGGTTCCAACAGAGGCCTGGCCAGTCTCCACGC
CGCCTAATCTATCAGATTTCCCGGCTGGACCCTGGCGTGCCTGACAGGTT
CAGTGGCAGTGGATCAGGCACAGATTTTACACTTAAAATCAGCAGAGTGG
AGGCTGAAGATGTGGGAGTTTATTACTGCTTACAAGGTACACATTATCCG
GTGCTCTTCGGTCAAGGGACCCGCCTGGAGATCAAACGCACTGTGGCTGC
ACCATCTGTCTTCATCTTCCCTCCATCTGATGAGCAGTTGAAATCCGGAA
CTGCCTCTGTTGTGTGCCTGCTGAATAACTTCTATCCACGCGAGGCCAAA
GTACAGTGGAAGGTGGATAACGCCCTCCAATCCGGTAACTCCCAGGAGAG
TGTCACAGAGCAGGACAGCAAGGACAGCACCTACAGCCTCAGCAGCACCC
TGACCCTGAGCAAAGCAGACTACGAGAAACACAAAGTCTACGCCTGCGAA
GTCACCCATCAGGGCCTGAGTTCTCCAGTCACAAAGAGCTTCAACCGCGG
TGAGTGCTAATTCTAG
6G heavy chain variable region amino acid sequence

(SEQ ID NO:26)

QVQLVQSGAEVKKPGASVKVSCKASGYTFTTYAIHWVRQAPGQGLEWMGF
TSPYSGVSNYNQKFKGRVTMTRDTSTSTVYMELSSLRSEDTAVYYCARFD
NYDRGYVRDYWGQGTLVTVS
6G light chain variable region amino acid sequence

(SEQ ID NO:27)

DIVMTQSPDSLAVSLGERATINCRASESVDNDRISFLNWYQQKPGQPPKL
LIYAATKQGTGVPDRFSGSGSGTDFTLTISSLQAEDVAVYYCQQSKEFPW
SFGGGTKVEIKRTV
6G CDR H1 (extended CDR)

(SEQ ID NO:28)

GYTFTTYAIH
6G CDR H2 (extended CDR)

(SEQ ID NO:29)

FTSPYSGVSNYNQKFKG
6G CDR H3 (extended CDR)

(SEQ ID NO:30)

FDNYDRGYVRDY
6G CDR L1 (extended CDR)

(SEQ ID NO:31)

RASESVDNDRISFLN
6G CDR L2 (extended CDR)

(SEQ ID NO:32)

AATKQGT
6G CDR L3 (extended CDR)

(SEQ ID NO:33)

QQSKEFPWS
6G heavy chain variable region nucleotide sequence

(SEQ ID NO:34)

CAGGTGCAACTGGTGCAATCCGGTGCCGAGGTGAAAAAGCCAGGCGCCTC
CGTGAAAGTGTCCTGCAAAGCCTCCGGTTACACCTTTACCACCTATGCCA
TCCATTGGGTGCGCCAGGCCCCAGGCCAGGGTCTGGAGTGGATGGGCTTT
ACTTCCCCCTACTCCGGGGTGTCGAATTACAATCAGAAGTTCAAAGGCCG
CGTCACCATGACCCGCGACACCTCCACCTCCACAGTGTATATGGAGCTGT
CCTCTCTGCGCTCCGAAGACACCGCCGTGTATTACTGTGCCCGCTTCGAC
AATTACGATCGCGGCTATGTGCGTGACTATTGGGGCCAGGGCACCCTGGT
CACCGTCTCC
6G light chain variable region nucleotide sequence

(SEQ ID NO:35)

GACATCGTGATGACCCAGTCCCCAGACTCCCTGGCCGTGTCCCTGGGCGA
GCGCGCCACCATCAACTGCCGCGCCAGCGAATCCGTGGATAACGATCGTA
TTTCCTTTCTGAACTGGTACCAGCAGAAACCAGGCCAGCCTCCTAAGCTG
CTCATTTACGCCGCCACCAAACAGGGTACCGGCGTGCCTGACCGCTTCTC
CGGCAGCGGTTCCGGCACCGATTTCACTCTGACCATCTCCTCCCTGCAGG
CCGAAGATGTGGCAGTGTATTACTGTCAGCAGTCCAAAGAGTTTCCCTGG
TCCTTTGGCGGTGGCACCAAGGTGGAGATCAAACGCACTGTG
6G heavy chain full antibody amino acid sequence
(including modified IgG2a as described herein)

(SEQ ID NO:36)

QVQLVQSGAEVKKPGASVKVSCKASGYTFTTYAIHWVRQAPGQGLEWMGF
TSPYSGVSNYNQKFKGRVTMTRDTSTSTVYMELSSLRSEDTAVYYCARFD
NYDRGYVRDYWGQGTLVTVSSASTKGPSVFPLAPCSRSTSESTAALGCLV
KDYFPEPVTVSWNSGALTSGVHTFPAVLQSSGLYSLSSVVTVPSSNFGTQ
TYTCNVDHKPSNTKVDKTVERKCCVECPPCPAPPVAGPSVFLFPPKPKDT
LMISRTPEVTCVVVDVSHEDPEVQFNWYVDGVEVHNAKTKPREEQFNSTF
RVVSVLTVVHQDWLNGKEYKCKVSNKGLPSSIEKTISKTKGQPREPQVYT
LPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK
6G light chain full antibody amino acid sequence

(SEQ ID NO:37)

DIVMTQSPDSLAVSLGERATINCRASESVDNDRISFLNWYQQKPGQPPKL
LIYAATKQGTGVPDRFSGSGSGTDFTLTISSLQAEDVAVYYCQQSKEFPW
SFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
6G heavy chain full antibody nucleotide sequence
(including modified lgG2a as described herein)

(SEQ ID NO:38)

CAGGTGCAACTGGTGCAATCCGGTGCCGAGGTGAAAAAGCCAGGCGCCTC
CGTGAAAGTGTCCTGCAAAGCCTCCGGTTACACCTTTACCACCTATGCCA
TCCATTGGGTGCGCCAGGCCCCAGGCCAGGGTCTGGAGTGGATGGGCTTT
ACTTCCCCCTACTCCGGGGTGTCGAATTACAATCAGAAGTTCAAAGGCCG
CGTCACCATGACCCGCGACACCTCCACCTCCACAGTGTATATGGAGCTGT
CCTCTCTGCGCTCCGAAGACACCGCCGTGTATTACTGTGCCCGCTTCGAC
AATTACGATCGCGGCTATGTGCGTGACTATTGGGGCCAGGGCACCCTGGT
CACCGTCTCCTCAGCCTCCACCAAGGGCCCATCTGTCTTCCCACTGGCCC
CATGCTCCCGCAGCACCTCCGAGAGCACAGCCGCCCTGGGCTGCCTGGTC
AAGGACTACTTCCCAGAACCTGTGACCGTGTCCTGGAACTCTGGCGCTCT
GACCAGCGGCGTGCACACCTTCCCAGCTGTCCTGCAGTCCTCAGGTCTCT
ACTCCCTCAGCAGCGTGGTGACCGTGCCATCCAGCAACTTCGGCACCCAG
ACCTACACCTGCAACGTAGATCACAAGCCAAGCAACACCAAGGTCGACAA
GACCGTGGAGAGAAAGTGTTGTGTGGAGTGTCCACCTTGTCCAGCCCCTC
CAGTGGCCGGACCATCCGTGTTCCTGTTCCCTCCAAAGCCAAAGGACACC
CTGATGATCTCCAGAACCCCAGAGGTGACCTGTGTGGTGGTGGACGTGTC
CCACGAGGACCCAGAGGTGCAGTTCAACTGGTATGTGGACGGAGTGGAGG
TGCACAACGCCAAGACCAAGCCAAGAGAGGAGCAGTTCAACTCCACCTTC
AGAGTGGTGAGCGTGCTGACCGTGGTGCACCAGGACTGGCTGAACGGAAA
GGAGTATAAGTGTAAGGTGTCCAACAAGGGACTGCCATCCAGCATCGAGA
AGACCATCTCCAAGACCAAGGGACAGCCAAGAGAGCCACAGGTGTATACC
CTGCCCCCATCCAGAGAGGAGATGACCAAGAACCAGGTGTCCCTGACCTG
TCTGGTGAAGGGATTCTATCCATCCGACATCGCCGTGGAGTGGGAGTCCA
ACGGACAGCCAGAGAACAACTATAAGACCACCCCTCCAATGCTGGACTCC
GACGGATCCTTCTTCCTGTATTCCAAGCTGACCGTGGACAAGTCCAGATG
GCAGCAGGGAAACGTGTTCTCTTGTTCCGTGATGCACGAGGCCCTGCACA
ACCACTATACCCAGAAGAGCCTGTCCCTGTCTCCAGGAAAG
6G light chain full antibody nucleotide sequence

(SEQ ID NO:39)

GACATCGTGATGACCCAGTCCCCAGACTCCCTGGCCGTGTCCCTGGGCGA
GCGCGCCACCATCAACTGCCGCGCCAGCGAATCCGTGGATAACGATCGTA
TTTCCTTTCTGAACTGGTACCAGCAGAAACCAGGCCAGCCTCCTAAGCTG
CTCATTTACGCCGCCACCAAACAGGGTACCGGCGTGCCTGACCGCTTCTC
CGGCAGCGGTTCCGGCACCGATTTCACTCTGACCATCTCCTCCCTGCAGG
CCGAAGATGTGGCAGTGTATTACTGTCAGCAGTCCAAAGAGTTTCCCTGG
TCCTTTGGCGGTGGCACCAAGGTGGAGATCAAACGCACTGTGGCTGCACC
ATCTGTCTTCATCTTCCCTCCATCTGATGAGCAGTTGAAATCCGGAACTG
CCTCTGTTGTGTGCCTGCTGAATAACTTCTATCCACGCGAGGCCAAAGTA
CAGTGGAAGGTGGATAACGCCCTCCAATCCGGTAACTCCCAGGAGAGTGT
CACAGAGCAGGACAGCAAGGACAGCACCTACAGCCTCAGCAGCACCCTGA
CCCTGAGCAAAGCAGACTACGAGAAACACAAAGTCTACGCCTGCGAAGTC
ACCCATCAGGGCCTGAGTTCTCCAGTCACAAAGAGCTTCAACCGCGGTGA
GTGC
m7G10 heavy chain amino acid sequence

(SEQ ID NO:40)

EVKLVESGGDLVKPGGSLKLSCAASGFTFSTYAMSWIRQTPEKRLEWVAS
IGNSSRTYYPDSVKGRFTISRDNAGSILYLQMSSLRSEDTAIYYCARGED
GNYAWFTYWGQGTQVTVS
m7G10 light chain amino acid sequence

(SEQ ID NO:41)

DIVLTQSPATLSVTPGDSVSLSCRASQSVKNNLHWYQQKSHESPRLLIKY
TFQSMSGIPSRFSGSGSGTDFTLIINSVETEDFGMYFCQQSNRWPLTFGA
GTKLEL
m7G10 H1 CDR amino acid sequence

(SEQ ID NO:42)

TYAMS
m7G10 H2 CDR amino acid sequence

(SEQ ID NO:43)

SIGNSSRTYYPDSVKG
m7G10 H3 CDR amino acid sequence

(SEQ ID NO:44)

GEDGNYAWFTY
m7G10 L1 amino acid sequence

(SEQ ID NO:45)

RASQSVKNNLH
m7G10 L2 amino acid sequence

(SEQ ID NO:46)

YTFQSMS
m7G10 L3 amino acid sequence

(SEQ ID NO:47)

QQSNRWPLT
m7G10HC heavy chain nucleotide sequence

(SEQ ID NO:48)

GAAGTGAAGCTGGTGGAGTCTGGGGGAGACTTAGTGAAGCCTGGAGGGTC
CCTGAAACTCTCCTGTGCAGCCTCTGGATTCACTTTCAGTACCTATGCCA
TGTCTTGGATTCGCCAGACTCCAGAGAAGAGGCTGGAGTGGGTCGCCTCC
ATTGGTAATAGTAGTAGGACTTACTATCCAGACAGTGTGAAGGGCCGATT
CACCATCTCCAGAGATAATGCCGGGAGCATCCTGTACCTCCAAATGAGCA
GTCTGAGGTCTGAGGACACGGCCATTTATTATTGTGCAAGAGGGGAAGAT
GGTAACTACGCCTGGTTTACTTACTGGGGCCAAGGGACTCAGGTCACCGT
CTCC
m7G10HC light chain nucleotide sequence

(SEQ ID NO:49)

GATATTGTGCTAACTCAGTCTCCAGCCACCCTGTCTGTGACTCCAGGAGA
TAGCGTCAGTCTTTCCTGCAGGGCCAGCCAAAGTGTTAAGAACAACCTAC
ACTGGTATCAACAAAAGTCACATGAGTCTCCAAGGCTTCTCATCAAGTAT
ACTTTCCAGTCCATGTCTGGGATCCCCTCCAGGTTCAGTGGCAGTGGCTC
AGGGACAGATTTCACTCTCATTATCAACAGTGTGGAGACTGAAGATTTTG
GAATGTATTTCTGTCAACAGAGTAACCGTTGGCCGCTCACGTTCGGTGCT
GGGACCAAGCTGGAGCTG

The claimed invention is:

1. A method of treating a subject suffering from an ophthalmic disease, comprising administering to the subject an effective amount of an inhibitor of β-amyloid (Aβ) peptide.

2. The method of claim 1, wherein the antibody specifically binds to the Aβ peptide selected from the group consisting of Aβ₁₋₃₆, Aβ₁₋₃₇, Aβ₁₋₃₈, Aβ₁₋₃₉, Aβ₁₋₄₀, Aβ₁₋₄₂, and Aβ₁₋₄₃.

3. The method of claim 2, wherein the antibody specifically binds to an epitope on Aβ₁₋₄₀.

4. The method of claim 3, wherein the antibody also specifically binds to an epitope on Aβ₁₋₄₂.

5. The method of claim 1, wherein the disease is age-related macular degeneration.

6. The method of claim 2, wherein the antibody is a monoclonal antibody.

7. The method of claim 2, wherein the antibody binds to the Aβ peptide with a K_{D} of about 100 nM or less.

8. The method of claim 3, wherein the antibody binds to the Aβ₁₋₄₀ peptide with a K_{D} of about 100 nM or less.

9. The method of claim 8, wherein the antibody also binds to the Aβ₁₋₄₂ peptide with a K_{D} of about 100 nM or less.

10. The method of claim 2, wherein the antibody binds to the C-terminus of the Aβ peptide.

11. The method of claim 2, wherein the antibody binds to an epitope on Aβ₁₋₄₀ that includes amino acids 25-34 and 40.

12. The method of claim 2, wherein the antibody binds to Aβ₁₋₄₀ with higher affinity than its binding to Aβ₁₋₄₂ and Aβ₁₋₄₃, and wherein the antibody is not antibody 2294.

13. The method of claim 2, wherein the Fc region of the antibody is not N-glycosylated or has an N-glycosylation pattern that is altered with respect to a native Fc region.

14. The antibody of claim 2, wherein the antibody comprises a heavy chain variable region comprising three CDRs from antibody 6G heavy chain variable region shown in SEQ ID NO:26, and a light chain variable region comprising three CDRs from antibody 6G light variable region shown in SEQ ID NO:27.

15. The antibody of claim 2, wherein the antibody comprises a heavy chain variable region comprising the amino acid sequence shown in SEQ ID NO:26, and a light chain variable region comprising the amino acid sequence shown in SEQ ID NO:27.

16. The antibody of claim 2, wherein the antibody comprises the heavy chain amino acid sequence shown in SEQ ID NO:36, and the light chain amino acid sequence shown in SEQ ID NO:37.

17. A method of treating a subject suffering from age-related macular degeneration, comprising administering to the subject an effective amount of an wherein the antibody specifically binds to an epitope on Aβ₁₋₄₀ and Aβ₁₋₄₂.

18. The method of claim 17, wherein the administration of the antibody causes significant protection or recovery of retinal function.

19. The method of claim 17, wherein the administration of the antibody causes significant preservation or restoration of visual acuity.

20. The method of claim 17, wherein the antibody is administered by injection.
